(19) United States
(12) Patent Application Publication
(10) Pub. No.: US 2026/0092070 A1
(43) Pub. Date: Apr. 2, 2026

(54) INHIBITORS OF CANINE JANUS KINASE AND USES THEREOF

(71) Applicant: ANIMOL DISCOVERY, INC., Boston, MA (US)

(72) Inventors: Andrew Antony CALABRESE, Boston, MA (US); David T. STANTON, Boston, MA (US)

(21) Appl. No.: 19/405,620

(22) Filed: Dec. 2, 2025

Related U.S. Application Data
(63) Continuation of application No. PCT/IB2024/055314, filed on May 30, 2024.
(60) Provisional application No. 63/470,589, filed on Jun. 2, 2023.

(51) Int. Cl. C07D 487/04; A61K 31/519; A61K 31/5377; A61K 31/635; A61P 17/04; A61P 29/00

(52) U.S. Cl. CPC C07D 487/04; A61K 31/519; A61K 31/5377; A61K 31/635; A61P 17/04; A61P 29/00

(57) ABSTRACT

Provided herein are compounds having inhibitory activity toward the Janus Kinase 1 (JAK-1) enzyme, and which are useful as therapeutic and/or prophylactic agents. The compounds may be useful in treating inflammatory disorders in non-human mammals, for example, in treating canine atopic dermatitis. Also provided herein are methods for preparation of such compounds.

### CROSS-REFERENCE TO RELATED APPLICATIONS

This application is a continuation of International Application No. PCT/IB2024/055314, filed May 30, 2024, and which claims priority to U.S. Provisional Application No. 63/470,589, filed Jun. 2, 2023, each of which is herein incorporated by reference in its entirety.

### TECHNICAL FIELD

The present disclosure relates generally to canine Janus Kinase (JAK) inhibitors and methods for treating inflammatory disorders with such inhibitors.

### SEQUENCE LISTING

The instant application contains a Sequence Listing which has been submitted electronically in XML format and which is hereby incorporated by reference in its entirety. Said CRF copy, created on Dec. 2, 2025, is named A 112628_W2_SL.xml and is 4,284 bytes in size.

### BACKGROUND

Protein kinases are families of enzymes that catalyze the phosphorylation of specific amino acid residues present in certain proteins. Such protein kinases are broadly classified into tyrosine and serine/threonine kinases.

Inappropriate kinase activity, which may arise from, e.g., arising mutation, overexpression, or inappropriate regulation, as well as over- or underexpression of growth factors or cytokines, has been implicated in many diseases. These diseases include but are not limited to cancer, cardiovascular diseases, allergies, asthma and other respiratory diseases, autoimmune diseases, inflammatory diseases, bone diseases, metabolic disorders, and neurological and neurodegenerative disorders. Inappropriate kinase activity triggers a variety of biological cellular responses relating to cell growth, differentiation, survival, apoptosis, and mitogenesis, as well as cell cycle control and cell mobility, each of which have been implicated in the aforementioned diseases.

Accordingly, protein kinases have emerged as an important class of enzymes to target for therapeutic intervention. In particular, the Janus Kinases (JAKs) are a family of cellular protein tyrosine kinases that play a central role in cytokine signaling (Kisseleva et al., Gene 2002, 285, 1; Yamaoka et al., Genome Biology 2004, 5, 253). The JAKs function as dimers in the signaling process of many cytokine receptors. The JAKs comprise four family members: JAK-1, JAK-2, JAK-3, and Tyrosine kinase 2 (TYK-2). Numerous cytokines are known to activate the JAK family. Upon binding to their receptors, cytokines activate the JAK, which then phosphorylates the cytokine receptor, creating docking sites for signaling molecules. These signaling molecules include members of the signal transducer and activator of transcription (STAT) family that ultimately lead to gene expression.

The JAKs play a critical role in both innate and adaptive immunity, making them attractive targets for the treatment of inflammatory diseases. Targeting the JAK signaling pathway for autoimmune diseases is supported by the involvement of various pro-inflammatory cytokines that signal via JAK pathways in the pathogenesis of these immune-related disorders. The activation of JAK signaling initiates expression of survival factors, cytokines, chemokines, and other molecules that facilitate leukocyte cellular trafficking and cell proliferation, which contribute to inflammatory and autoimmune disorders. (O'Shea et al., N Engl J Med. 2013, 368(2), 161-70).

In view of the potential for JAK inhibitors to target pathways implicated in a variety of diseases and disorders, including inflammatory disorders, it is desirable in the art to provide further JAK inhibitors and methods of utilizing such inhibitors in the treatment of diseases and disorders responsive to such inhibitors. In particular, there is a clear unmet need for new agents to control atopic dermatitis in animals that can address certain limitations of existing therapeutic agents.

### SUMMARY

The present disclosure relates to compounds which are inhibitors of Janus Kinases (JAKs), with efficacy against canine Janus Kinase-1 (cJAK-1) and selectivity over canine Janus Kinase-2 (cJAK-2), canine Janus Kinase-3 (cJAK-3), and canine Tyrosine Kinase 2 (cTYK-2). Accordingly, the disclosed compounds can be useful as therapeutic agents for indications where immunosuppression and/or immunomodulation would be desirable, including, but not limited, to canine atopic dermatitis.

Atopic Dermatitis (AD; also known as atopic eczema) is a genetically predisposed inflammatory, pruritic, chronic or chronically relapsing skin disease. It is most commonly associated with IgE antibodies to environmental allergens. Common clinical characteristics include erythema, edema, xerosis, erosions/excoriations, oozing and crusting. The disease typically affects dogs aged 6 months to 3 years and is characterized by pruritus and secondary skin lesions of a characteristic distribution around the face (mouth, eyes), concave aspect of the ear pinnae, ventral abdomen, flexor aspects of elbow, carpal, and tarsal joints, interdigital skin, and/or perineal area. Animals with atopic dermatitis are prone to secondary skin infections, ear infections and yeast infections. Atopic dermatitis cannot be cured. Therefore, the goal is to manage the disease to improve quality of life of canines and their owners.

The market for treating atopic dermatitis in animals has historically been dominated by corticosteroids and antihistamines, each suffering from various liabilities. For example, corticosteroids cause undesirable side effects in animals, specifically in companion animals such as dogs, while antihistamines suffer from poor efficacy. Particularly, the short- and long-term side effects of corticosteroids include polydipsia, polyphagia, polyuria, pancreatitis, gastrointestinal ulceration, lipidemias, diabetes, muscle wasting and iatrogenic Cushing's syndrome. Further, the complicated dosing schedules can be challenging to dogs and their owners. A canine formulation of cyclosporine (ATOPIC A™) is marketed for atopic dermatitis, but is expensive, has a slow onset of efficacy, and exhibits gastrointestinal tolerability issues. In 2013, the United States FDA approved APOQUEL® (oclacitinib; N-methyl[trans-4-(methyl-7H-pyrrolo[2,3-d]pyrimidin-4-ylamino) cyclohexyl]methanesulfonamide (2Z)-2-butenedioate; Zoetis) for the treatment of pruritus associated with allergic dermatitis and control of atopic dermatitis in dogs at least 12 months of age. APOQUEL® is a relatively selective JAK-1 and JAK-3 inhibitor, blocking the effects of inflammatory cytokines released from activated lymphocytes (IL-2, -4, -6, -13) as well as IL-31, a cytokine directly involved in the sensation of itch. Despite its commercial success, APOQUEL® requires twice daily oral dosing during the initial induction phase, which lasts up to two weeks. In view of the challenges associated with orally medicating companion animals, it would be desirable to provide an alternative therapeutic agent with properties allowing once daily dosing.

Compounds of the present disclosure are JAK inhibitors with potency, efficacy, and selectivity for human JAK-1. In view of the very high homology between canine and human JAKs, such efficacy and selectivity obtained with respect to human subtypes is an appropriate surrogate for the canine subtypes, and it is expected that the canine in vitro pharmacological profile will mirror the human in vitro profile. The activity profile and selectivity for JAK-1 may allow for once-daily dosing while providing desirable efficacy and tolerability. These compounds therefore represent a valuable alternative to currently available therapeutic agents for the treatment of chronic pruritus and inflammation associated with atopic dermatitis. Accordingly, the present disclosure provides compounds of Formula I, their use as cJAK-1 inhibitors for the treatment of canine atopic dermatitis, pharmaceutical compositions containing these compounds, and methods for the preparation of these compounds.

In one aspect is provided a compound having a structure according to Formula (I):

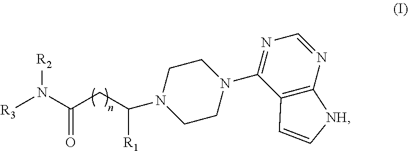

or a pharmaceutically acceptable salt or solvate thereof, wherein:
R₁ is H or optionally substituted C₁-C₃ alkyl;
n=0 or 1;
R₂ is H or CH₃;
R₃ is C₁-C₄ alkyl, phenyl,

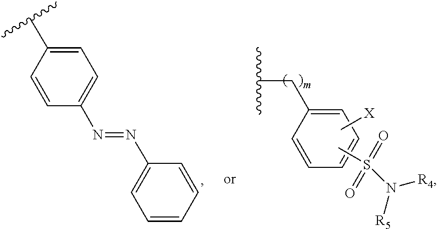

wherein:
   m is 0 or 1;
   X is H, F, Cl, OCH₃, or CH₃,
   R₄ is H or CH₃,
   R₅ is H, C₁-C₄ alkyl, C₃-C₅ cycloalkyl, CH₂CH₂OCH₃, or 2-pyrimidinyl; or
   R₄ and R₅, together with the included N atom, form a 4-, 5- or 6-membered ring,
wherein said 6-membered ring optionally includes one additional heteroatom selected from N and O; or
R₂ and R₃, together with the included N atom, form a 6-membered ring, optionally substituted with C₁-C₄ alkyl, C₇ to C₁₂ aralkyl, or —C(O)—NR₆R₇,
   wherein:
R₆ is H or C₁-C₄ alkyl;
R₇ is H, C₁-C₄ alkyl, phenyl; or
R₆ and R₇, together with the included N atom, form a 6-membered ring which may optionally be substituted with C₁-C₄alkyl, and wherein said 6-membered ring optionally includes one additional N atom.

In some embodiments, R₂ is H and R₃ is:

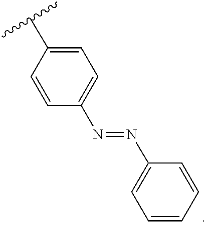 [SMILES: *c1ccc(/N=N/c2ccccc2)cc1]

In some embodiments, n is 1 and R₁ is H.

In some embodiments, n is 1 and R₁ is CH₃.

In some embodiments, R₃ is:

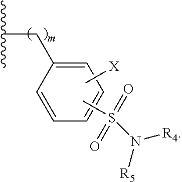

In some embodiments, m is 0.

In some embodiments, R₄ and R₅ are each H.

In some embodiments, the compound of Formula I is selected from the group consisting of:

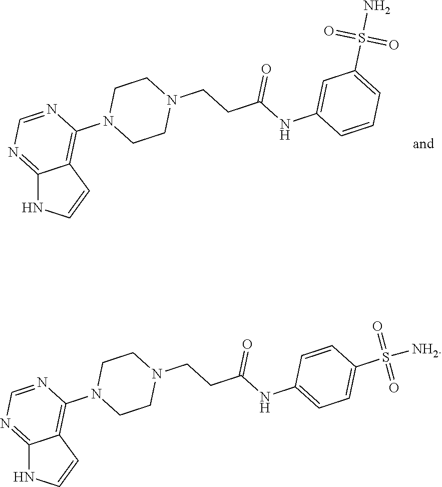 [SMILES: NS(=O)(=O)c1cccc(NC(=O)CCN2CCN(c3ncnc4[nH]ccc34)CC2)c1 ; NS(=O)(=O)c1ccc(NC(=O)CCN2CCN(c3ncnc4[nH]ccc34)CC2)cc1]

In some embodiments, m is 1.

In some embodiments, R₄ and R₅ are each CH₃.

In some embodiments, the compound of Formula I is:

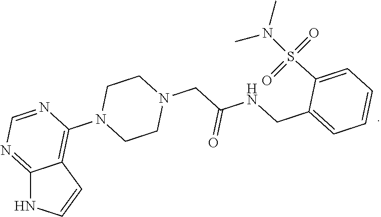 [SMILES: CN(C)S(=O)(=O)c1ccccc1CNC(=O)CN1CCN(c2ncnc3[nH]ccc23)CC1]

In some embodiments:
R₁ is H;
R₂ is H;
R₄ is H; and
R₅ is selected from the group consisting of C₁-C₄ alkyl, C₃-C₅ cycloalkyl, CH₂CH₂OCH₃, and 2-pyrimidinyl.

In some embodiments:
R₁ is CH₃;
R₂ is H;
R₄ is H; and
R₅ is selected from the group consisting of C₁-C₄ alkyl, C₃-C₅ cycloalkyl, CH₂CH₂OCH₃, and 2-pyrimidinyl.

In some embodiments, the compound of Formula I is selected from the group consisting of

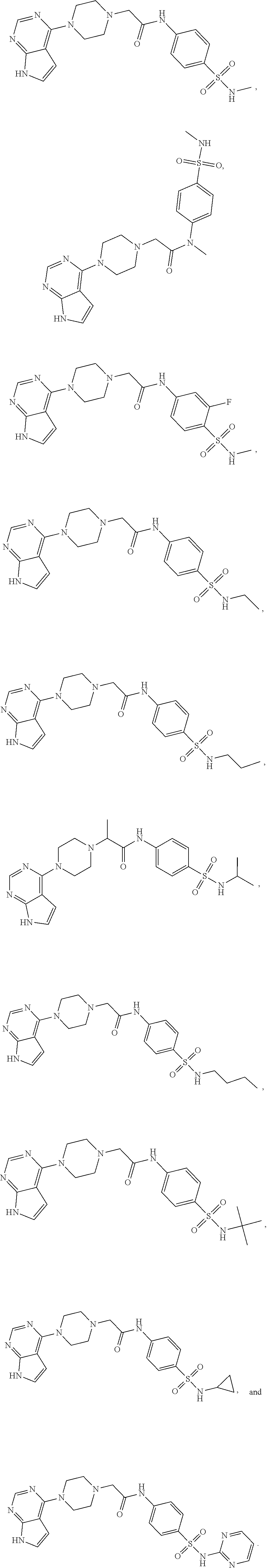

In some embodiments.
R₁ is H or CH₃;
R₂ is H; and
R₄ and R₅ are each independently selected from the group consisting of C₁-C₄ alkyl, C₃-C₅ cycloalkyl, CH₂CH₂OCH₃, and 2-pyrimidinyl.

In some embodiments, the compound of Formula I is selected from the group consisting of

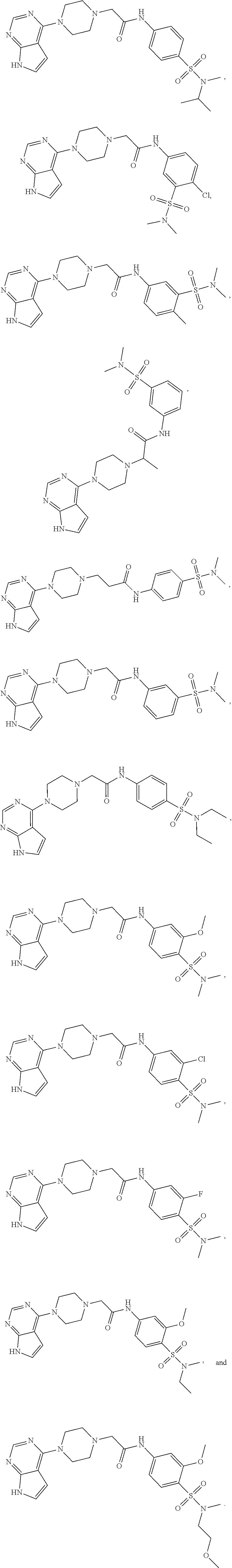

In some embodiments:
R₁ is H or CH₃;
R₂ is H; and
R₄ and R₅ together with the included N atom, form a 4-, 5- or 6-membered ring, wherein said 6-membered ring optionally includes one additional heteroatom selected from N and 0;

In some embodiments, the compound of Formula I is selected from the group consisting of

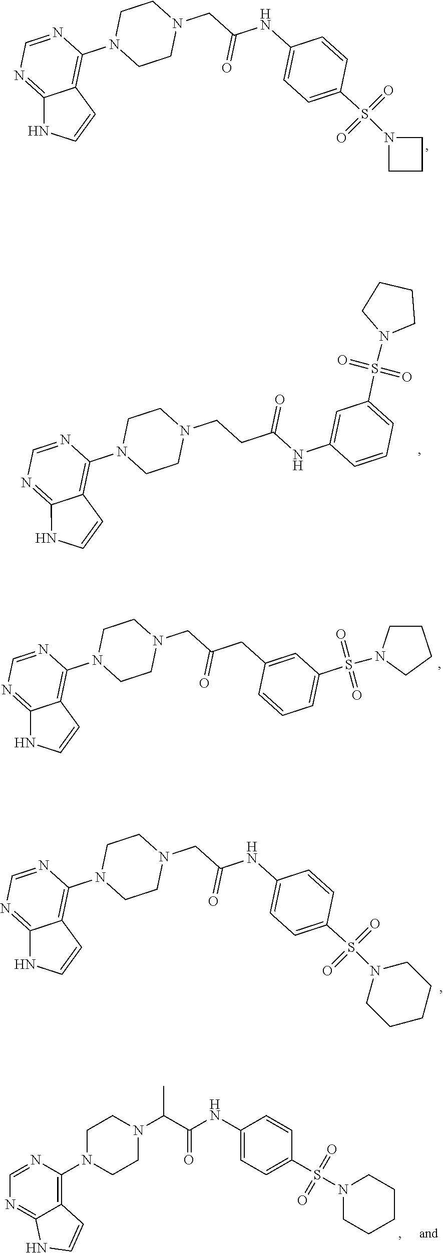 [SMILES: O=C(CN1CCN(c2ncnc3[nH]ccc23)CC1)Nc1ccc(S(=O)(=O)N2CCC2)cc1 ; O=C(CCN1CCN(c2ncnc3[nH]ccc23)CC1)Nc1cccc(S(=O)(=O)N2CCCC2)c1 ; O=C(Cc1cccc(S(=O)(=O)N2CCCC2)c1)CN1CCN(c2ncnc3[nH]ccc23)CC1 ; O=C(CN1CCN(c2ncnc3[nH]ccc23)CC1)Nc1ccc(S(=O)(=O)N2CCCCC2)cc1 ; CC(C(=O)Nc1ccc(S(=O)(=O)N2CCCCC2)cc1)N1CCN(c2ncnc3[nH]ccc23)CC1]

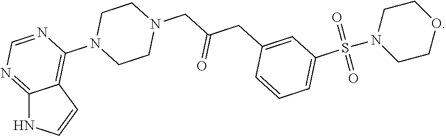 [SMILES: O=C(Cc1cccc(S(=O)(=O)N2CCOCC2)c1)CN1CCN(c2ncnc3[nH]ccc23)CC1]

In some embodiments, the compound of Formula I is

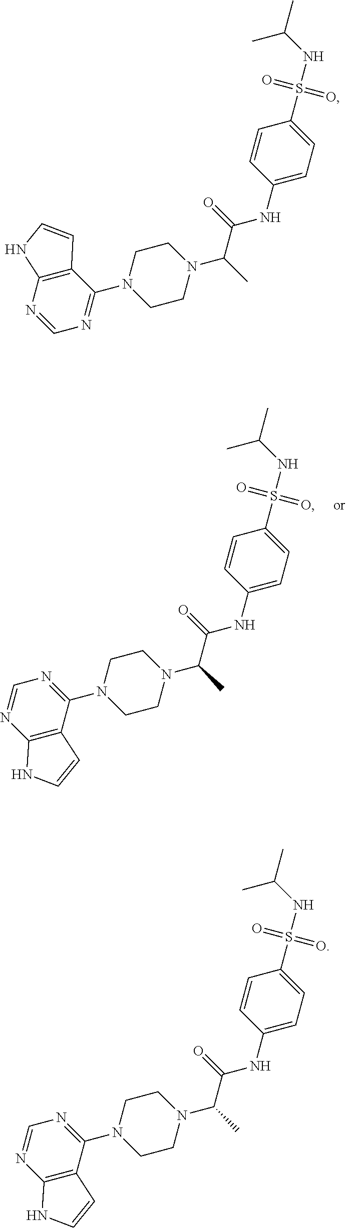 [SMILES: CC(C)NS(=O)(=O)c1ccc(NC(=O)C(C)N2CCN(c3ncnc4[nH]ccc34)CC2)cc1 ; CC(C)NS(=O)(=O)c1ccc(NC(=O)[C@@H](C)N2CCN(c3ncnc4[nH]ccc34)CC2)cc1 ; CC(C)NS(=O)(=O)c1ccc(NC(=O)[C@H](C)N2CCN(c3ncnc4[nH]ccc34)CC2)cc1]

In some embodiments, the compound of Formula I is

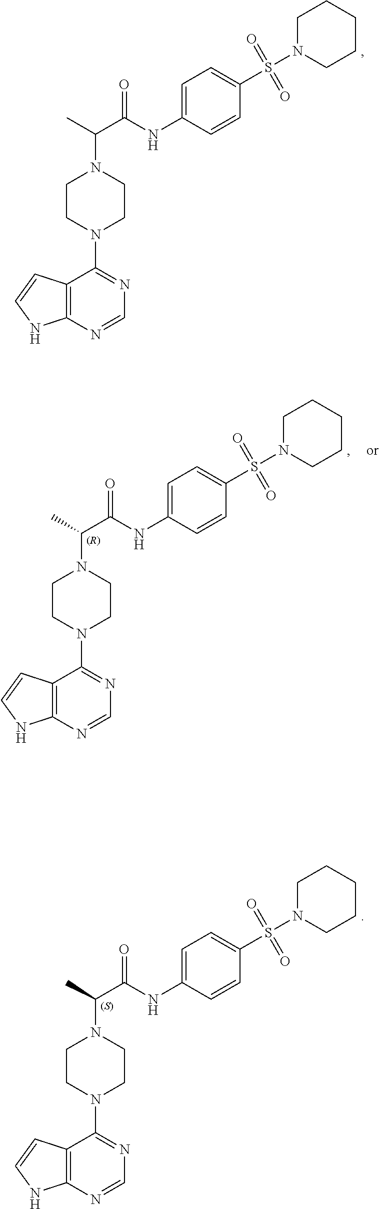

In some embodiments, R₂ and R₃, together with the included N atom, form a 6-membered ring, optionally substituted with C₁-C₄ alkyl, C₇ to C₁₂ aralkyl, or —C(O)—NR₆R₇.

In some embodiments, the compound of Formula I has a structure according to Formula IL:

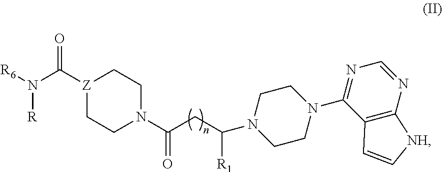

wherein Z is C or N.

In some embodiments, the compound of Formula II has a structure selected from the group consisting of

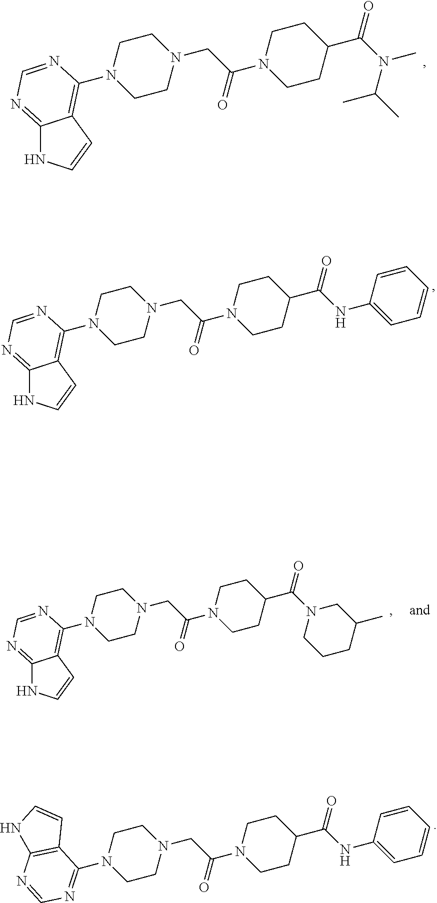 [SMILES: CC(C)N(C)C(=O)C1CCN(C(=O)CN2CCN(c3ncnc4[nH]ccc34)CC2)CC1 ; O=C(Nc1ccccc1)C1CCN(C(=O)CN2CCN(c3ncnc4[nH]ccc34)CC2)CC1 ; CC1CCCN(C(=O)C2CCN(C(=O)CN3CCN(c4ncnc5[nH]ccc45)CC3)CC2)C1 ; O=C(Nc1ccccc1)C1CCN(C(=O)CN2CCN(c3ncnc4[nH]ccc34)CC2)CC1]

In some embodiments, the compound of Formula I has a structure:

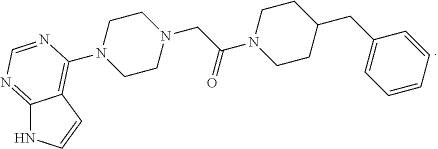 [SMILES: O=C(CN1CCN(c2ncnc3[nH]ccc23)CC1)N1CCC(Cc2ccccc2)CC1]

In another aspect is provided a pharmaceutical composition comprising a therapeutically effective amount of a compound of Formula I, or a pharmaceutically acceptable salt or solvate thereof.

In still another aspect is provided a method for treating allergic reactions, allergic dermatitis, atopic dermatitis, eczema, or pruritus in a mammal comprising administering to a mammal in need thereof a therapeutically effective amount of a compound of Formula I, or a pharmaceutically acceptable salt or solvate thereof, or a composition of any thereof.

In some embodiments, the mammal is a companion animal. In some embodiments, the companion animal is a dog.

In some embodiments, the compound of Formula I is administered orally, parenterally, or topically. In some embodiments, the compound of Formula I is administered orally once daily.

These and other features, aspects, and advantages of the present disclosure will be apparent from a reading of the following detailed description. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise. It will therefore be appreciated that this Summary is provided merely for purposes of summarizing some example implementations so as to provide a basic understanding of some aspects of the disclosure. Accordingly, it will be appreciated that the above-described example implementations are merely examples and should not be construed to narrow the scope or spirit of the disclosure in any way. Other example implementations, aspects, and advantages will become apparent from the following detailed description.

### DETAILED DESCRIPTION

The present disclosure will now be described more fully hereinafter with reference to example embodiments thereof. Before describing several example embodiments of the technology, it is to be understood that the technology is not limited to the details of construction or process steps set forth in the following description. The technology is capable of other embodiments and of being practiced or being carried out in various ways.

The following description sets forth numerous exemplary configurations, methods, parameters, and the like in order to provide a thorough understanding of various embodiments of the disclosure. It should be recognized, however, that such description is not intended as a limitation on the scope of the present disclosure but is instead provided as a description of exemplary embodiments.

The present disclosure is generally directed to compounds of Formula I.

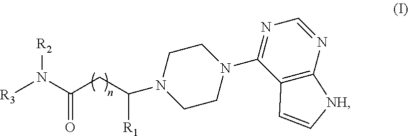

including stereoisomers, tautomers, solvates, and pharmaceutically acceptable salts thereof, and pharmaceutical compositions comprising said compounds, or stereoisomers, tautomers, solvates, and pharmaceutically acceptable salts thereof. The disclosure particularly relates to such compounds, stereoisomers, tautomers, solvates, and pharmaceutically acceptable salts thereof with canine Janus Kinase-1 (cJAK-1) inhibitory activity. Such compounds and pharmaceutical compositions of the disclosure may be useful for treating canine atopic dermatitis and the chronic pruritus and inflammation associated therewith. The compounds, compositions, and methods of treatment are further described herein below.

### Definitions

Unless defined otherwise, all technical and scientific terms used herein have the same meaning as is commonly understood by one of skill in the art to which this disclosure belongs. With respect to the terms used in this disclosure, the following definitions are provided. This application will use the following terms as defined below unless the context of the text in which the term appears requires a different meaning.

The articles “a” and “an” as used in this disclosure may refer to one or more than one (i.e., to at least one) of the grammatical object of the article. By way of example, “an element” may mean one element or more than one element.

The term “and/or” as used in this disclosure may mean either “and” or “or” unless indicated otherwise.

The term “about” used throughout this specification is used to describe and account for small fluctuations. For example, the term “about” can refer to less than or equal to +10%, less than or equal to ±5%, less than or equal to ±2%, less than or equal to +1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value. For instance, “about 5.0” must include 5.0.

Unless the context requires otherwise, throughout the present specification and claims, as used herein, the terms “including,” “containing,” and “comprising” are used in their open, non-limiting sense.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term—C_{x-y}alkylene—refers to a substituted or unsubstituted alkylene chain with from x to y carbons in the alkylene chain. For example—C₁₋₆alkylene—may be selected from methylene, ethylene, propylene, butylene, pentylene, and hexylene, any one of which is optionally substituted.

“Alkoxy” refers to a radical bonded through an oxygen atom of the formula—o-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term—C_{x-y}alkenylene—refers to a substituted or unsubstituted alkenylene chain with from x to y carbons in the alkenylene chain. For example, —C₂₋₆alkenylene—may be selected from ethenylene, propenylene, butenylene, pentenylene, and hexenylene, any one of which is optionally substituted.

An alkenylene chain may have one double bond or more than one double bond in the alkenylene chain. The term—C_{x-y}alkynylene—refers to a substituted or unsubstituted alkynylene chain with from x to y carbons in the alkenylene chain. For example, —C₂₋₆alkenylene—may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atoms (i.e., C₂ alkenylene). In other embodiments, an alkenylene comprises five to eight carbon atoms (i.e., C₅-C₈alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atoms (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R^{e} is an alkynylene chain as defined above.

“Carbocycle” refers to a saturated, unsaturated or aromatic ring in which each atom of the ring is carbon.

Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., C₁, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Heterocycle” refers to a saturated, unsaturated or aromatic ring comprising one or more heteroatoms.

Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. In some embodiments, the heterocycle is a heteroaryl. In some embodiments, the heterocycle is a heterocycloalkyl. “Heterocyclene” refers to a divalent heterocycle linking the rest of the molecule to a radical group

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heterocycloalkylalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkylene chain at the nitrogen atom.

“Heteroaryl” or “aromatic heterocycle” refers to a radical derived from a 3- to 12-membered aromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hickel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkylene chain at the nitrogen atom.

The term “amino” as used herein refers to —NH₂.

The terms “hydroxy” and “hydroxyl” refer to —OH.

The term “oxo” as used herein refers to an “═O” group. It can also be abbreviated herein as C(O) or as C═O.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with pennitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino(═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

As used herein, the term “unsubstituted” means that the specified group bears no substituents beyond the moiety recited (e.g., where valency is satisfied by hydrogen).

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

In certain embodiments, the compounds of the disclosure may contain asymmetric or chiral centers, and, therefore, exist in different stereoisomeric forms. The term “stereoisomers” may refer to the set of compounds which have the same number and type of atoms and share the same bond connectivity between those atoms but differ in three-dimensional structure. The term “stereoisomer” may refer to any member of this set of compounds. For instance, a stereoisomer may be an enantiomer or a diastereomer. It is intended that all stereoisomeric forms of the compounds of the disclosure as well as mixtures thereof, including racemic mixtures, form part of the present disclosure.

When stereochemistry is not specified, certain molecules described herein include isomers, such as enantiomers and diastereomers, mixtures of enantiomers, including racemates, mixtures of diastereomers, and other mixtures thereof, to the extent they can be made by one of ordinary skill in the art by routine experimentation. In certain embodiments, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration.

In certain embodiments, the chiral centers of the present disclosure may have the S or R configuration as defined by the IUPAC 1974 Recommendations.

The term “radical of a compound” as used herein refers to a structure derived from a parent compound by removal of one or more atoms, e.g., hydrogen atoms. In one embodiment, a “radical of a compound” is a monovalent radical derived from the removal of one hydrogen atom from the parent compound.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” and the like.

Wherever a substituent is depicted as a di-radical (i.e., has two points of attachment to the rest of the molecule), it is to be understood that the substituent can be attached in any directional configuration unless otherwise indicated.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

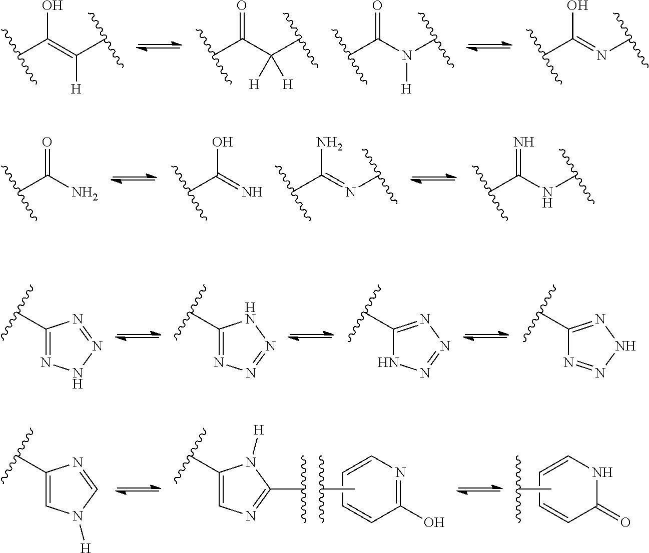

“Stable compound” and “stable structure” may indicate a compound that is sufficiently robust to survive isolation to a useful degree of purity from a reaction mixture, and formulation into an efficacious therapeutic agent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc: (8) excipients, such as cocoa butter and suppository waxes: (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “carrier,” as used in this disclosure, may encompass carriers, excipients, and diluents and may mean a material, composition or vehicle, such as a liquid or solid filler, diluent, excipient, solvent or encapsulating material, involved in carrying or transporting a pharmaceutical agent, such as one or more compounds, or pharmaceutically acceptable salts, solvates (e.g., hydrates), isomers (e.g., stereoisomers), and tautomers thereof, of the disclosure, from one organ, or portion of the body, to another organ, or portion of the body of a subject.

Carriers should be selected on the basis of compatibility and the release profile properties of the desired dosage form. Exemplary carrier materials may include, e.g., adjuvants, binders, suspending agents, disintegration agents, filling agents, surfactants, solubilizers, stabilizers, lubricants, wetting agents, diluents, spray-dried dispersions, and the like. See, e.g., Hoover, John E., Remington's Pharmaceutical Sciences, Mack Publishing Co., Easton, Pa. 1975. Exemplary carrier materials may also include without limitation any adjuvant, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The terms “pharmaceutically acceptable” or “pharmacologically acceptable” may refer to a material which is not biologically, or otherwise, undesirable—the material may be administered to an individual without causing any substantially undesirable biological effects or interacting in a deleterious manner with any of the components of the composition in which it is contained.

A “pharmaceutical composition” may refer to a formulation of a compound of the disclosure and a medium generally accepted in the art for the delivery of the biologically active compound to a subject, e.g., mammals or humans. Such a medium may include all pharmaceutically acceptable carriers therefor.

The terms “subject,” “individual,” and “patient” may be used interchangeably and refer to non-human mammals (e.g., non-human primates, canines, equines, felines, porcines, bovines, ungulates, lagomorphs, and the like).

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used.

The term “effective amount” or “therapeutically effective amount” refers to that amount of a compound or salt described herein that is sufficient to affect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. In some embodiments, a therapeutic benefit may include alleviating, abating or ameliorating symptoms of a disease or condition, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In some embodiments, the therapeutic benefit includes the eradication or amelioration of the underlying disorder being treated. In some embodiments, the therapeutic benefit includes the eradication or amelioration of one or more of the physiological symptoms associated with the underlying disorder, such as observing an improvement in the subject, notwithstanding that the subject may still be afflicted with the underlying disorder.

In certain embodiments, for prophylactic benefit, the compounds or compositions are administered to a subject at risk of developing a particular disease, or to a subject exhibiting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

In certain embodiments, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

### Compounds of the Disclosure

Generally, the present disclosure provides compounds having a structure according to Formula I:

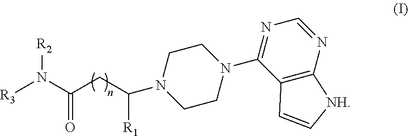

In some embodiments, such compounds exhibit affinity, inhibitory activity, or both toward the canine JAK1 subtype, and may be useful for treating diseases and disorders mediated by the canine JAK1 subtype, such as canine atopic dermatitis. In some embodiments, a compound according to Formula I exhibits selectivity for the canine JAK1 subtype relative to other canine JAK subtypes, such as cJAK2, cJAK3, canine Tyrosine Kinase 2 (cTYK2), or combinations thereof. As described herein, such activity and selectivity may be desirable in providing a companion animal therapeutic agent with improved tolerability relative to known companion animal therapeutic agents for the treatment of e.g., canine atopic dermatitis, and may provide the potential for once daily dosing.

Accordingly, in one aspect is provide a compound having a structure according to Formula I,

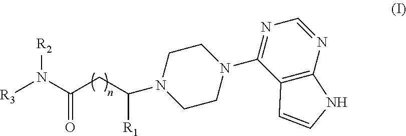

or a pharmaceutically acceptable salt or solvate thereof, wherein:
R₁ is H or optionally substituted C₁-C₃ alkyl;
n=0 or 1;
R₂ is H or CH₃;
R₃ is C₁-C₄ alkyl, phenyl,

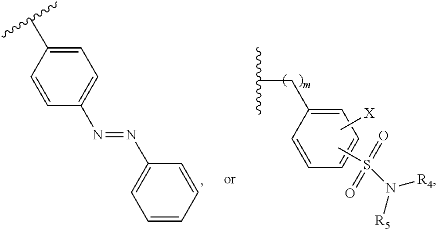

wherein:
   m is 0 or 1;
   X is H, F, Cl, OCH₃, or CH₃,
   R₄ is H or CH₃,
   R₅ is H, C₁-C₄ alkyl, C₃-C₅ cycloalkyl, CH₂CH₂OCH₃, or 2-pyrimidinyl; or
   R₄ and R₅, together with the included N atom, form a 4-, 5- or 6-membered ring,
wherein said 6-membered ring optionally includes one additional heteroatom selected from N and O; or
R₂ and R₃, together with the included N atom, form a 6-membered ring, optionally substituted with C₁-C₄ alkyl, C₇ to C₁₂ aralkyl, or —C(O)—NR₆R₇,
   wherein:
R₆ is H or C₁-C₄ alkyl;
R₇ is H, C₁-C₄ alkyl, phenyl; or
R₆ and R₇, together with the included N atom, form a 6-membered ring which may optionally be substituted with C₁-C₄alkyl, and wherein said 6-membered ring optionally includes one additional N atom.

In some embodiments R₁ is H.

In some embodiments R₁ is CH₃.

In some embodiments, R₂ is H.

In some embodiments, R₂ is H and R₃ is:

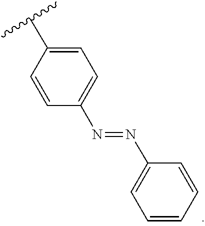 [SMILES: *c1ccc(/N=N/c2ccccc2)cc1]

In some embodiments, n is 1 and R₁ is H.

In some embodiments, R₃ is:

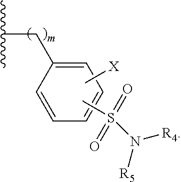

In some embodiments, m is 0. In some embodiments, m is 1.

In some embodiments, X is H. In some embodiments, X is F or Cl. In some embodiments, X is OCH₃. In some embodiments, X is CH₃.

In some embodiments, R₄ is H or CH₃. In some embodiments, R₄ is H. In some embodiments, R₄ is CH₃.

In some embodiments, R₅ is H, C₁-C₄ alkyl, C₃-C₅ cycloalkyl, CH₂CH₂OCH₃, or 2-pyrimidinyl.

In some embodiments, R₄ and R₅, together with the included N atom, form a 4-, 5- or 6-membered ring, wherein said 6-membered ring optionally includes one additional heteroatom selected from N and O.

In some embodiments, m is 0 and R₄ and R₅ are each H.

In some embodiments, the compound of Formula I is selected from the group consisting of

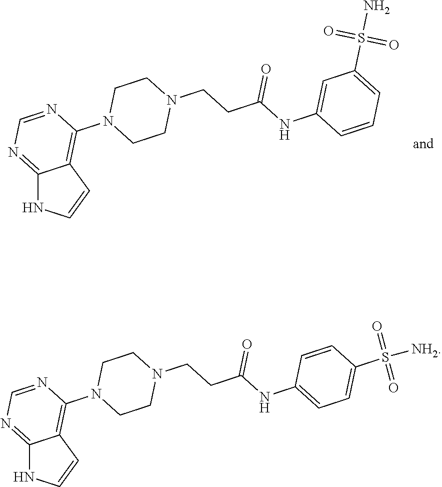 [SMILES: NS(=O)(=O)c1cccc(NC(=O)CCN2CCN(c3ncnc4[nH]ccc34)CC2)c1 ; NS(=O)(=O)c1ccc(NC(=O)CCN2CCN(c3ncnc4[nH]ccc34)CC2)cc1]

In some embodiments, m is 1.

In some embodiments, R₄ and R₅ are each CH₃.

In some embodiments, the compound of Formula I is:

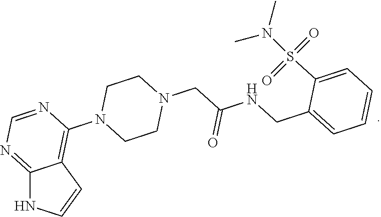 [SMILES: CN(C)S(=O)(=O)c1ccccc1CNC(=O)CN1CCN(c2ncnc3[nH]ccc23)CC1]

In some embodiments, R₁, R₂ and R₄ are H; and R₅ is selected from the group consisting of C₁-C₄ alkyl, C₃-C₅ cycloalkyl, CH₂CH₂OCH₃, and 2-pyrimidinyl.

In some embodiments, R₁ is CH₃; R₂ and R₄ are H; and R₅ is selected from the group consisting of C₁-C₄ alkyl, C₃-C₅ cycloalkyl, CH₂CH₂OCH₃, and 2-pyrimidinyl.

In some embodiments, R₁ is H or CH₃; R₂ and R₄ are H; and it is C₁-C₄ alkyl. In some embodiments, R₁ is H or CH₃; R₂ and R₄ are H; and it is C₃-C₅ cycloalkyl. In some embodiments, R₁ is H or CH₃; R₂ and R₄ are H; and R₅ is CH₂CH₂OCH₃. In some embodiments, R₁ is H or CH₃; R₂ and R₄ are H; and it is 2-pyrimidinyl.

In some embodiments, the compound of Formula I s selected from the group consisting of

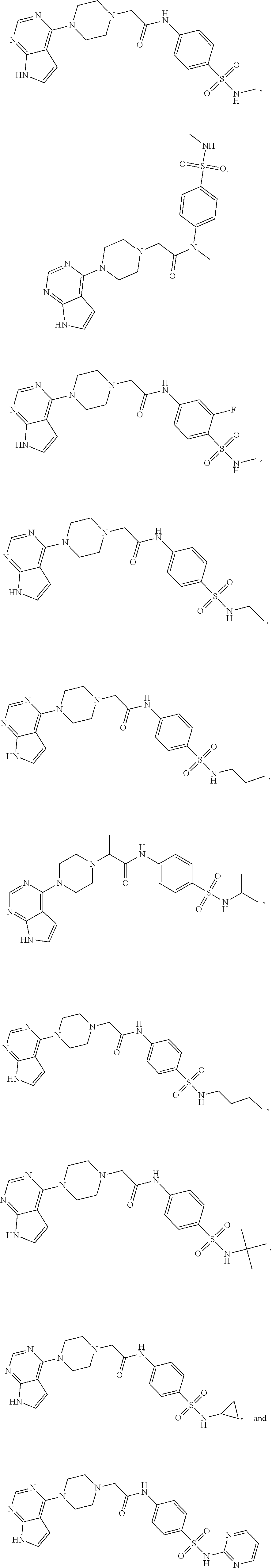

In some embodiments, the compound of Formula I s selected from the group consisting of

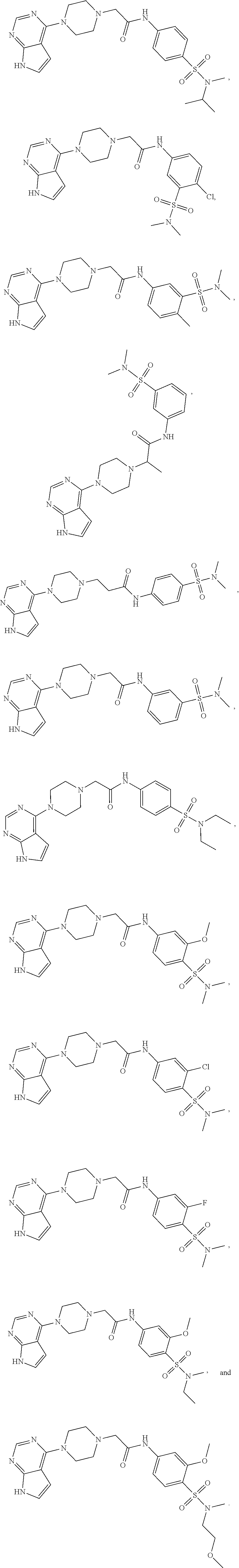

In some embodiments, the compound of Formula I is selected from the group consisting of

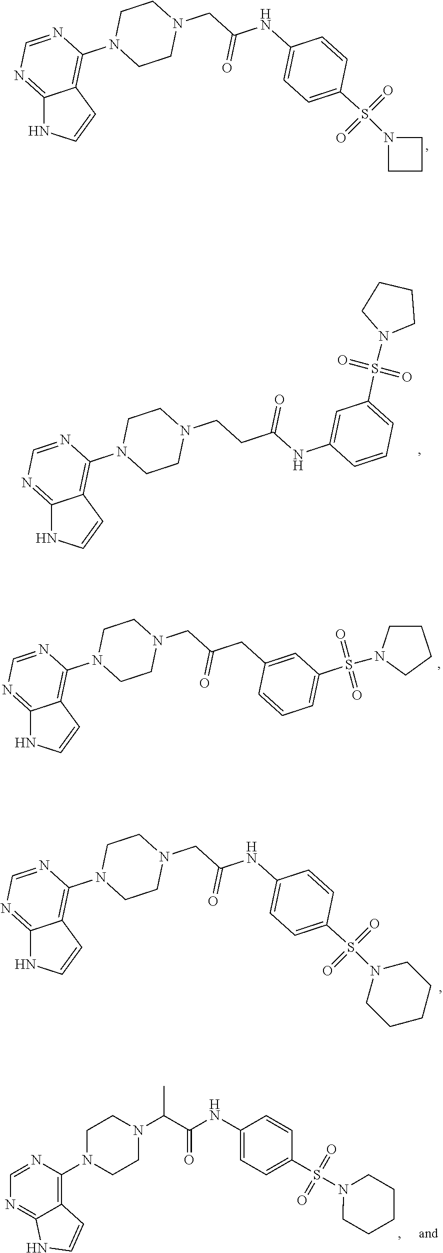 [SMILES: O=C(CN1CCN(c2ncnc3[nH]ccc23)CC1)Nc1ccc(S(=O)(=O)N2CCC2)cc1 ; O=C(CCN1CCN(c2ncnc3[nH]ccc23)CC1)Nc1cccc(S(=O)(=O)N2CCCC2)c1 ; O=C(Cc1cccc(S(=O)(=O)N2CCCC2)c1)CN1CCN(c2ncnc3[nH]ccc23)CC1 ; O=C(CN1CCN(c2ncnc3[nH]ccc23)CC1)Nc1ccc(S(=O)(=O)N2CCCCC2)cc1 ; CC(C(=O)Nc1ccc(S(=O)(=O)N2CCCCC2)cc1)N1CCN(c2ncnc3[nH]ccc23)CC1]

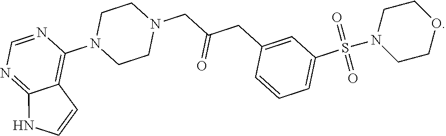 [SMILES: O=C(Cc1cccc(S(=O)(=O)N2CCOCC2)c1)CN1CCN(c2ncnc3[nH]ccc23)CC1]

In some embodiments, the compound of Formula I is

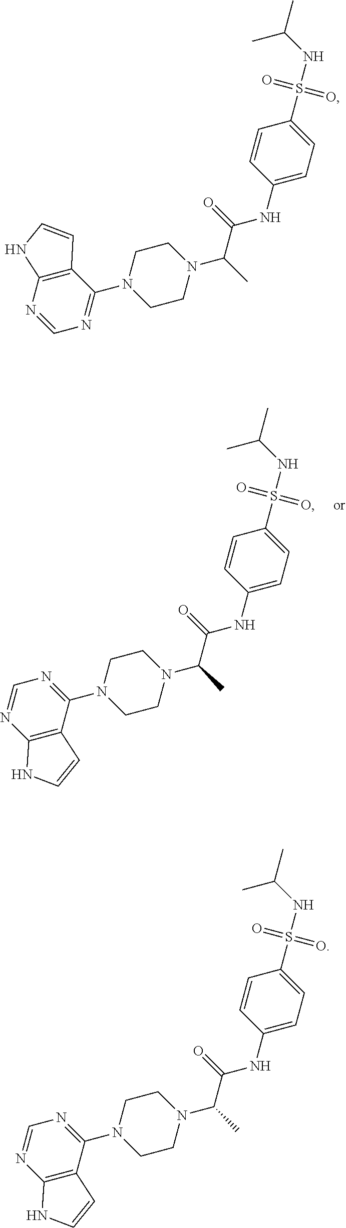 [SMILES: CC(C)NS(=O)(=O)c1ccc(NC(=O)C(C)N2CCN(c3ncnc4[nH]ccc34)CC2)cc1 ; CC(C)NS(=O)(=O)c1ccc(NC(=O)[C@@H](C)N2CCN(c3ncnc4[nH]ccc34)CC2)cc1 ; CC(C)NS(=O)(=O)c1ccc(NC(=O)[C@H](C)N2CCN(c3ncnc4[nH]ccc34)CC2)cc1]

In some embodiments, the compound of Formula I is

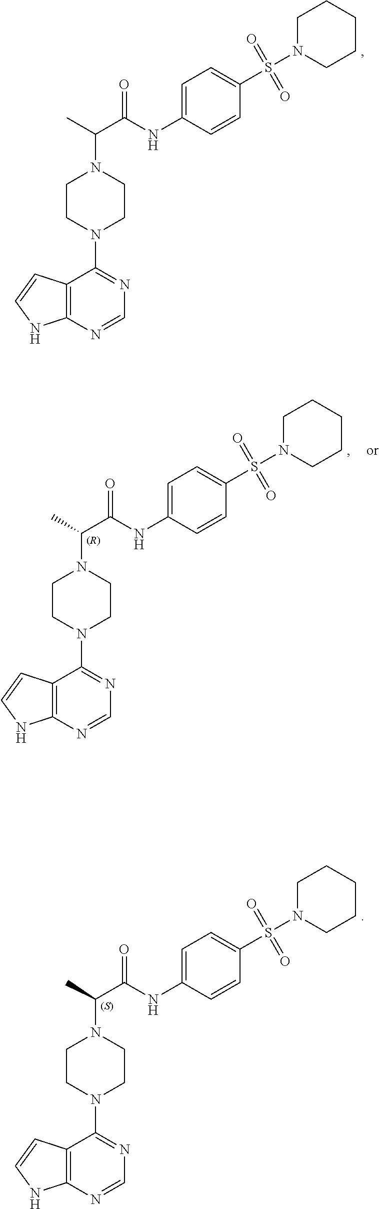

In some embodiments, R₂ and R₃, together with the included N atom, form a 6-membered ring, optionally substituted with C₁-C₄alkyl, C₇ to C₁₂ aralkyl, or —C(O)—NR₆R₇.

Accordingly, in some embodiments, the compound of Formula I has a structure according to Formula II

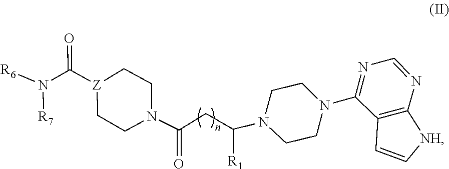

wherein Z is C or N.

In some embodiments, Z is C. In some embodiments, Z is N.

In some embodiments, n is 1. In some embodiments, n is 0.

In some embodiments, R1 is H.

In some embodiments, R₆ is H. In some embodiments, R₆ is C₁-C₄ alkyl.

In some embodiments, R₇ is H. In some embodiments, R₇ is C₁-C₄ alkyl. In some embodiments, R₇ is phenyl.

In some embodiments, R₆ and R₇, together with the included N atom, form a 6-membered ring which may optionally be substituted with C₁-C₄ alkyl, and wherein said 6-membered ring optionally includes one additional N atom.

In some embodiments, the compound of Formula II has a structure selected from the group consisting of

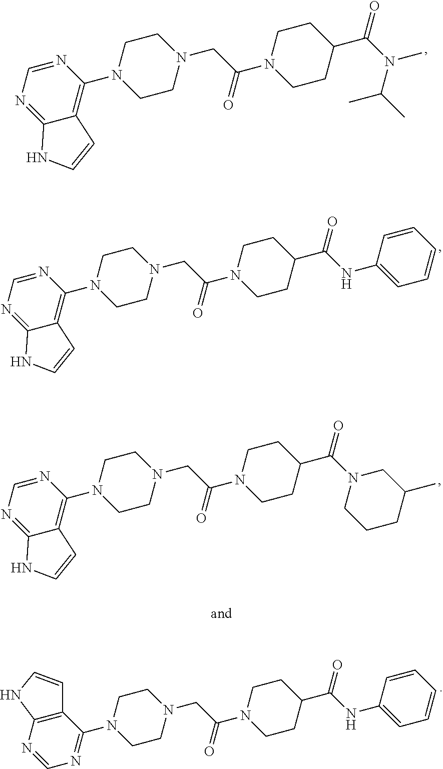 [SMILES: CC(C)N(C)C(=O)C1CCN(C(=O)CN2CCN(c3ncnc4[nH]ccc34)CC2)CC1 ; O=C(Nc1ccccc1)C1CCN(C(=O)CN2CCN(c3ncnc4[nH]ccc34)CC2)CC1 ; CC1CCCN(C(=O)C2CCN(C(=O)CN3CCN(c4ncnc5[nH]ccc45)CC3)CC2)C1 ; O=C(Nc1ccccc1)C1CCN(C(=O)CN2CCN(c3ncnc4[nH]ccc34)CC2)CC1]

In some embodiments, the compound of Formula I has a structure:

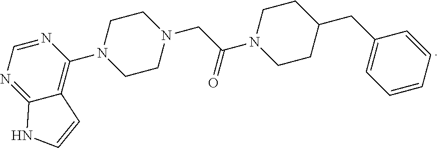 [SMILES: O=C(CN1CCN(c2ncnc3[nH]ccc23)CC1)N1CCC(Cc2ccccc2)CC1]

### Pharmacological Profile of Compounds of the Disclosure

Compounds of Formula I generally possess inhibitory activity toward one or more Janus Kinase (JAK) subtypes. The effect of compounds on the JAK enzyme and/or animal can be determined or measured. Methods for determining JAK activity include those described in the Examples of the present disclosure as well as those disclosed in WO1999/65908, WO1999/65909, WO2001/42246, WO2002/00661, WO2002/096909, WO2004/046112 or WO2007/012953, the content of each of which is incorporated herein by reference.

In some embodiments, a compound of Formula I has a JAK1 activity, such as binding affinity, as measured by enzyme binding IC₅₀, of less than about 1 micromole, such as less than about 500 nM, less than 250 nM, less than 100 nM, or less than 50 nM. In some embodiments, a compound of Formula I binds to JAK1 with an IC₅₀ between about 25 and about 100 nM, between about 100 and about 250 nM, between about 250 and about 500 nM, or between about 500 and about 1000 nM. In some embodiments, the compound of Formula I has human JAK1 activity, canine JAK1 activity, or both.

In some embodiments, a compound of Formula I exhibits binding selectivity toward JAK-1 relative to other enzymes, such as other JAK subtypes. In some embodiments, a compound of Formula I has selectivity for JAK-1 over JAK-2 on the order of about 5-fold or greater, or about 10-fold or greater, such as from about 5 or about 10 to about 20, about 50, or about 100-fold. In some embodiments, the compound of Formula I is selective for human JAK-1. In some embodiments, the compound of Formula I is selective for canine JAK-1. In some embodiments, the compound of Formula I is selective for both human and canine JAK-1.

In some embodiments, a compound of Formula I is selective toward functional inhibition of JAK-1 (canine, human, or both). In some embodiments, a compound of Formula I has selectivity for JAK-1 inhibition over JAK-2 inhibition on the order of about 5-fold or greater, or about 10-fold or greater, such as from about 5 or about 10 to about 20, about 50, or about 100-fold.

### Preparation of Compounds of the Disclosure

Compounds of the present disclosure may be prepared by methods known in the art of organic synthesis as set forth in part by the synthetic schemes in the following description, examples, and Figures in conjunction with the guidance provided herein. In the schemes described below and provided in the Figures, it is understood that protecting groups for sensitive or reactive groups may be employed where necessary in accordance with general principles or chemistry in accordance with the guidance provided herein. Protecting groups may be manipulated according to standard methods of organic synthesis (T. W. Greene and P. G. M. Wuts, “Protective Groups in Organic Synthesis,” Third edition, Wiley, New York 1999). These groups may be removed at a convenient stage of the compound synthesis using methods that are readily apparent to those skilled in the art based on the detailed teaching provided herein. The selection processes, as well as the reaction conditions and order of their execution, shall be consistent with the present disclosure.

Generally, the methods of preparing compounds of the present disclosure comprise combinations of reactions and conditions. Schemes 1 and 2 illustrate representative, non-limiting alternative strategies for preparation of compounds of Formula (I). Each aspect of the preparative methods according to Schemes 1 and 2 are discussed further herein below. The reactions in the Schemes can be performed through chemical reactions using standard synthetic chemistry procedures and practices as known in the art or described herein.

With reference to exemplary Scheme 1, in some embodiments, compounds of the disclosure (e.g., compounds of Formula I) may be prepared by amidation of a common intermediate carboxylic acid (4). With continued reference to Scheme 1, in some embodiments, 4-chloro-7H-pyrrolo[2,3-d]pyrimidine 1 is allowed to react with a piperazine 2 to form the ester intermediate 3. In some embodiments, n is zero. In some embodiments, n is 1. In some embodiments, R₁ is H. In some embodiments, R₁ is C₁-C₃ alkyl, which may optionally be substituted. In some embodiments, R₁ is methyl. In particular embodiments, R₁ is H and n is 0. Compounds of structure 2 may be commercially available, or may be readily obtained according to known methods. Suitable conditions for alkylation of 4-chloro-7H-pyrrolo[2,3-d]pyrimidine 1 with piperazine 2 include, but are not limited to, reaction in the presence of a non-nucleophilic amide base and in a polar solvent, generally at elevated temperature. For example, the reaction may conveniently be conducted in n-butanol in the presence of diisopropylethylamine at about 90° C. With continued reference to Scheme 1, the ester 3 may be saponified to provide the acid 4. Suitable conditions include, but are not limited to, reaction with an alkali metal hydroxide in an aqueous alcohol solvent (e.g., lithium hydroxide in aqueous methanol). With continued reference to Scheme 1, the acid 4 is then coupled with the desired amine 5 to provide the compound of Formula I. One of skill in the art will recognize appropriate coupling conditions. Suitable coupling conditions include, but are not limited to, those used for peptide formation (e.g., HATU, TBTU, BOP, PyBOP, PyBROP, and the like) in the presence of an amine base (e.g., pyridine, triethyl amine, diisopropylethylamine, and the like). Amine components 5 may be commercially available or may be readily obtained by standard synthetic procedures such as those described further herein below.

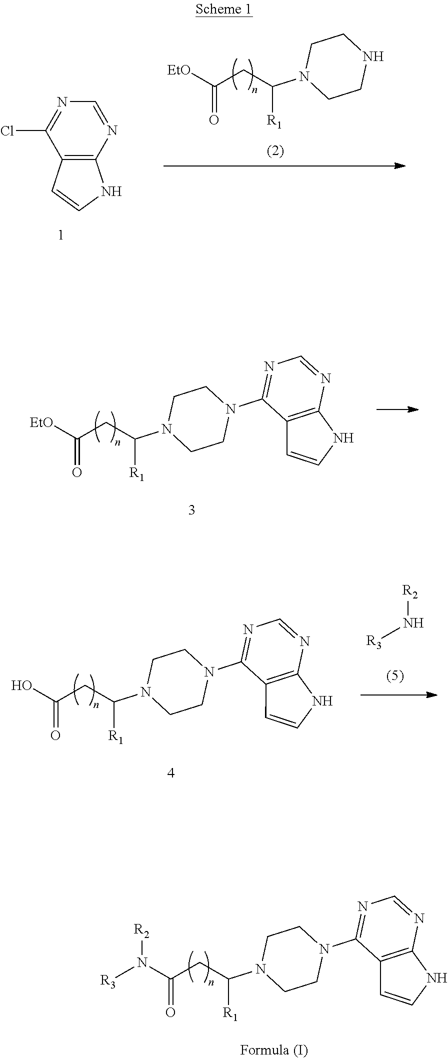

Scheme 2 provides an exemplary alternative synthetic scheme for compounds of Formula I. With reference to Scheme 2, in some embodiments, compounds of the disclosure (e.g., compounds of Formula I) may be prepared by alkylation of piperaine intermediate 6 with a chloro amide 7. Chloro amide 7 may in turn be prepared from amine 5 and acid chloride 8. Generally, piperazine intermediate 6 may be prepared by reaction of 4-chloro-7H-pyrrolo[2,3-d]pyrimidine 1 with piperazine as described above with respect to Scheme 1 and intermediate 3. Suitable reaction conditions include, but are not limited to, reaction in an aprotic solvent in the presence of an amine base such as pyridine, and optionally a catalyst such as N,N-dimethylaminopyridine. In some embodiments, R₁ is H and n is zero (i.e., 8 is chloroacetyl chloride). In some embodiments, the alkylation of 6 with chloride 7 is performed in a polar aprotic solvent such as DMF in the presence of an amine base such as triethylamine, optionally in the presence of a catalytic or stoichiometric amount of sodium or potassium iodide.

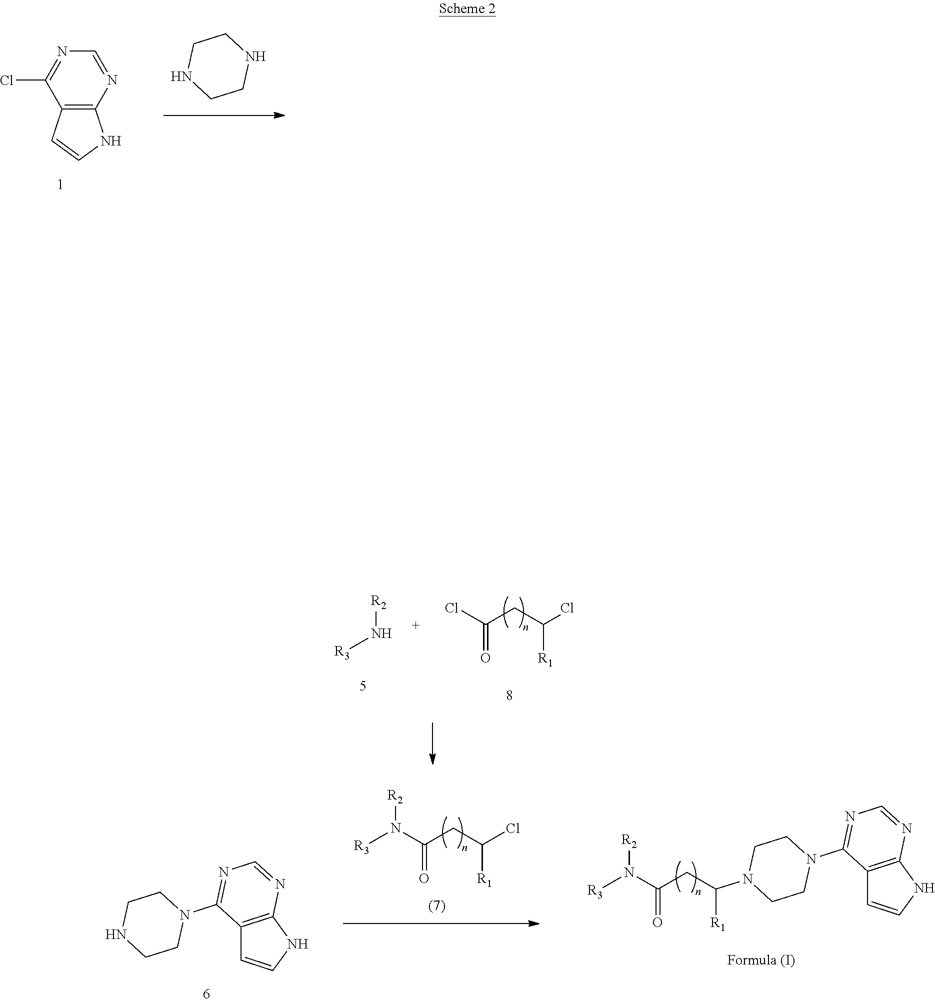

With continued reference to Scheme 1 and Scheme 2, in some embodiments, amine 5 is a primary amine. In some embodiments, amine 5 is a secondary amine. In some embodiments, amine 5 is a primary or secondary alkyl amine, benzylic amine, aryl amine, or heterocycloalkylamine. In some embodiments, R₂ is H or CH₃. In some embodiments, R₃ is C₁-C₄ alkyl or phenyl. In some embodiments, R₃ is:

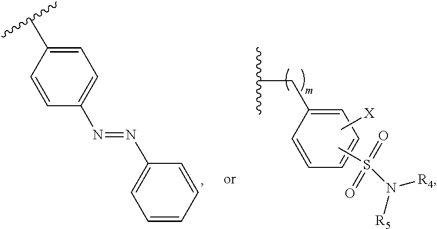

wherein:
m is 0 or 1;
X is H, F, Cl, OCH₃, or CH₃;
R₄ is H or CH₃,
R₅ is H, C₁-C₄ alkyl, C₃-C₅ cycloalkyl, CH₂CH₂OCH₃, or 2-pyrimidinyl; or
R₄ and R₅, together with the included N atom, form a 4-, 5- or 6-membered ring, wherein said 6-membered ring optionally includes one additional heteroatom selected from N and O; or
R₂ and R₃, together with the included N atom, form a 6-membered ring, optionally substituted with C₁-C₄alkyl, C₇ to C₁₂ aralkyl, or —C(O)—NR₆R₇,
wherein:
R₆ is H or C₁-C₄ alkyl;
R₇ is H, C₁-C₄ alkyl, phenyl; or
R₆ and R₇, together with the included N atom, form a 6-membered ring which may optionally be substituted with C₁-C₄alkyl, and wherein said 6-membered ring optionally includes one additional N atom.

In some embodiments, R₂ is H and R₃ is

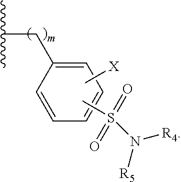

wherein each of m, X, R₄, and R₅ are as defined herein above.

In some embodiments, m is 1.

In some embodiments, m is 1, X is H, and amine 5 is selected from the group consisting of

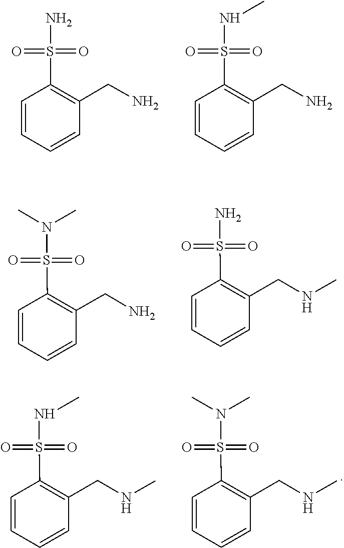

Such amines are generally either commercially available, or may be readily prepared according to methods known to one of skill in the art.

In some embodiments, m is 0. Accordingly, in some embodiments, the amine 5 has a structure 9:

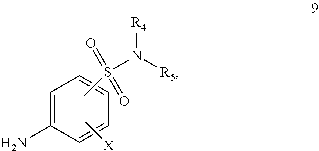

where X, R₄, and R₅ are each as described herein above. Such amines may be commercially available or may be readily prepared according to known methods. For example, sulfonamido amines 9 may be conveniently prepared from a corresponding nitrobenzenesulfonyl chloride 10 according to Scheme 3. With reference to Scheme 3, generally, a commercially available nitrobenzensulfonyl chloride 10 is allowed to react with an amine 11 in the preence of a suitable base, such as pyridine or a non-nucleophilic amine, to form a sulfonamide 12. Such sulfonamides may readily be reduced to amino sulfonamides 9 by, for example, hydrogenation over a suitable catalyst or reduction with iron, zinc, and the like under acidic conditions.

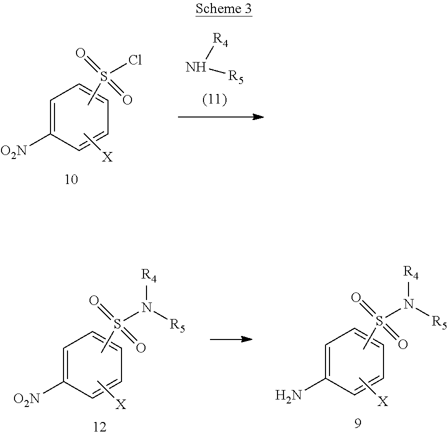

In some embodiments, sulfonamido amine 9 is selected from the group consisting of:

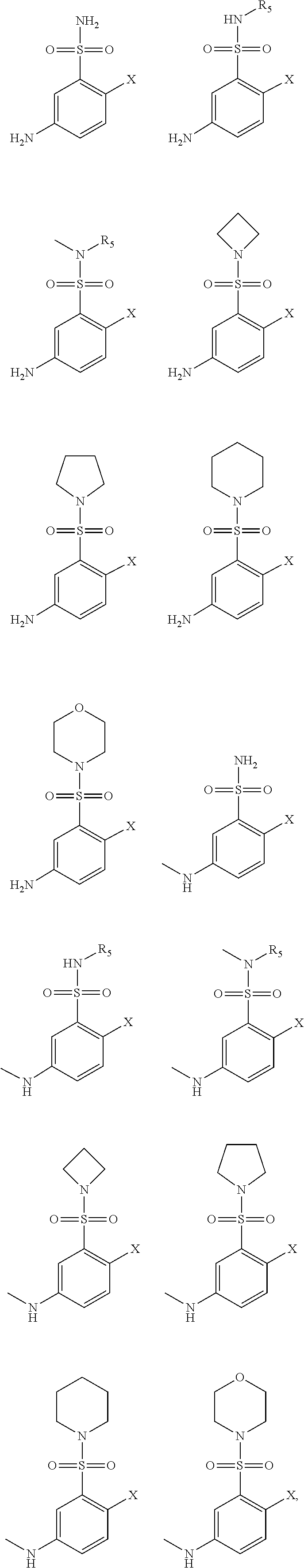

wherein:
X is H, F, Cl, OCH₃, or CH₃; and
R₅ is C₁-C₄ alkyl, C₃-C₅ cycloalkyl, CH₂CH₂OCH₃, or 2-pyrimidinyl.

In some embodiments, sulfonamido amine 9 is selected from the group consisting of

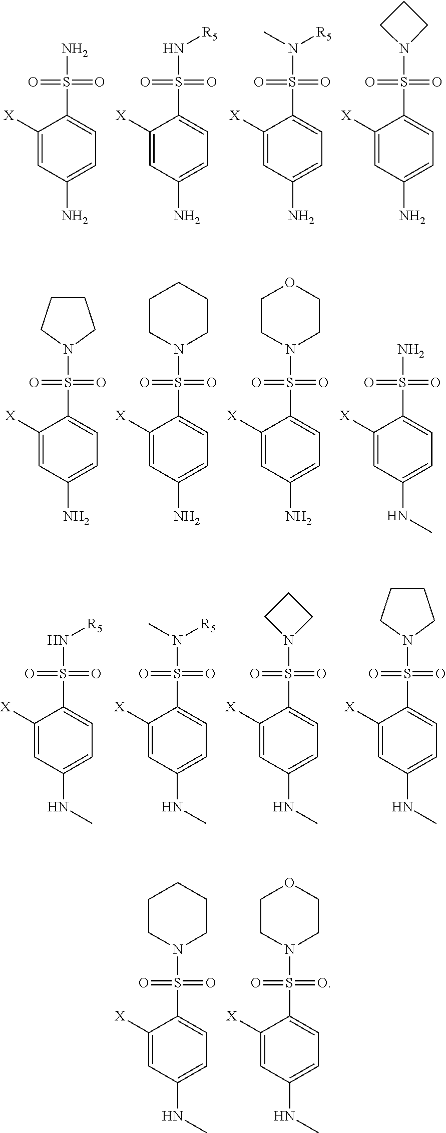

In some embodiments, sulfonamido amine 9 is selected from the group consisting of

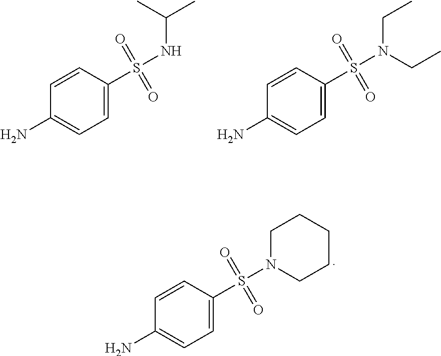 [SMILES: CC(C)NS(=O)(=O)c1ccc(N)cc1 ; CCN(CC)S(=O)(=O)c1ccc(N)cc1 ; Nc1ccc(S(=O)(=O)N2CCCCC2)cc1]

In some embodiments, amine 5 is:

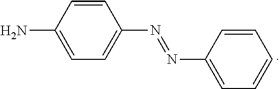 [SMILES: Nc1ccc(/N=N/c2ccccc2)cc1]

In some embodiments, the compound of Formula I has a structure according to Formula II:

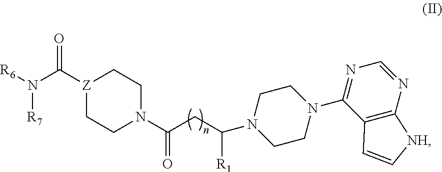

wherein Z is C or N, and each of R₁, R₆, R₇, and n are as described herein above. Accordingly, in some embodiments, the amine 5 has a structure according to Formula III:

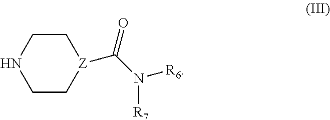

In some embodiments, Z is C. Such amines may be prepared by coupling N-protected piperidine-4-carboxylic acid with an amine HNR₆R₇ according to methods known to one of skill in the art.

In some embodiments, amine 5 is:

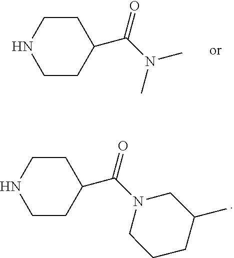

In some embodiments, amine 5 is:

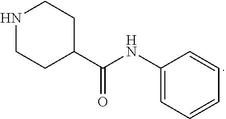 [SMILES: O=C(Nc1ccccc1)C1CCNCC1]

In some embodiments, Z is N. Such amines may be prepared by coupling mono-N-protected piperazine with an amine HNR₆R₇ and a phosgene equivalent according to methods known to one of skill in the art.

In some embodiments, amine 5 is:

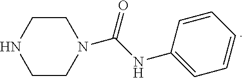 [SMILES: O=C(Nc1ccccc1)N1CCNCC1]

In some embodiments, the compound of Formula I has a structure according to Formula IV:

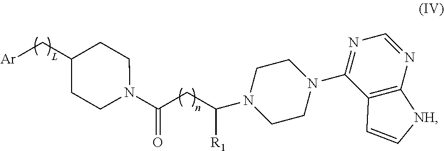

where Ar is phenyl or substituted phenyl, and L is an integer from 1 to 6. Accordingly, in some embodiments, the amine 5 has a structure:

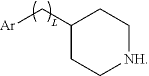

Such amines may be prepared from a coupling reaction between an aryl halide and a piperidinyl alkene according to exemplary Scheme 4. With reference to Scheme 4, an aryl bromide is coupled with a suitable piperidinyl alkene (13, 14, or 15) with palladium catalysis (e.g., Heck conditions). One of skill in the art will recognize that piperidine alkene 13 will provide an amine 5 where L is 1; piperidine alkene 14 will provide an amine 5 where L is 2, and a piperidine alkene 15 where p is 1, 2, 3, or 4 will provide an amine 5 where L is 3, 4, 5, or 6, respectively. One of skill in the art will also recognize that suitable protecting groups may be utilized on the nitrogen of the corresponding piperidine alkene (e.g., BOC).

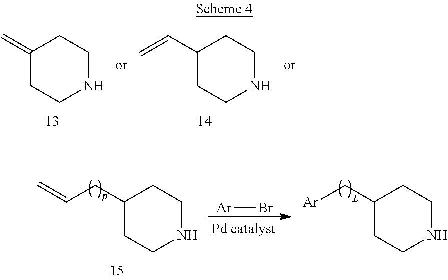

In some embodiments, Ar is phenyl and L is 1. Accordingly, in some embodiments, the amine 5 is:

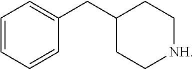 [SMILES: c1ccc(CC2CCNCC2)cc1]

### Isotopes and Isotopically Labeled Compounds

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. Accordingly, reference to a certain element is meant to include all isotopes of that element. For example, if an R group is defined to include hydrogen or H, it also includes isotopes thereof. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically enriched compounds may be prepared by conventional techniques well known to those skilled in the art.

The compounds described herein further include all pharmaceutically acceptable isotopically labeled compounds. An “isotopically” or “radio-labeled” compound may be a compound where one or more atoms are replaced or substituted by an atom having an atomic mass or mass number different from the atomic mass or mass number typically found in nature (i.e., naturally occurring). For example, in some embodiments, in the compounds described herein hydrogen atoms are replaced or substituted by one or more deuterium or tritium.

Certain isotopically labeled compounds of this disclosure, for example, those incorporating a radioactive isotope, may be useful in drug and/or substrate tissue distribution studies. The radioactive isotopes tritium, i.e., ³H, and carbon 14, i.e., ¹⁴C, may be particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. In some embodiments, the compound comprises at least one deuterium atom. For example, one or more hydrogen atoms in a compound of the present disclosure can be replaced or substituted by deuterium. In some embodiments, the compound comprises two or more deuterium atoms. In some embodiments, the compound comprises 1, 2, 3, 4, 5, 6, 7, 8, 9, 10, 11 or 12 deuterium atoms. Suitable isotopes that may be incorporated in compounds described herein include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, 123I, ¹²⁴J ¹²⁵I, and ¹³¹I. Substitution with positron emitting isotopes, such as ¹¹C, ¹⁸F, O, and ¹³N, can be useful in Positron Emission Topography (PET) studies.

Isotopically labelled versions of the compounds disclosed herein can generally be prepared by following procedures analogous to those disclosed in the Schemes and/or in the Examples herein, by substituting an appropriate isotopically labelled reagent for a non-isotopically labelled reagent.

### Isomers

In some embodiments, compounds of the disclosure may be enriched to provide predominantly one enantiomer of a compound described herein. An enantiomerically enriched mixture may comprise, for example, at least 60 mol percent of one enantiomer, or at least 75, at least 80, at least 85, at least 90, at least 95, at least 96, at least 97, at least 98, at least 99, at least 99.5 or even 100 mol percent. In some embodiments, the compounds described herein enriched in one enantiomer may be substantially free of the other enantiomer, wherein substantially free may mean that the substance in question makes up less than 10%, or less than 5%, or less than 4%, or less than 3%, or less than 2%, or less than 1% as compared to the amount of the other enantiomer, e.g., in the compound mixture. For example, if a compound mixture contains 98 grams of a first enantiomer and 2 grams of a second enantiomer, it would be said to contain 98 mol percent of the first enantiomer and only 2 mol percent of the second enantiomer.

In some embodiments, the compounds of the disclosure may be enriched to provide predominantly one diastereomer of a compound disclosed herein. A diastereomerically enriched mixture may comprise, for example, at least 60 mol percent of one diastereomer, or at least 75, at least 80, at least 85, at least 90, at least 95, at least 96, at least 97, at least 98, at least 99, at least 99.5, or even 100 mol percent. In some embodiments, the compounds described herein enriched in one diastereomer may be substantially free of other diastereomers, wherein substantially free may mean that the substance in question makes up less than 10%, or less than 5%, or less than 4%, or less than 3%, or less than 2%, or less than 1% as compared to the amount of other diastereomers, e.g., in the compound mixture.

Diastereomeric mixtures can be separated into their individual diastereomers on the basis of their physical chemical differences by methods well known to those skilled in the art, such as, for example, by chromatography and/or fractional crystallization. Enantiomers may be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column. Also, some of the compounds of the disclosure may be atropisomers or rotameric forms and are considered as part of this disclosure.

### Metabolites

The disclosure herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure may include compounds produced by a process comprising administering a compound of this disclosure to a subject, e.g., a mammal, for a period of time sufficient to yield a metabolic product thereof. Such products are typically identified by administering a radiolabeled compound of the disclosure in a detectable dose to subject, such as rat, mouse, guinea pig, monkey, or to human, allowing sufficient time for metabolism to occur, and isolating its conversion products from the urine, blood or other biological samples.

### Salts and Solvates

The present disclosure further provides pharmaceutically acceptable salts, solvates (e.g., hydrates), and combinations thereof of any of the compounds as disclosed herein. The use of the terms “salt,” “hydrate,” “solvate,” and the like, is intended to equally apply to the salt, hydrate, or solvate of enantiomers, diastereomers, isomers, stereoisomers, rotamers, tautomers, positional isomers, or racemates of the disclosed compounds.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. In some embodiments, the “pharmaceutically acceptable salts” may include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, 1,1-methene-bis-2-hydroxy-3-naphthoate, einbonate, pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

Suitable anionic salt forms include, but are not limited to acetate, benzenesulfonate, benzoate, benzylate, bicarbonate, bitartrate, bitartrate, bromide, calcium edetate, camsylateh, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionatei, lactate, lactobionate, malate, maleate, mandelate, mesylate, methylbromide, methylnitrate, methylsulfate, mucate, napsylate, nitrate, pamoate (embonate), pantothenate, phosphate and diphosphate, polygalacturonate, salicylate and disalicylate, stearate, subacetate, succinate, sulfate, tannate, tartrate, teoclate, tosylate, triethiodide, valerate, and the like.

Suitable cationic salt forms include, but are not limited to aluminum, benzathine, calcium, ethylene diamine, lysine, magnesium, meglumine, potassium, procaine, sodium, tromethamine, zinc, and the like. Suitable cationic salt forms include, but are not limited to benzathine, chloroprocaine, choline, diethanolamine, ethylenediamine, meglumine, procaine, aluminum, calcium, lithium, magnesium, potassium, sodium, zinc, and the like.

In some embodiments, the salt is selected from the group consisting of acetate, ascorbate, aspartate, benzoate, besylate, bicarbonate/carbonate, bisulfate/sulfate, borate, camsylate, citrate, edisylate, etoglutarate, esylate, formate, fumarate, gluceptate, gluconate, glucuronate, glycerophosphate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, and trifluoroacetate.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Compounds of the disclosure, including their stereoisomers and tautomers, as well as salts of any thereof, may exist as solvates. Often, crystallizations produce a solvate of the compound of the disclosure. As used herein, the term “solvate” may refer to an aggregate that comprises one or more molecules of a compound of the disclosure with one or more molecules of solvent. The solvent may be water, in which case the solvate may be a hydrate.

Alternatively, the solvent may be an organic solvent. Thus, the compounds and salts of the present disclosure may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases the compound of the disclosure may merely retain adventitious water or be a mixture of water plus some adventitious solvent.

### Pharmaceutical Formulations and Compositions

A compound of the present disclosure, as well as its salts, solvates, isomers, and the like, can be administered to the subject by itself or in a pharmaceutical composition where it is mixed with one or more biologically suitable and pharmaceutically acceptable carriers or excipients. The compositions can be in various forms, including, but not limited to, oral formulations, injectable formulations, suppository formulations, and topical, dermal, or subdermal formulations. Selection of an appropriate formulation of a compound as provided herein may be based, e.g., on the physicochemical properties of the compound (and any other optional active agent to be administered), the type of animal being treated, the condition of the animal being treated, and cost.

Pharmaceutical compositions can comprise at least the compounds or salts described herein and one or more pharmaceutically acceptable carriers, diluents, excipients, stabilizers, dispersing agents, suspending agents, and/or thickening agents. The particular components and the relative amounts of each will vary depending on, for example, the intended route of administration. The compositions can be formulated to contain a single daily dose or a convenient fraction of a daily dose in a dosage unit (e.g., a single tablet, single capsule, convenient volume of liquid/ointment, etc.). The amount of the compound of the formula provided above included within a given composition for use in animal and human health can vary widely. The compound is typically included within a composition in an amount likely to induce the desired effect. Compositions that will be administered to a subject or patient commonly take the form of one or more dosage units, where for example, a tablet may be a single dosage unit, and a container of one or more compounds of the disclosure, or pharmaceutically acceptable salts, solvates, stereoisomers, or tautomers thereof, in aerosol form may hold a plurality of dosage units.

Pharmaceutical compositions for use in accordance with the present disclosure may be formulated in a conventional manner using one or more pharmaceutically acceptable carriers comprising excipients and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991). In general, pharmaceutical compositions of the disclosed compounds may be manufactured in a manner that is itself known, e.g., by means of conventional mixing, dissolving, granulating, dragee-making, levigating, emulsifying, encapsulating, entrapping or lyophilizing processes. The techniques for formulation may be found in references well known to one of ordinary skill in the art, such as “Remington's Pharmaceutical Sciences,” Mack Publishing Co., Easton, PA, latest edition.

### Oral Dose Forms

The compositions described herein can be formulated for oral administration. When intended for oral administration, pharmaceutical compositions of the present disclosure typically are either solid or liquid form, where semi solid, semi liquid, suspension and gel forms may be included within the forms considered herein as either solid or liquid.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. When the pharmaceutical composition is in the form of a capsule, for example, a gelatin capsule, it may contain, in addition to materials disclosed herein, a liquid carrier such as polyethylene glycol or oil.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. In some embodiments, a capsule (a hard capsule or a soft capsule), e.g., a gelatin capsule comprising a compound according to a formula as described herein is provided. A hard gelatin capsule, in some embodiments, can a compound according to a formula as described herein admixed with an inert solid diluent, e.g., a starch, powdered cellulose (e.g., crystalline or microcrystalline cellulose), a sugar (e.g., fructose, mannitol, or sucrose), a grain flour, calcium carbonate, calcium phosphate, and/or kaolin, enclosed in a capsule. A soft or liquid gelatin capsule, in some embodiments, can comprise a compound according to a formula as described herein mixed with water or a solvent such as propylene glycol, polyethylene glycol (PEG), and/or ethanol or mixed with an oil medium, e.g., peanut oil, liquid paraffin, or olive oil, enclosed within a capsule. In some embodiments, such capsules are microcapsules.

In some embodiments, a composition in tablet form is provided, comprising a compound according to a formula as described herein in combination with one or more pharmaceutically acceptable excipients. Tablets are generally prepared via direct compression (e.g., wet granulation or dry granulation of ingredients). Excipients known to be suitable for the manufacture of tablets are generally known and include, for example, inert diluents (e.g., starches, lactose, mannitol, powdered sugar, powdered cellulose derivatives, kaolin, calcium carbonate, sodium carbonate, lactose, calcium phosphate, calcium sulfate, sodium phosphate, and/or inorganic salts such as sodium chloride); granulating and disintegrating agents (e.g., starch such as corn or potato starch, clay, cellulose, methylcellulose, carboxymethyl cellulose, algins, and/or alginic acid, agar, bentonite, wood cellulose, powdered natural sponge, cation-exchange resins, guar gum, citrus pulp, and/or sodium lauryl sulfate); binding agents (e.g., starch, gelatin, sugars (e.g., lactose, fructose, glucose, and the like), natural or synthetic gums such as acacia, alginates, methylcellulose, polyvinylpyrrolidine, and the like), polyethylene glycol, ethyl cellulose, and/or waxes; and lubricating agents, (e.g., magnesium stearate, calcium stearate, stearic acid, hydrogenated vegetable oil, and/or talc). Tablets may be uncoated or may be coated by known techniques (which can serve to delay disintegration and absorption in the gastrointestinal tract and thereby provide for sustained action over a longer period of time).

For example, in some embodiments, a time delay material such as glyceryl monostearate or glyceryl distearate may be employed. Tablets may also be optionally coated by techniques described in U.S. Pat. No. 4,256,108 to Theeuwes; U.S. Pat. No. 4,166,452 to Generales Jr.; and U.S. Pat. No. 4,265,874 to Bonsen et al., which are all incorporated herein by reference with respect to the preparation of osmotic therapeutic tablets for controlled release. Troches are an example of tablets, and are typically formulated as small, hard tablets which dissolve slowly when placed under the tongue. Tablets can optionally be coated, e.g., with sugar as a flavorant and sealant or with film-forming protecting agents to modify the dissolution properties of the tablet.

Pharmaceutical compositions of the disclosure may be in the form of a liquid, for example, an elixir, syrup, solution, emulsion or suspension. The liquid may be for oral administration or for delivery by injection, as two examples. When intended for oral administration, pharmaceutical compositions of the disclosure typically contain, in addition to one or more compounds of the disclosure, or pharmaceutically acceptable salts, solvates, stereoisomers, or tautomers thereof, one or more of a sweetening agent, preservatives, dye/colorant and flavor enhancer.

In some embodiments, a composition comprising a compound according to a formula as described herein is provided in the form of an emulsion, e.g., in the form of an oil-in-water or a water-in-oil emulsion. The oily phase of the emulsion may be a vegetable oil, e.g., olive oil or arachis oil or may be a mineral oil, e.g., liquid paraffin, or a mixture of any such oils. In some embodiments, the oily phase is formed from unsaturated polyglycosylated glycerides, triglycerides, (e.g., medium-chain triglycerides, such as C8-C10 caprylic/capric triglycerides), or combinations thereof. The aqueous phase can comprise water or glycol derivatives (e.g., propylene glycol, glycol ether, polyethylene glycol, or glycerol). Specific examples include, but are not limited to, propylene glycol, diethylene glycol monoethyl ether, dipropylene glycol monomethyl ether, and mixtures thereof. Suitable emulsifying agents may include, e.g., naturally occurring phosphatides, e.g., soybean, lecithin, and esters or partial esters derived from fatty acids and hexitol anhydrides, e.g., sorbitan monoleate, and condensation products of the referenced partial esters with ethylene oxide, e.g., polyoxyethylene sorbitan monooleate. The emulsions may also optionally contain sweetening agents, bittering agents, flavoring agents, and/or preservatives. In one embodiment, the emulsion is in the form of a microemulsion (composed of stable dispersions of microdroplets of the aqueous phase in the oily phase or of microdroplets of the oily phase in the aqueous phase). Microemulsions are quaternary systems comprising an aqueous phase, an oily phase, a surfactant and a co-surfactant. They are translucent and isotropic liquids. The size of these microdroplets is less than 200 nm (in contrast to microdroplets with sizes of about 1000 nm to 100,000 nm for emulsions). In some embodiments, the oily phase will represent a % v/v range selected from the group consisting of about 2 to about 15%; about 7 to about 10%; and about 8 to about 9% v/v of the microemulsion. Generally, the aqueous phase will represent a proportion from about 1 to about 4% v/v in the microemulsion. The interfacial film is composed of an alternation of surface-active (SA) and co-surface-active (Co-SA) molecules which, by lowering the interfacial tension, allows the microemulsion to be formed spontaneously. Surfactants for the microemulsion include diethylene glycol monoethyl ether, dipropylene glycol monomethyl ether, polyglycolyzed C8-C10 glycerides or polyglyceryl-6 dioleate. In addition to these surfactants, the co-surfactants include short-chain alcohols, such as ethanol and propanol. Some compounds are common to the three components discussed above, i.e., aqueous phase, surfactant and co-surfactant. However, it is well within the skill level of the practitioner to use different compounds for each component of the same formulation. In one embodiment, the co-surfactant to surfactant ratio will be from about 1/7 to about ½. In another embodiment, there will be from about 25 to about 75% v/v of surfactant and from about 10 to about 55% v/v of co-surfactant in the microemulsion.

In some embodiments, a composition comprising a compound according to a formula as described herein is provided in the form of a suspension, generally comprising the compound (optionally along with other ingredients) dispersed in a liquid. The compound is typically in the form of a dispersible powder or granule; dispersible powders and granules suitable for preparation of a suspension generally provide the active ingredient in admixture with a dispersing or wetting agent, suspending agent and one or more preservatives. Suitable dispersing or wetting agents and suspending agents are exemplified by those already mentioned above. Additional excipients, for example, sweetening, bittering, flavoring, and coloring agents, may also be present. The liquid can be oily or aqueous. Oily suspensions may be formulated by suspending the compound in a vegetable oil, for example, atachis oil, olive oil, sesame oil or coconut oil, or in mineral oil such as liquid paraffin. The oily suspensions may contain a thickening agent, for example, beeswax, hard paraffin or cetyl alcohol. Sweetening agents such as sucrose, saccharin or aspartame, bittering agents, and flavoring agents may be added to provide a palatable oral preparation. These compositions may be preserved by the addition of an antioxidant such as ascorbic acid, or other known preservatives. Aqueous suspensions contain the compound in admixture with excipients suitable for the manufacture of aqueous suspensions. For example, such aqueous suspensions may comprise excipients that are suspending agents, for example, sodium carboxymethylcellulose, methylcellulose, hydroxy-propylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example polyethylene sorbitan monooleate. Aqueous suspensions may also contain one or more preservatives, for example ethyl, or n-propyl, p-hydroxybenzoate, one or more coloring agents, one or more flavoring agents, and one or more sweetening agents and/or bittering agents, such as those set forth above. Aqueous suspensions may comprise, e.g., juice (such as apple or orange juice).

In some embodiments, a composition comprising a compound according to a formula as described herein is provided in the form of a syrup or elixir. Syrups and elixirs may be formulated with sweetening agents, for example, glycerol, propylene glycol, sorbitol, or sucrose. Such formulations may also contain a demulcent, a preservative, flavoring agent(s) and/or coloring agent(s).

In some embodiments, a composition comprising a compound according to a formula as described herein is in paste form. Examples of embodiments in a paste form include but are not limited to those described in U.S. Pat. No. 6,787,342 to Chen; U.S. Pat. No. 7,001,889 to Freehauf et al.; and U.S. Pat. No. 7,563,773 to Freehauf, each of which is incorporated herein by reference in its entirety. In addition to the compound(s) of the invention, the paste can also contain components including, e.g., fumed silica; a viscosity modifier (e.g., selected from PEG 200, PEG 300, PEG 400, PEG 600, monoethanolamine, triethanolamine, glycerol, propylene glycol, polyoxyethylene (20) sorbitan monooleate (polysorbate 80 or Tween 80), and polyoxamers (e.g., Pluronic L 81)); a carrier (e.g., a hydrophilic carrier selected from triacetin, a monoglyceride, a diglyceride, and a triglyceride); optionally, an absorbent (e.g., selected from magnesium carbonate, calcium carbonate, starch, and cellulose and its derivatives); and optionally, a colorant (e.g., selected from the group consisting of titanium dioxide iron oxide, and FD&C Blue #1 Aluminum Lake), stabilizer, surfactant, and/or preservative.

Sustained-release preparations can also be prepared. Examples of sustained-release preparations can include semipermeable matrices of solid hydrophobic polymers that can contain the compound or salt, and these matrices can be in the form of shaped articles (e.g., films or microcapsules). Examples of sustained-release matrices can include polyesters, hydrogels (e.g., poly(2-hydroxyethyl-methacrylate), or poly (vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(-)-3-hydroxybutyric acid.

### Injectable Forms

The compositions described herein can be formulated for administration as an injection. In a composition intended to be administered by injection, one or more of a surfactant, preservative, wetting agent, dispersing agent, suspending agent, buffer, stabilizer and isotonic agent may be included. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions described herein can be lyophilized or in powder form for reconstitution with a suitable vehicle, e.g., sterile pyrogen-free water, before use.

For parenteral administration, the compounds or salts can be formulated in a unit dosage injectable form (e.g., use letter solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Liquid pharmaceutical compositions of the disclosure, whether they be solutions, suspensions or other like form, may include one or more of the following adjuvants: sterile diluents such as water for injection, saline solution, physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. Parenteral preparations can be enclosed in ampoules, disposable syringes or multiple dose vials made of glass or plastic. In some embodiments, the adjuvant is physiological saline. In some embodiments, the injectable pharmaceutical composition is sterile.

### Other Dosage Forms

Pharmaceutical compositions of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration.

In some embodiments, a composition suitable for topical, dermal, and/or subdermal formulation comprising a compound according to the formula(s) described herein is provided. Topical, dermal, and subdermal formulations can include, e.g., emulsions, creams, ointments, gels, pastes, powders, patches, shampoos, pour-on formulations, ready-to-use formulations, spray formulations, and spot-on formulations. Such formulations can be, e.g., concentrated solutions, suspensions, microemulsions, or emulsions. Topical application can, in some embodiments, allow for the active compound(s) to be distributed through the glands (e.g., sebaceous glands) of the animal and/or allow the active compound(s) to achieve a systemic effect (plasma concentration) and/or allow for distribution throughout the haircoat. Certain suitable formulations for topical, dermal, and/or subdermal application include, but are not limited to, those disclosed in U.S. Pat. No. 6,395,765 to Etchegaray, which is incorporated herein by reference in its entirety.

Spot-on compositions are typically applied in a localized region which refers to an area other than the entire animal. Spot-on compositions are generally used by administering the composition at a particular location of an animal (e.g., between the shoulders). Another embodiment of a localized region is a stripe, e.g., a stripe from head to tail of the animal. Such compositions can be administered, e.g., via pipettes, squeeze-ons, or drop-ons.

The carrier can be a liquid carrier vehicle as described in U.S. Pat. No. 6,426,333, which is incorporated herein by reference in its entirety. For example, in one embodiment, a spot-on formulation comprises a solvent and a co-solvent wherein the solvent is selected from the group consisting of acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol, diethyl phthalate fatty acid esters, such as the diethyl ester or diisobutyl adipate, and a mixture of at least two of these solvents and the co-solvent is selected from the group consisting of absolute ethanol, isopropanol or methanol. The liquid carrier vehicle can optionally contain a crystallization inhibitor selected from the group consisting of an anionic surfactant, a cationic surfactant, a non-ionic surfactant, an amine salt, an amphoteric surfactant or polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, and acrylic derivatives, or a mixture of these crystallization inhibitors.

Pour-on compositions are generally used by pouring the composition along an animal's backline (e.g., from the neck to the tail). The pour-on formulations are advantageously oily, and generally comprise a diluent or vehicle and also a solvent (e.g., an organic solvent) for the active ingredient if the latter is not soluble in the diluent. Certain non-limiting pour-on compositions are disclosed, for example, in U.S. Pat. No. 6,010,710 to Etchegaray and U.S. Pat. No. 8,097,266 to Gogolewski et al., which are incorporated herein by reference in their entireties.

Spray-on compositions are generally used by spraying a composition along an animal's backline (e.g., from the neck to the tail). Each of these compositions can involve application, e.g., of a concentrated solution, suspension, microemulsion or emulsion.

Such topical compositions (e.g., spot-on, spray-on, and pour-on compositions) can generally comprise the compound as provided herein in combination with one or more diluents/vehicles and/or one or more solvents. Diluents/vehicles include, but are not limited to, plant oils (e.g., soybean oil, groundnut oil, castor oil, corn oil, cotton oil, olive oil, grape seed oil, sunflower oil, etc.); mineral oils (e.g., petrolatum, paraffin, silicone, etc.); aliphatic or cyclic hydrocarbons; medium-chain (such as C₈ to C₁₂) triglycerides, and combinations thereof. Solvents (e.g., organic solvents) that can be added in some embodiments include, but are not limited to, acetyltributyl citrate, fatty acid esters such as the dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol and diethyl phthalate, and mixtures of two or more thereof. In some embodiments, an emollient and/or spreading and/or film-forming agent is included in the composition. An emollient and/or spreading and/or film-forming agent can be, for example, selected from the group consisting of: polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, silicone oils, polydiorganosiloxane oils (such as polydimethylsiloxane (PDMS) oils), for example those containing silanol functionalities, or a 45V2 oil, anionic surfactants such as alkaline stearates, sodium, potassium or ammonium stearates; calcium stearate, triethanolamine stearate; sodium abietate; alkyl sulfates (e.g. sodium lauryl sulfate and sodium cetyl sulfate); sodium dodecylbenzenesulfonate, sodium dioctylsulphosuccinate; fatty acids (e.g. those derived from coconut oil), cationic surfactants such as water-soluble quaternary ammonium salts of formula N+R′R″R′″R″″, Y— in which the radicals R are optionally hydroxylated hydrocarbon radicals and Y— is an anion of a strong acid such as the halide, sulfate and sulfonate anions; cetyltrimethylammonium bromide is among the cationic surfactants which can be used, amine salts of formula N+R′R″R′″ in which the radicals R are optionally hydroxylated hydrocarbon radicals (e.g., octadecylamine hydrochloride), nonionic surfactants such as sorbitan esters, which are optionally polyoxyethylenated (e.g. polysorbate 80), polyoxyethylenated alkyl ethers; polyoxypropylated fatty alcohols such as polyoxypropylene-styrol ether; polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide, amphoteric surfactants such as the substituted lauryl compounds of betaine; and mixtures of at least two of these agents.

Pharmaceutical compositions of the disclosure may be intended for rectal administration, in the form, for example, of a suppository, which will melt in the rectum and release the drug. Compositions for rectal administration may contain an oleaginous base as a suitable nonirritating excipient. Such bases may include, without limitation, lanolin, cocoa butter and polyethylene glycol.

Pharmaceutical compositions of the disclosure may include various materials, which modify the physical form of a solid or liquid dosage unit. For example, the compositions may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

### Dosing

The dosage to be administered of a compound of Formula I as described herein will vary according to the particular compound, the subject and the physical condition thereof, the nature and severity of the disease, and the selected route of administration. Accordingly, the amount of compound or composition administered to an animal in need of treatment can vary widely. The amount of compound administered can depend, e.g., on such factors as the efficacy of the compound, the type of animal being treated, the animal's body weight, the animal's age, the desired effect, and the nature and severity of the disease. It is to be understood that the dosages may vary depending upon the requirements of each subject and the severity of the disorders or diseases being treated. One of skill in the art will be able to discern a specific efficacious dose. Generally, a therapeutically effective amount is the amount of compound or composition sufficient to provide a beneficial effect or to otherwise reduce a detrimental non-beneficial event to the subject to whom the compound or composition is administered. A therapeutically effective dose can be a dose that produces one or more desired or desirable (e.g., beneficial) effects for which it is administered, such administration occurring one or more times over a given period of time. Also, it is to be understood that an initial, higher dosage (i.e., one or more loading doses) may be administered in order to rapidly achieve the desired plasma concentration. On the other hand, the initial dosage may be smaller than the optimum and the daily dosage may be progressively increased during the course of treatment depending on the particular situation (i.e., dose titration).

Administration of the compounds or compositions provided herein may vary in frequency and duration. Administration may, for example, be intermittent in time and can be administered daily, weekly, biweekly, monthly, bimonthly, quarterly, or even for longer durations of time. In some embodiments, the administration is daily for a period of time of at least about one week, two weeks, three weeks, a month, or two months, and up to six months, a year, or multiple years, including for the lifetime of the subject.

In some embodiments, administration of the desired dose may be presented as a single dose or as divided doses administered at appropriate intervals, for example, as two, three, four or more sub-doses per day. The sub-dose itself may be further divided, e.g., into a number of discrete, loosely spaced administrations, such as multiple oral dose forms.

It will be recognized by one of skill in the art that the optimal quantity and spacing of individual dosages of a compound as described herein or a composition comprising the compound as described herein will be determined by the nature and extent of the condition being treated, the form, route and site of administration, and the age and condition of the particular subject being treated, and that a physician will ultimately determine appropriate dosages, frequency and treatment duration to be used. The selected dosage may be repeated as often as appropriate. If side effects develop the amount and/or frequency of the dosage can be altered or reduced, in accordance with normal clinical practice. One of skill in the art will be able to develop a specific administration protocol for a particular situation.

Method of Treating Canine Atopic Dermatitis and/or Pruritis

Provided herein is a method of treating canine atopic dermatitis and/or pruritis. The method generally comprises administering an effective amount of a compound of the disclosure, a salt, or solvate thereof, or a composition comprising the compound or salt or solvate thereof. Without wishing to be bound by any particular theory, it is believed that the potency and selectivity of the disclosed compounds in inhibiting Janus Kinase 1 (JAK-1; e.g., canine JAK-1 (cJAK-1)) provides efficacy and tolerability, which, together with a long half-life, allows for once-daily dosing.

The pathogenesis of canine atopic dermatitis is complex. Percutaneous sensitization to environmental allergens (e.g., dust mites, pollen, mold) and/or allergens from food induces skin infiltration by various inflammatory cells, activation of resident cells, and local production of inflammatory/itch mediators. Various factors can exacerbate canine atopic dermatitis, including ectoparasites, particularly fleas, environmental factors, cutaneous colonization/infection by bacteria and yeast, and epidermal barrier dysfunction.

The JAK/STAT signaling pathway has been implicated in the mediation of many abnormal immune responses such as allergies, asthma, and autoimmune diseases such as rheumatoid arthritis, JAK1 inhibition blocks the signaling of many important pro-inflammatory cytokines, including interleukin (IL)-2, IL-6, IL-7, and IL-15, which are known contributors to inflammatory disorders such as atopic dermatitis. Accordingly, inhibition of JAK signaling, and particularly JAK-1 signaling, has been proposed for treatment of immune-mediated disorders such as atopic dermatitis. In addition to the anti-inflammatory activity of JAK-1 inhibitors, it has been reported that type 2 cytokines, IL-4 and IL-13, directly stimulate sensory neurons and that chronic itch is dependent on neuronal IL-4Rα and JAK1 signaling (see, e.g., US Patent Application Publication No. 2021/0338812). Further, as discussed herein above, oclacitinib (APOQUEL®) has been approved by the FDA for treatment canine atopic dermatitis, lending further support to the concept of utilizing JAK inhibitors for the treatment of pruritis/immune/allergic disorders in companion animals. (Gonzales et al., J. Vet. Pharmacol Ther. 2014 August; 37(4):317-24).

Oclacitinib is most potent at inhibiting JAK-1 (IC₅₀=10 nM), and also inhibits the function of JAK1-dependent cytokines involved in allergy and inflammation (IL-2, IL-4, IL-6, and IL-13) as well as pruritus (IL-31) at IC₅₀'s ranging from 36 to 249 nM. (Gonzales et al., 2014). Oclacitinib has been characterized as a targeted therapy that selectively inhibits JAK1-dependent cytokines involved in allergy, inflammation, and pruritus with the suggestion that these are the mechanisms by which oclacitinib controls clinical signs associated with allergic skin disease in dogs.

It has now been found that the selectivity of oclacitinib toward cJAK-2, cJAK-3, and cJAK-4 relative to cJAK-1 is, in fact, relatively low. It is believed that this lack of selectivity is responsible for the dose limiting emesis observed in dogs and is likely cJAK-2 related. As noted herein above, it would be desirable in the art to provide a cJAK inhibitor with efficacy in treating canine atopic dermatitis and the related symptoms while allowing for the potential for once daily (QD) dosing.

Accordingly, disclosed herein are compounds having selectivity for JAK-1 over JAK-2 inhibition (canine, human, or both) on the order of 10-fold or greater, and methods of treating canine atopic dermatitis, pruritis, or both using such compounds. The methods of treatment generally comprise administering a therapeutically effective amount of the compound of Formula I, a salt or solvate thereof, or a composition comprising the compound, salt, or solvate.

Generally, the method comprises administering a compound of the present disclosure or its pharmaceutical compositions orally, parenterally, topically, rectally, or transmucosally. Parenteral adninistrations include indirect injections to generate a systemic effect or direct injections to the afflicted area. Topical administrations include the treatment of skin or organs readily accessible by local application, for example, ears. It also includes transdermal delivery to generate a systemic effect. The rectal administration includes the form of suppositories. In some embodiments, the route of administration is oral or parenteral. In specific embodiments, the route of administration is oral.

As described herein above, the frequency of dosing may vary based on the individual compound, the subject, the nature and severity of the dermatitis, and the like. In some embodiments, the compound or composition is administered orally on a daily basis. Also as described herein above, in some embodiments, the compound of the present disclosure has pharmacological and pharmacokinetic properties amenable to once daily dosing. Accordingly, in some embodiments, the administration is once daily.

### Combination Therapy

One or more compounds disclosed herein or pharmaceutically acceptable salts, solvates, stereoisomers, or tautomers of any of these, may be administered simultaneously with, prior to, or after administration of one or more other therapeutic agents. Such combination therapy may include administration of a single pharmaceutical dosage formulation that contains one or more compounds of the disclosure, or pharmaceutically acceptable salts, solvates, stereoisomers, or tautomers thereof, and one or more additional active agents, as well as administration of one or more compounds of the disclosure, or pharmaceutically acceptable salts, solvates, stereoisomers, or tautomers thereof, and each active agent in its own separate pharmaceutical dosage formulation. For example, one or more compounds of the disclosure, or pharmaceutically acceptable salts, solvates, stereoisomers, or tautomers thereof, and the other active agent can be administered to the subject together in a single oral dosage composition such as a tablet or capsule, or each agent administered in separate oral dosage formulations. Where separate dosage formulations are used, the one or more compounds of the disclosure, or pharmaceutically acceptable salts, solvates, stereoisomers, or tautomers thereof, and one or more additional active agents can be administered at essentially the same time, e.g., concurrently, or at separately staggered times, e.g., sequentially; combination therapy is understood to include all these regimens.

Suitable therapeutic agents for combination therapy with the disclosed compounds include those which modulate a mammalian immune system, antihistamines, and anti-inflammatory agents. These agents may include but are not limited to cyclosporin A (e.g., Sandimmune® or Neoral®, rapamycin, FK-506 (tacrolimus), leflunomide, deoxyspergualin, mycophenolate (e.g., Cellcept®, azathioprine (e.g., Imuran®), daclizumab (e.g., Zenapax®), OKT3 (e.g., Orthocolone®), AtGam, aspirin, acetaminophen, ibuprofen, naproxen, piroxicam, and antiinflammatory steroids (e.g., prednisolone or dexamethasone). These agents may be administered as part of the same or separate dosage forms, via the same or different routes of administration, and on the same or different administration schedules according to standard pharmaceutical practice known to one skilled in the art.

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents. Accordingly, the disclosure is not limited except as by the appended claims.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Any ranges cited herein are inclusive.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

It is understood that one skilled in the art may be able to make these compounds by similar methods or by combining other methods known to one skilled in the art. It is also understood that one skilled in the art would be able to make, in a similar manner as described below, further compounds within the scope of the present disclosure by using appropriate starting materials and modifying the synthetic route as needed. In general, starting materials and reagents can be obtained from commercial vendors or synthesized according to sources known to those skilled in the art or prepared as described herein.

Reagent/reactant names given are as named on the commercial bottle or as generated by IUPAC conventions, ChemDraw 19.0 (CAMBRIDGESOFT®; PerkinElmer). Compounds designated as salts (e.g., hydrochloride, acetate, sulfate) may include more than one molar equivalent of the acid.

### EXEMPLIFICATION

Aspects of the present invention are more fully illustrated by the following examples, which are set forth to illustrate certain aspects of the present invention and are not to be construed as limiting thereof.

### Example 1. Synthesis of 2-(4-(7H-pyrrolo[2,3-d]pyrimidin-4-yl) piperazin-1-yl)-N-(4-(N, N-dimethylsulfamoyl)-3-methoxyphenyl) acetamide (Compound 1)

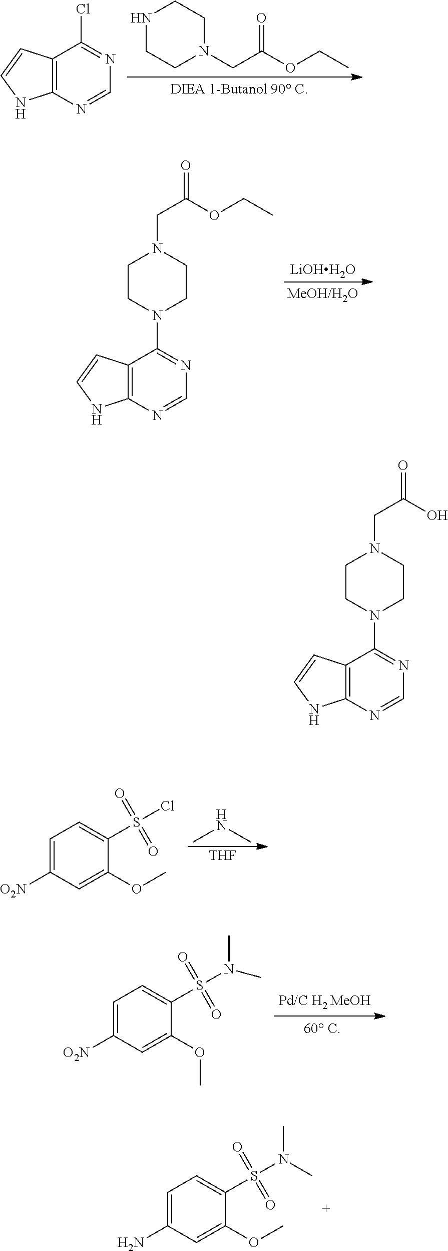

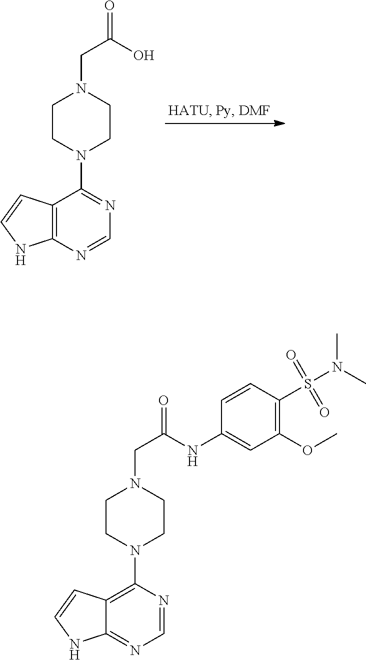 [SMILES: O=C(O)CN1CCN(c2ncnc3[nH]ccc23)CC1 ; COc1cc(NC(=O)CN2CCN(c3ncnc4[nH]ccc34)CC2)ccc1S(=O)(=O)N(C)C]

### Step A. Synthesis of ethyl 2-(4-(7H-pyrrolo[2,3-d]pyrimidin-4-yl) piperazin-1-yl) acetate

To a stirred solution of 4-chloro-7H-pyrrolo[2,3-d]pyrimidine (10.00 g, 65.34 mmol, 1.00 equiv) in 1-Butanol (50 mL) was added ethyl 2-(piperazin-1-yl) acetate (16.86 g, 98.04 mmol, 1.50 equiv) and DIEA (25.29 g, 196.08 mmol, 3.00 equiv) at room temperature. The resulting mixture was stirred at 90° C. for 18 h. The mixture was concentrated, diluted with water and extracted with 1-Butanol. The organic layer was dried over Na₂SO₄ and concentrated under reduced pressure. The residue was purified by silica gel chromatography and eluted with DCM/MeOH=10/1. This resulted in ethyl 2-(4-(7H-pyrrolo[2,3-d]pyrimidin-4-yl) piperazin-1-yl) acetate (18.00 g, 95.0%) as off white. [M+H]⁺=290.

### Step B. Synthesis of 2-(4-(7H-pyrrolo[2,3-d]pyrimidin-4-yl) piperazin-1-yl) acetic acid

To a stirred solution of ethyl 2-(4-(7H-pyrrolo[2,3-d]pyrimidin-4-yl) piperazin-1-yl) acetate (8.00 g, 27.68 mmol, 1.00 equiv) in EtOH/H₂O (20 mL/5 mL) was added LiOH·H₂O (3.49 g, 83.04 mmol, 3.00 equiv) at room temperature. The resulting mixture was stirred at room temperature for 2 h. The mixture was concentrated, diluted with water adjusted with 2N HCl solution to pH=2-3 and then extracted with EtOAc. The combined organic layers were dried over Na₂SO₄, filtered, and concentrated to afford 2-(4-(7H-pyrrolo[2,3-d]pyrimidin-4-yl) piperazin-1-yl) acetic acid (6.36 g, 88.0%) as white solid. [M+H]⁺=263.

### Step C. Synthesis of 2-methoxy-N, N-dimethyl-4-nitrobenzenesulfonamide

A solution of dimethylamine in THF (15 mL, 2M) was added 2-methoxy-4-nitrobenzenesulfonyl chloride (1.00 g, 3.98 mmol, 1.00 equiv) at room temperature. The resulting mixture was stirred at room temperature for 30 min. The mixture was concentrated, diluted with NaHCO₃ solution and then extracted with EtOAc. The combined organic layers were dried over Na₂SO₄, filtered, and concentrated to afford 2-methoxy-N, N-dimethyl-4-nitrobenzenesulfonamide (1.00 g, 100.0%) as yellow solid. [M+H]⁺=261.

### Step D. Synthesis of 4-amino-2-methoxy-N, N-dimethylbenzene sulfonamide

To a stirred solution of 2-methoxy-N, N-dimethyl-4-nitrobenzenesulfonamide (1.00 g, 3.85 mmol, 1.00 equiv) in MeOH (15 mL) was added 10% Pd/C (120.00 mg, 1.13 mmol, 0.29 equiv) at room temperature with H₂. The resulting mixture was stirred at 60° C. for 3 h. The mixture was filtered to afford 4-amino-2-methoxy-N,N-dimethyl benzenesulfonamide (352.00 mg, 40.0%) as yellow solid. [M+H]⁺=231.

### Step E. Synthesis of 2-(4-(7H-pyrrolo[2,3-d]pyrimidin-4-yl) piperazin-1-yl)-N-(4-(N, N-dimethylsulfamoyl)-3-methoxyphenyl) acetamide (Compound 1)

To a solution of 2-(4-(7H-pyrrolo[2,3-d]pyrimidin-4-yl) piperazin-1-yl) acetic acid (170.00 mg, 0.65 mmol, 1.50 equiv) in DMF (10 mL) was added HATU (196.00 mg, 0.52 mmol, 1.20 equiv), Pyridine (102.00 mg, 1.29 mmol, 3.00 equiv) and 4-amino-2-methoxy-N, N-dimethyl benzene sulfonamide (100.00 mg, 0.43 mmol, 1.00 equiv) at room temperature. The resulting mixture was stirred at 40° C. for 18 h. The resulted solution was purified using Prep-HPLC with the following conditions: Column: RP-PREP-5 Xbridge C18 Column, 19*150 mm, 5 μm; Mobile Phase A: Water (0.1% NH₃H₂O), Mobile Phase B: ACN; Flow rate: 15 mL/min; Gradient: 10% B to 55% B in 11 min; wavelength: 214 nm. The title compound was obtained as a white solid (52.00 mg, 25.0%). [M+H]⁺=474. H NMR (400 MHz, CDCl₃) δ 2.71-2.82 (10H, m), 3.19 (2H, s), 3.87 (3H, s), 4.04 (4H, d, J=4.4 Hz), 6.46 (1H, d, J=3.6 Hz), 6.76-6.79 (1H, m), 7.06 (1H, d, J=3.6 Hz), 7.77-7.80 (2H, m), 8.29 (1H, s), 9.25 (1H, s), 10.15 (1H, s).

### Example 2. Synthesis of 2-(4-(7H-pyrrolo[2,3-d]pyrimidin-4-yl)piperazin-1-yl)-N-(4-(N-(tert-butyl)sulfamoyl)phenyl)acetamide (Compound 2)

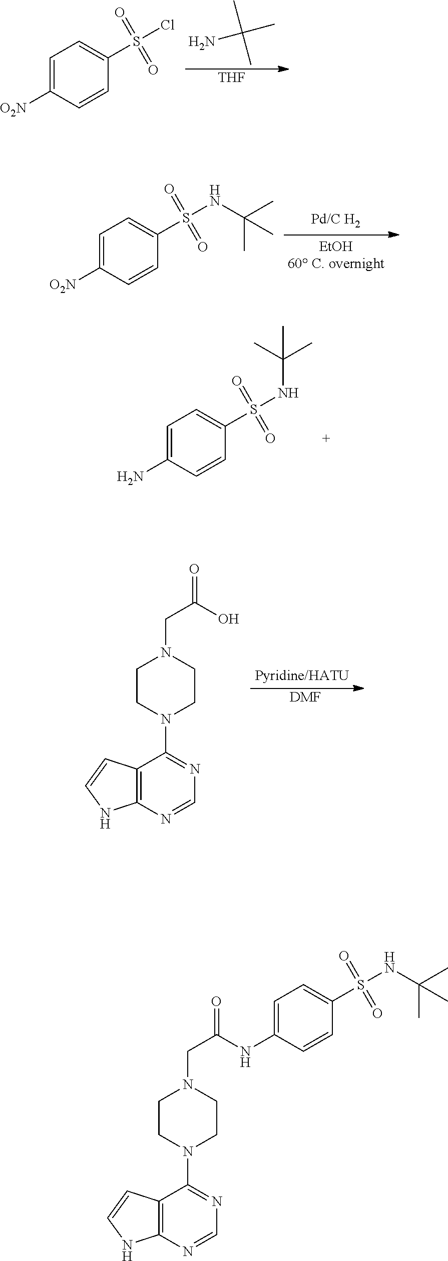

### Step A. Synthesis of N-(tert-butyl)-4-nitrobenzenesulfonamide

To a stirred solution of 4-nitrobenzenesulfonyl chloride (500.00 mg, 2.26 mmol, 1.00 equiv) and 2-methylpropan-2-amine (330.00 mg, 4.52 mmol, 2.00 equiv) in THF (10 mL). The resulting mixture was stirred at 30° C. for 1 h. LCMS showed the reaction was over. The resulting solution was washed with water and extracted with EA (10 mL×3). The solvent was removed to get the desired product. This resulted in N-(tert-butyl)-4-nitrobenzenesulfonamide (532.00 mg, 91.00%) as yellow solid. [M+H]⁺=259.

### Step B. Synthesis of 4-amino-N-(tert-butyl)benzenesulfonamide

To a stirred solution of N-(tert-butyl)-4-nitrobenzenesulfonamide (532.00 mg, 2.06 mmol, 1.00 equiv) in EtOH (20 mL) was added 10% Pd/C (50 mg). The resulting mixture was stirred at 60° C. for overnight under H₂ atmosphere. LCMS showed the reaction was over. The resulting solution was filtered, and the solvent was removed to afford the titled compound (600.00 mg, 100.00%) as yellow oil. [M+H]⁺=229.

### Step C. Synthesis of 2-(4-(7H-pyrrolo[2,3-d]pyrimidin-4-yl)piperazin-1-yl)-N-(4-(N-(tert-butyl)sulfamoyl)phenyl)acetamide (Compound 2)

To a stirred solution of 4-amino-N-(tert-butyl)benzenesulfonamide (100.00 mg, 0.44 mmol, 1.00 equiv) in DMF (5 mL) were added 2-(4-(7H-pyrrolo[2,3-d]pyrimidin-4-yl)piperazin-1-yl)acetic acid (137.00 mg, 0.53 mmol, 1.20 equiv), Pyridine (103.90 mg, 1.32 mmol, 3.00 equiv) and HATU (333.00 mg, 0.88 mmol, 2.00 equiv). The resulting mixture was stirred at 40° C. for 18 h. LCMS showed the desired product was found. The mixture was concentrated in vacuo and the residue was purified by using Prep-HPLC with the following conditions: Column: XBridge C18 SN.271/3611211207 waters, 19*150 mm, 5 μm; Mobile Phase A: Water (0.1% NH4HCO3), Mobile Phase B: ACN; Flow rate: 15 mL/min; Gradient: 20% B to 50% B in 11 min, Wavelength: 214 nm. The title compound was obtained as a white solid (38.00 mg, 18%). [M+H]⁺=472. ¹H NMR (400 MHz, DMSO-d₆): δ 1.08 (9H, s), 2.65-2.67 (4H, m), 3.24 (2H, s), 3.93-3.95 (4H, m), 6.62 (1H, s), 7.18 (1H, s), 7.39 (1H, s), 7.76 (2H, d, J=8.8 Hz), 7.83 (2H, d, J=8.8 Hz), 8.14 (1H, s), 10.13 (1H, s), 11.69 (1H, s).

### Example 3. Synthesis of 2-(4-(7H-pyrrolo[2,3-d]pyrimidin-4-yl)piperazin-1-yl)-N-(4-(N-(tert-butyl)sulfamoyl)phenyl)acetamide (Compound 3)

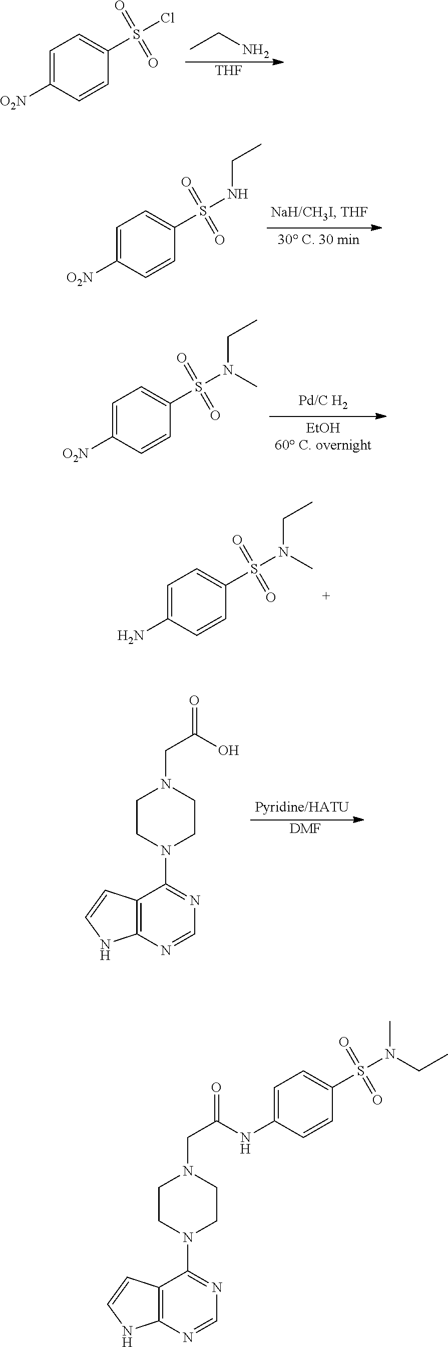

### Step A. Synthesis of N-ethyl-4-nitrobenzenesulfonamide

To a stirred solution of 4-nitrobenzenesulfonyl chloride (1.00 g, 4.52 mmol, 1.00 equiv) in ethanamine in THF (2.0 mol/L, 20 mL, 8.85 equiv). The resulting mixture was stirred at 30° C. for 1 h. LCMS showed the reaction was over. The resulting solution was washed with water and extracted with EA (10 mL×3). The solvent was removed to afford N-ethyl-4-nitrobenzenesulfonamide (1.05 g, 100%) as yellow solid. [M+Na]+=253.

### Step B. Synthesis of N-ethyl-N-methyl-4-nitrobenzenesulfonamide

To a stirred solution of N-ethyl-4-nitrobenzenesulfonamide (500.00 mg, 2.17 mmol, 1.00 equiv) in THF (10 mL) was added 60% NaH (174.00 mg, 2.00 equiv) and Iodomethane (617.00 mg, 4.35 mmol, 2.00 equiv). The resulting mixture was stirred at 30° C. for 30 min. LCMS showed the reaction was over. The resulting solution was washed with water and extracted with EA (10 mL×3). The solvent was removed to afford N-ethyl-N-methyl-4-nitrobenzenesulfonamide (930 mg, 100%) as yellow solid. [M+H]⁺=245.

### Step C. Synthesis of 4-amino-N-ethyl-N-methylbenzenesulfonamide

To a stirred solution of N-ethyl-N-methyl-4-nitrobenzenesulfonamide (930.00 mg, 3.8 mmol, 1.00 equiv) in EtOH (20 mL) was added 10% Pd/C (93 mg). The resulting mixture was stirred at 60° C. for overnight under H₂ atmosphere. LCMS showed the reaction was over. The resulting solution was filtered, and the solvent was removed to afford 4-amino-N-ethyl-N-methylbenzenesulfonamide (500.00 mg, 60.00%) as yellow oil. [M+H]⁺=215.

### Step D. Synthesis of 2-(4-(7H-pyrrolo[2,3-d]pyrimidin-4-yl)piperazin-1-yl)-N-(4-(N-(tert-butyl)sulfamoyl)phenyl)acetamide (Compound 3)

To a stirred solution of 4-amino-N-ethyl-N-methylbenzenesulfonamide (100.00 mg, 0.47 mmol, 1.00 equiv) in DMF (5 mL) were added 2-(4-(7H-pyrrolo[2,3-d]pyrimidin-4-yl)piperazin-1-yl)acetic acid (146.00 mg, 0.56 mmol, 1.20 equiv), Pyridine (111.00 mg, 1.40 mmol, 3.00 equiv) and HATU (533.00 mg, 1.40 mmol, 3.00 equiv). The resulting mixture was stirred at 45° C. for 18 h. LCMS showed the desired product was found. The residue was purified by using Prep-HPLC with the following conditions: Column: XBridge C18 SN.271/3611211207 waters, 19*150 mm, 5 μm; Mobile Phase A: Water (0.1% NH₄HCO₃), Mobile Phase B: ACN; Flow rate: 15 mL/min; Gradient: 25% B to 50% B in 11 min, Wavelength: 214 nm. The title compound (48.00 mg, 22%) was obtained as a white solid. [M+H]⁺=458. ¹H NMR (400 MHz, CDCl₃): δ 1.16 (3H, t, J=7.2 Hz), 2.76 (3H, s), 2.81-2.84 (4H, m), 3.10-3.15 (2H, m), 3.28 (2H, s), 4.10-4.12 (4H, m), 6.54 (1H, s), 7.17 (1H, s), 7.76-7.81 (4H, m), 8.40 (1H, s), 9.39 (1H, s), 10.84 (1H, s).

### Example 4. Synthesis of 2-(4-(7H-pyrrolo[2,3-d]pyrimidin-4-yl) piperazin-1-yl)-N-(4-(azetidin-1-ylsulfonyl) phenyl) acetamide (Compound 4)

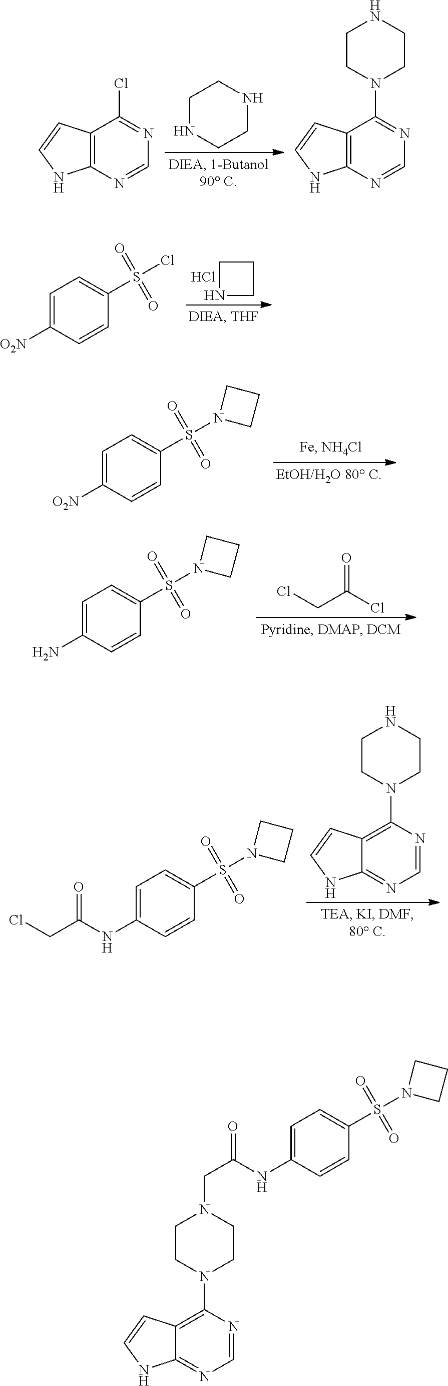

### Step A. Synthesis of 4-(piperazin-1-yl)-7H-pyrrolo[2,3-d]pyrimidine

To a stirred solution of 4-chloro-7H-pyrrolo[2,3-d]pyrimidine (2.56 g, 16.70 mmol, 1.00 equiv) in 1-Butanol (15 mL) was added piperazine (1.44 g, 16.70 mmol, 1.00 equiv) and DIEA (3.23 g, 25.05 mmol, 1.50 equiv) at room temperature. The resulting mixture was stirred at 90° C. for 18 h. The mixture was filtered and concentrated to afford 4-(piperazin-1-yl)-7H-pyrrolo[2,3-d]pyrimidine (3.25 g, 96.0%) as white solid. [M+H]+=204.

### Step B. Synthesis of 1-((4-nitrophenyl) sulfonyl) azetidine

To a stirred solution of 4-nitrobenzenesulfonyl chloride (1.00 g, 4.52 mmol, 1.00 equiv) in THF (15 mL) was added azetidine (0.63 g, 6.79 mmol, 1.50 equiv) and DIEA (1.75 g, 13.56 mmol, 3.00 equiv) at room temperature. The resulting mixture was stirred at room temperature for 2 h. The mixture was diluted with water and then extracted with EtOAc. The combined organic layers were dried over Na₂SO₄, filtered and concentrated to afford 1-((4-nitrophenyl) sulfonyl) azetidine (670 mg, 61.0%) as yellow solid. [M+H]⁺=243.

### Step C. Synthesis of 4-(azetidin-1-ylsulfonyl) aniline

A solution of 1-((4-nitrophenyl) sulfonyl) azetidine (100.00 mg, 0.41 mmol, 1.00 equiv) in EtOH/H₂O (12/3 mL) was added Fe (231.00 mg, 4.13 mmol, 10.00 equiv) and NH₄Cl (110.00 mg, 2.07 mmol, 5.00 equiv) at room temperature. The resulting mixture was stirred at 80° C. for 1 h. The mixture was filtered and concentrated, diluted with NaHCO₃ solution and then extracted with EtOAc. The combined organic layers were dried over Na₂SO₄, filtered, and concentrated to afford 4-(azetidin-1-ylsulfonyl) aniline (60.00 mg, 68.0%) as yellow solid. [M+H]⁺=213.

### Step D. Synthesis of N-(4-(azetidin-1-ylsulfonyl) phenyl)-2-chloroacetamide

To a stirred solution of 4-(azetidin-1-ylsulfonyl) aniline (60.00 mg, 0.28 mmol, 1.00 equiv) in DCM (10 mL) was added pyridine (44.00 mg, 0.56 mmol, 2.00 equiv), DMAP (3.00 mg, 0.03 mmol, 0.10 equiv) was added 2-chloroacetyl chloride (32.00 mg, 0.28 mmol, 1.00 equiv) at room temperature. The resulting mixture was stirred at room temperature for 15 min. The mixture was diluted with H₂O and t extracted with DCM. The combined organic layers were dried over Na₂SO₄, filtered, and concentrated to afford N-(4-(azetidin-1-ylsulfonyl) phenyl)-2-chloroacetamide (81.00 mg, 100%) as yellow solid. [M+H]⁺=289.

### Step E. Synthesis of 2-(4-(7H-pyrrolo[2,3-d]pyrimidin-4-yl) piperazin-1-yl)-N-(4-(azetidin-1-ylsulfonyl) phenyl) acetamide (Compound 4)

To a solution of N-(4-(azetidin-1-ylsulfonyl) phenyl)-2-chloroacetamide (81.00 mg, 0.28 mmol, 1.00 equiv) in DMF (10 mL) was added 4-(piperazin-1-yl)-7H-pyrrolo[2,3-d]pyrimidine (57.00 mg, 0.28 mmol, 1.00 equiv), TEA (141.00 mg, 1.40 mmol, 5.00 equiv) and KI (23.00 mg, 0.14 mmol, 0.50 equiv) at room temperature. The resulting mixture was stirred at 80° C. for 1 h. The resulted solution was purified using Prep-HPLC with the following conditions: Column: RP-PREP-5 Xbridge C18 Column, 19*150 mm, 5 μm; Mobile Phase A: Water (0.1% NH₄HCO₃), Mobile Phase B: ACN; Flow rate: 15 mL/min; Gradient: 35% B to 65% B in 10 min; Wavelength: 214 nm. The title compound (46.00 mg, 36.0%) was obtained as a white solid. [M+H]⁺=456. ¹H NMR (400 MHz, CDCl₃): δ 2.06-2.14 (2H, m), 2.84 (4H, t, J=5.2 Hz), 3.30 (2H, s), 3.78-3.82 (4H, m), 4.10-4.13 (4H, m), 6.55 (1H, d, J=4.0 Hz), 7.15 (1H, d, J=3.6 Hz), 7.88-7.83 (4H, m), 8.39 (1H, s), 9.44 (1H, s), 10.23 (1H, s).

### Example 5. Synthesis of 2-(4-(7H-pyrrolo[2,3-d]pyrimidin-4-yl)piperazin-1-yl)-N-(4-(N,N-dimethylsulfamoyl)-3-fluorophenyl)acetamide (Compound 5)

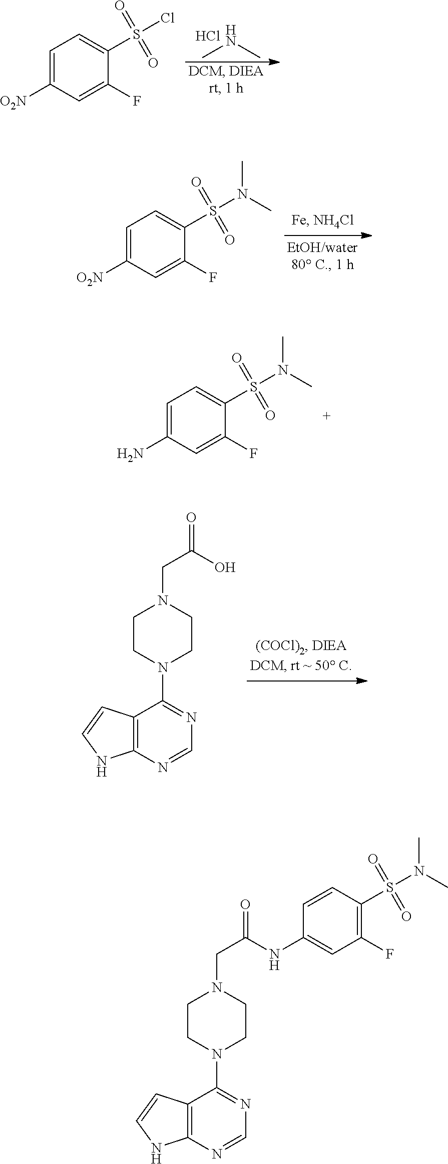

### Step A. Synthesis of 2-fluoro-N,N-dimethyl-4-nitrobenzenesulfonamide

To a solution of 2-fluoro-4-nitrobenzenesulfonyl chloride (300.00 mg, 1.25 mmol, 1.00 equiv) and Dimethylamine hydrochloride (112.75 mg, 1.37 mmol, 1.10 equiv) in DCM (10 mL) was added DIEA (322.00 mg, 2.50 mmol, 2.00 equiv). The mixture was stirred at room temperature for 1 h. The LCMS showed the reaction was finished. The residue was extracted with DCM and washed with brine. The organic layer was dried over Na₂SO₄ and concentrated to afford 2-fluoro-N,N-dimethyl-4-nitrobenzenesulfonamide (280 mg, 90.3%) as off-white solid. [M+H]⁺=249.

### Step B. Synthesis of 4-amino-2-fluoro-N,N-dimethylbenzenesulfonamide

To a solution of 2-fluoro-N,N-dimethyl-4-nitrobenzenesulfonamide (130.00 mg, 0.52 mmol, 1.00 equiv) in EtOH/water (10 mL/2 mL) was added Fe (293.00 mg, 5.20 mmol, 10.00 equiv) and NH₄Cl (140.00 mg, 2.60 mmol, 5.00 equiv). The mixture was stirred at 80° C. for 1 h. The LCMS showed the reaction was finished. The mixture was filtered and the filtrate was extracted with EtOAc and washed with water. The organic layer was dried over Na₂SO₄ and concentrated to afford 4-amino-2-fluoro-N,N-dimethylbenzenesulfonamide (161.00 mg, >100%) as gray solid. [M+H]=219.

### Step C. Synthesis of 2-(4-(7H-pyrrolo[2,3-d]pyrimidin-4-yl)piperazin-1-yl)-N-(4-(N,N-dimethylsulfamoyl)-3-fluorophenyl)acetamide (Compound 5)

To a solution of 2-(4-(7H-pyrrolo[2,3-d]pyrimidin-4-yl)piperazin-1-yl)acetic acid (90.00 mg, 0.34 mmol, 1.00 equiv) in DCM (10 mL) was added (COCl)₂ (131.00 mg, 1.03 mmol, 3.00 equiv) and one drop of DMF. The mixture was stirred at room temperature for 2 h, concentrated to get the residue. To a solution of 4-amino-2-fluoro-N,N-dimethylbenzenesulfonamide (90.00 mg, 0.41 mmol, 1.20 equiv) and DIEA (88.70 mg, 0.69 mmol, 2.00 equiv) in DCM (10 mL) was added the solution of above residue in DCM. The mixture was stirred at 80° C. for 3 h. The LCMS showed the reaction was finished. The mixture was concentrated. The residue was purified by using Prep-HPLC with the following conditions: Column: XBridge Prep C18 Column, 19*150 mm, 5 μm; Mobile Phase A: Water (0.1% NH₄HCO₃), Mobile Phase B: ACN; Flow rate: 15 mL/min; Gradient: 25% B to 40% B in 12 min, Wavelength: 214 nm. The title compound (6.00 mg, 3.1%) was obtained as an off-white solid. [M+H]+=462. ¹H NMR (400 MHz, DMSO-d₆): δ 2.64-2.69 (10H, m), 3.27 (2H, s), 3.94 (4H, t, J=4.8 Hz), 6.62 (1H, d, J=3.2 Hz), 7.19 (1H, d, J=3.6 Hz), 7.62-7.65 (1H, m), 7.73 (1H, t, J=8.8 Hz), 7.88-7.92 (1H, m), 8.15 (1H, s), 10.42 (1H, s), 11.70 (1 H, s).

### Example 6. Synthesis of 2-(4-(7H-pyrrolo[2,3-d]pyrimidin-4-yl)piperazin-1-yl)-N-(3-chloro-4-(N,N-dimethylsulfamoyl)phenyl)acetamide (Compound 6)

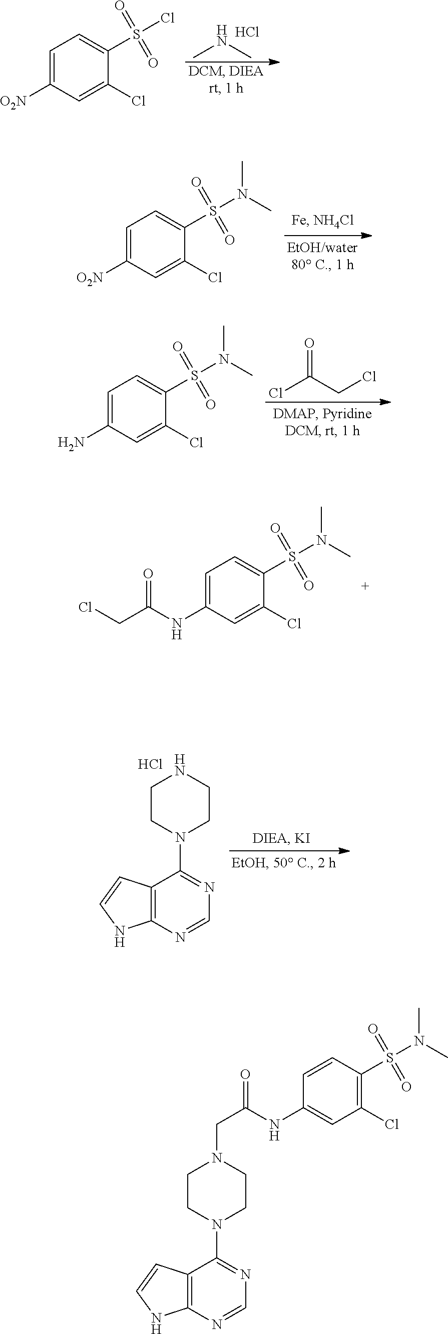

### Step A. Synthesis of 2-chloro-N,N-dimethyl-4-nitrobenzenesulfonamide

To a solution of 2-chloro-4-nitrobenzenesulfonyl chloride (512.00 mg, 2.00 mmol, 1.00 equiv) and Dimethylamine hydrochloride (200.00 mg, 2.40 mmol, 1.20 equiv) in DCM (15 mL) was added DIEA (516.00 mg, 4.00 mmol, 2.00 equiv). The mixture was stirred at room temperature for 1 h. The LCMS showed the reaction was finished. The residue was extracted with DCM and washed with brine. The organic layer was dried over Na₂SO₄ and concentrated to afford 2-chloro-N,N-dimethyl-4-nitrobenzenesulfonamide (518.00 mg, 98.1%) as off-white solid. [M+H]⁺=265.

### Step B. Synthesis of 4-amino-2-chloro-N,N-dimethylbenzenesulfonamide

To a solution of 2-chloro-N,N-dimethyl-4-nitrobenzenesulfonamide (518.00 mg, 1.96 mmol, 1.00 equiv) in EtOH/water (10 mL/3 mL) was added Fe (1.10 g, 19.60 mmol, 10.00 equiv) and NH₄Cl (520.00 mg, 9.80 mmol, 5.00 equiv). The mixture was stirred at 80° C. for 1 h. The LCMS showed the reaction was finished. The mixture was filtered and the filtrate was extracted with DCM and washed with water. The organic layer was dried over Na₂SO₄ and concentrated to afford 4-amino-2-chloro-N,N-dimethylbenzenesulfonamide (440.00 mg, 96.1%) as gray solid. [M+H]⁺=235.

### Step C. Synthesis of 2-chloro-N-(3-chloro-4-(N,N-dimethylsulfamoyl)phenyl)acetamide

To a solution of 4-amino-2-chloro-NN-dimethylbenzenesulfonamide (200.00 mg, 0.85 mmol, 1.00 equiv), pyridine (135.00 mg, 1.71 mmol, 2.00 equiv) and DMAP (10.00 mg, 0.08 mmol, 0.10 equiv) in DCM (10 mL) was added 2-chloroacetyl chloride (97.00 mg, 0.85 mmol, 1.00 equiv). The mixture was stirred at room temperature for 1 h. The LCMS showed the reaction was finished. The residue was extracted with DCM and washed with brine. The organic layer was dried over Na₂SO₄ and concentrated to afford 2-chloro-N-(3-chloro-4-(N,N-dimethylsulfamoyl)phenyl)acetamide (304 mg, >100%) as brown solid. [M+H]⁺=311.

### Step C. Synthesis of 2-(4-(7H-pyrrolo[2,3-d]pyrimidin-4-yl)piperazin-1-yl)-N-(3-chloro-4-(N,N-dimethylsulfamoyl)phenyl)acetamide (Compound 6)

To a solution of 4-(piperazin-1-yl)-7H-pyrrolo[2,3-d]pyrimidine (HCl salt; 80.00 mg, 0.39 mmol, 1.00 equiv) and 2-chloro-N-(3-chloro-4-(N,N-dimethylsulfamoyl)phenyl)acetamide (121 mg, 0.39 mmol, 1.00 equiv) in EtOH (10 mL) was added TEA (196.00 mg, 1.96 mmol, 5.00 equiv) and KI (33.00 mg, 0.20 mmol, 0.50 equiv). The mixture was stirred at 80° C. for 2 h. The LCMS showed the reaction was finished. The mixture was concentrated. The residue was purified by using Prep-HPLC with the following conditions: Column: XBridge Prep C18 Column, 19*150 mm, 5 μm; Mobile Phase A: Water (0.1% NH₃ H₂O), Mobile Phase B: ACN; Flow rate: 15 mL/min; Gradient: 10% B to 55% B in 10 min, Wavelength: 214 nm. The title compound (43 mg, 23%) was obtained as a white solid. [M+H]⁺=478. ¹H NMR (400 MHz, DMSO-d₆): δ 2.65 (4H, t, J=4.4 Hz), 2.77 (6H, s), 3.26 (2H, s), 3.94 (4H, t, J=4.4 Hz), 6.61 (1H, d, J=3.2 Hz), 7.18 (1H, d, J=3.2 Hz), 7.80-7.82 (1H, m), 7.90 (1H, d, J=8.8 Hz), 8.09 (1H, d, J=2.4 Hz), 8.15 (1H, s), 10.34 (1H, s), 11.70 (1H, s).

### Example 7. Synthesis of 2-(4-(7H-pyrrolo[2,3-d]pyrimidin-4-yl)piperazin-1-yl)-N-(4-(N-cyclopropylsulfamoyl)phenyl)acetamide (Compound 7)

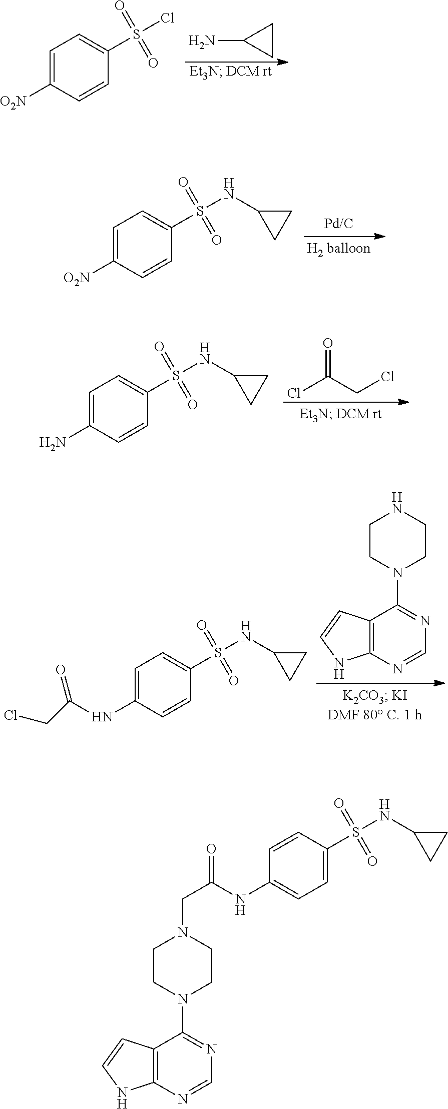

### Step A. Synthesis of N-cyclopropyl-4-nitrobenzenesulfonamide

To a solution of cyclopropanamine (130 mg, 2.20 mmol, 1.00 equiv) in DCM/Et₃N (10 ml/ml) was added 4-nitrobenzenesulfonyl chloride (500 mg, 2.20 mmol, 1.00 equiv). The resulting mixture was stirred at room temperature for 1 h and monitored by TLC. The mixture was extracted with ethyl acetate and water, the combined organic layers were evaporated under reduced pressure to afford crude N-cyclopropyl-4-nitrobenzenesulfonamide (530.00 mg, 96.36%) as yellow oil. [M+H]⁺=N/A

### Step B. Synthesis of 4-amino-N-cyclopropylbenzenesulfonamide

A flask was charged with N-cyclopropyl-4-nitrobenzenesulfonamide (530 mg, 2.19 mmol, 1.00 equiv) in MeOH (30 mL) under H₂ balloon. The reaction was stirred at room temperature for 16 h and monitored by LC-MS. After the starting material consumed, the mixture was filtered and evaporated under reduced pressure to afford the crude titled product (500.00 mg, >100%) as grey solid. [M+H]=213

### Step C. Synthesis of 2-chloro-N-(4-(N-cyclopropylsulfamoyl)phenyl)acetamide

To a solution of (50 mg, 0.23 mmol 1.00 equiv) in DCM/Et₃N (5 ml/0.2 ml) was added 2-chloroacetyl chloride (30 mg, 0.23 mmol, 1.00 equiv). The resulting mixture was stirred at room temperature for 1 h and monitored by TLC. The mixture was extracted with ethyl acetate and water, the combined organic layers were evaporated under reduced pressure to afford crude 2-chloro-N-(4-(N-cyclopropylsulfamoyl)phenyl)acetamide (80 mg, >100.00%) as yellow oil. [M+H]⁺=N/A

### Step D. Synthesis of 2-(4-(7H-pyrrolo[2,3-d]pyrimidin-4-yl)piperazin-1-yl)-N-(4-(N-cyclopropylsulfamoyl)phenyl)acetamide (Compound 7)

To a solution of 4-(piperazin-1-yl)-7H-pyrrolo[2,3-d]pyrimidine (80.00 mg, 0.27 mmol, 1.10 equiv) and 2-chloro-N-(4-(N-cyclopropylsulfamoyl)phenyl)acetamide (50.00 mg, 0.27 mmol, 1.00 equiv) in DMF (8 mL) was added K₂CO₃ (100.00 mg, 0.81 mmol, 3.00 equiv) and KI (20.00 mg, 0.13 mmol, 0.50 equiv). The mixture was stirred at 80° C. for 1 h. The LCMS showed the reaction was finished. The mixture was concentrated. The residue was purified by using Prep-HPLC with the following conditions: Column: XBridge C18 Column, 19*150 mm, 5 μm; Mobile Phase A: Water (0.1% NH4HCO3), Mobile Phase B: ACN; Gradient: 15% B to 60% B in 10 min; Flow rate: 15 mL/min; Wavelength: 214 nm. The title compound (20 mg, 15%) was obtained as a white solid. [M+H]=456. ¹H NMR (400 MHz, DMSO-d₆): δ 0.34-0.37 (2H, m), 0.44-0.49 (2H, m), 2.07-2.11 (1H, m), 2.65-2.67 (4H, m), 3.26 (2H, s), 3.93-3.95 (4H, m), 6.61-6.62 (1H, m), 7.18-7.19 (1H, m), 7.74-7.76 (3H, m), 7.88 (2H, d, J=8.8 Hz), 8.15 (1H, s), 10.18 (1H, s), δ 11.69 (1H, s).

### Example 8. Synthesis of 2-(4-(7H-pyrrolo[2,3-d]pyrimidin-4-yl)piperazin-1-yl)-N-(4-(N-methylsulfamoyl)phenyl)acetamide (Compound 8)

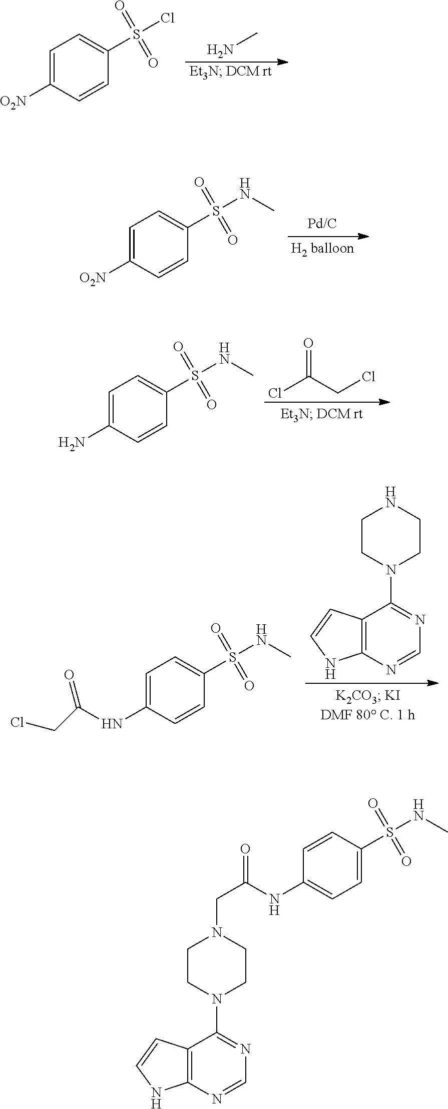

### Step A. Synthesis of N-methyl-4-nitrobenzenesulfonamide

To a solution of methanamine (500 mg, 1.30 mmol, 1.00 equiv) in DCM/Et₃N (10 ml/1 ml) was added 4-nitrobenzenesulfonyl chloride (300 mg, 1.30 mmol, 1.00 equiv). The resulting mixture was stirred at room temperature for 1 h and monitored by TLC. The mixture was extracted with ethyl acetate and water, the combined organic layers were evaporated under reduced pressure to afford the crude N-cyclopropyl-4-nitrobenzenesulfonamide (280.00 mg, 96.55%) as yellow oil. [M+H]⁺=N/A.

### Step B. Synthesis of 4-amino-N-methylbenzenesulfonamide

A flask was charged with N-cyclopropyl-4-nitrobenzenesulfonamide (280 mg, 1.30 mmol, 1.00 equiv) and 10% Pd/C (60.00 mg) in MeOH (30 mL) under H₂ balloon. The reaction was stirred at room temperature for 16 h and monitored by LC-MS. After the starting material consumed, the mixture was filtered and evaporated under reduced pressure to afford crude 4-amino-N-methylbenzenesulfonamide (240 mg, >100.00%) as yellow solid. [M+H]⁺=187.

### Step C. Synthesis of 2-chloro-N-(4-(N-methylsulfamoyl)phenyl)acetamide

To a solution of 4-amino-N-methylbenzenesulfonamide (100.00 mg, 0.53 mmol 1.00 equiv) in DCM/Et₃N (8 ml/0.2 ml) was added 2-chloroacetyl chloride (60.00 mg, 0.53 mmol, 1.00 equiv). The resulting mixture was stirred at room temperature for 1 h and monitored by TLC. The mixture was extracted with ethyl acetate and water, the combined organic layers were evaporated under reduced pressure to afford crude 2-chloro-N-(4-(N-methylsulfamoyl)phenyl)acetamide (60 mg, 42.80%) as yellow oil. [M+H]⁺=263.

### Step D. Synthesis of 2-(4-(7H-pyrrolo[2,3-d]pyrimidin-4-yl)piperazin-1-yl)-N-(4-(N-methylsulfamoyl)phenyl)acetamide (Compound 8)

To a solution of 4-(piperazin-1-yl)-7H-pyrrolo[2,3-d]pyrimidine (47 mg 0.22 mmol, 1.00 equiv) and 2-chloro-N-(4-(N-cyclopropylsulfamoyl)phenyl)acetamide (60 mg, 0.22 mmol, 1.00 equiv) in DMF (8 mL) was added K₂CO₃ (100 mg, 0.66 mol, 3.00 equiv) and KI (20 mg, 0.13 mmol, 0.5 equiv). The mixture was stirred at 80° C. for 1 h. The LCMS showed the reaction was complete. The mixture was concentrated. The residue was purified by using Prep-HPLC with the following conditions: Column: XBridge C18 Column, 19*150 mm, 5 μm; Mobile Phase A: Water (0.1% NH₄HCO₃), Mobile Phase B: ACN; Gradient: 20% B to 65% B in 10 min; Flow rate: 15 mL/min; Wavelength: 214 nm. The title compound (20 mg, 15%) was obtained as a white solid. [M+H]=430. ¹H NMR (400 MHz, DMSO-d₆): 2.39 (3H, d, J=5.2 Hz), 2.64-2.66 (4H, m), 3.25 (2H, s), 3.92-3.95 (4H, s), 6.61-6.62 (1H, m), 7.18-7.19 (1H, m), 7.31 (1H, d, J=5.2 Hz), 7.71-7.73 (2H, m), 7.86-7.88 (2H, m), 8.17 (1H, s), 10.18 (1H, s), δ 11.70 (1H, s).

### Example 9. Synthesis of 2-(4-(7H-pyrrolo[2,3-d]pyrimidin-4-yl)piperazin-1-yl)-N-(3-fluoro-4-(N-methylsulfamoyl)phenyl)acetamide (Compound 9)

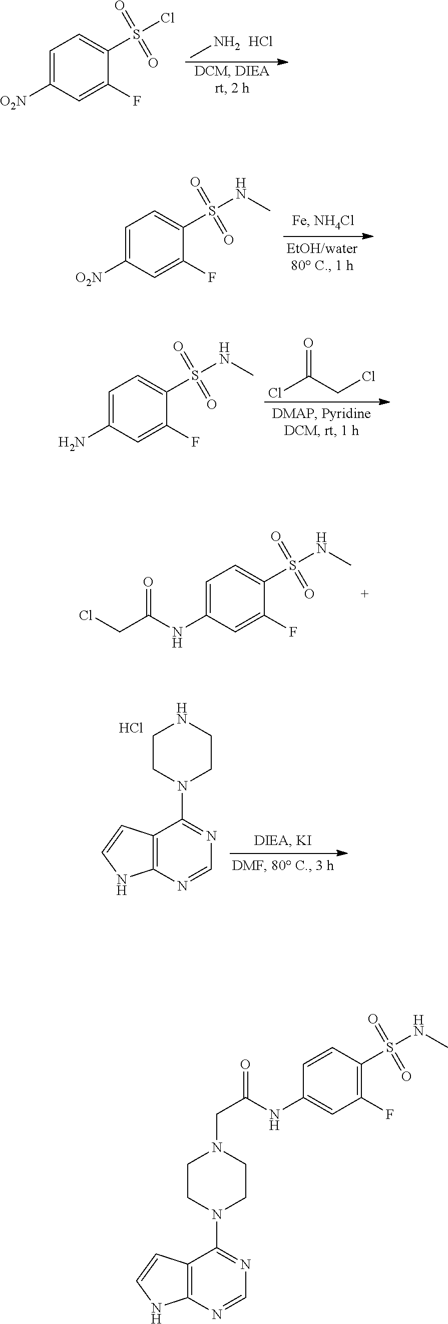

### Step A. Synthesis of 2-fluoro-N-methyl-4-nitrobenzenesulfonamide

To a solution of 2-fluoro-4-nitrobenzenesulfonyl chloride (400.00 mg, 1.67 mmol, 1.00 equiv) and Methylamine hydrochloride (134.00 mg, 2.0 mmol, 1.20 equiv) in DCM (10 mL) was added DIEA (431.00 mg, 3.34 mmol, 2.00 equiv). The mixture was stirred at room temperature for 2 h. The LCMS showed the reaction was finished. The residue was extracted with DCM and washed with brine. The organic layer was dried over Na₂SO₄ and concentrated. The residue was purified by column eluted with PE/EA=4/1 to afford 2-fluoro-N-methyl-4-nitrobenzenesulfonamide (103 mg, 26.4%) as yellow solid. [M+H]⁺=235.

### Step B. Synthesis of 4-amino-2-fluoro-N-methylbenzenesulfonamide

To a solution of 2-fluoro-N-methyl-4-nitrobenzenesulfonamide (101.00 mg, 0.43 mmol, 1.00 equiv) in EtOH/water (5 mL/1 mL) was added Fe powder (241 mg, 4.30 mmol, 10.00 equiv) and NH₄Cl (115.00 mg, 2.14 mmol, 5.00 equiv). The mixture was stirred at 80° C. for 1 h. The LCMS showed the reaction was finished. The mixture was filtered, and the filtrate was extracted with DCM and washed with water. The organic layer was dried over Na₂SO₄ and concentrated to afford 4-amino-2-fluoro-N-methylbenzenesulfonamide (81 mg, 93.1%) as yellow solid. [M+H]⁺=205.

### Step C. Synthesis of 2-chloro-N-(3-fluoro-4-(N-methylsulfamoyl)phenyl)acetamide

To a solution of 4-amino-2-fluoro-N-methylbenzenesulfonamide (80 mg, 0.39 mmol, 1.00 equiv), pyridine (53.70 mg, 0.68 mmol, 2.00 equiv) and DMAP (4.76 mg, 0.04 mmol, 0.10 equiv) in DCM (10 mL) was added 2-chloroacetyl chloride (44 mg, 0.39 mmol, 1.00 equiv). The mixture was stirred at room temperature for 1 h. The LCMS showed the reaction was finished. The residue was extracted with DCM and washed with brine. The organic layer was dried over Na₂SO₄ and concentrated to afford 2-chloro-N-(3-fluoro-4-(N-methylsulfamoyl)phenyl)acetamide (135 mg, >100%) as yellow solid. [M+H]⁺=281.

### Step D. Synthesis of 2-(4-(7H-pyrrolo[2,3-d]pyrimidin-4-yl)piperazin-1-yl)-N-(3-fluoro-4-(N-methylsulfamoyl)phenyl)acetamide (Compound 9)

To a solution of 4-(piperazin-1-yl)-7H-pyrrolo[2,3-d]pyrimidine (HCl salt) (127.00 mg, 0.30 mmol, 1.10 equiv) and 2-chloro-N-(3-fluoro-4-(N-methylsulfamoyl)phenyl)acetamide (135.00 mg, 0.48 mmol, 1.00 equiv) in DMF (10 mL) was added DIEA (186.00 mg, 1.44 mmol, 3.00 equiv) and KI (40.00 mg, 0.24 mmol, 0.50 equiv).

The mixture was stirred at 80° C. for 3 h. The LCMS showed the reaction was finished. The mixture was concentrated. The residue was purified by using Prep-HPLC with the following conditions: Column: XBridge Prep C18 Column, 19*150 mm, 5 μm; Mobile Phase A: Water (0.1% NH₄HCO₃), Mobile Phase B: ACN; Flow rate: 15 mL/min; Gradient: 10% B to 65% B in 10 min, Wavelength: 214 nm. The title compound (62.00 mg, 29%) was obtained as a white solid. [M+H]=448. ¹H NMR (400 MHz, DMSO-d₆): δ 2.46 (3H, d, J=4.4 Hz), 2.65 (4H, t, J=4.8 Hz), 3.27 (2H, s), 3.94 (4H, t, J=4.4 Hz), 6.61-6.62 (1H, m), 7.18-7.20 (1H, m), 7.57-7.60 (2H, m), 7.69-7.75 (1H, m), 7.84-7.88 (1H, m), 8.15 (1H, s), 10.35 (1H, s), 11.71 (1H, s).

### Example 10. Synthesis of 2-(4-(7H-pyrrolo[2,3-d]pyrimidin-4-yl)piperazin-1-yl)-N-(4-(N-(2-methoxyethyl)-N-methylsulfamoyl)phenyl)acetamide (Compound 10)

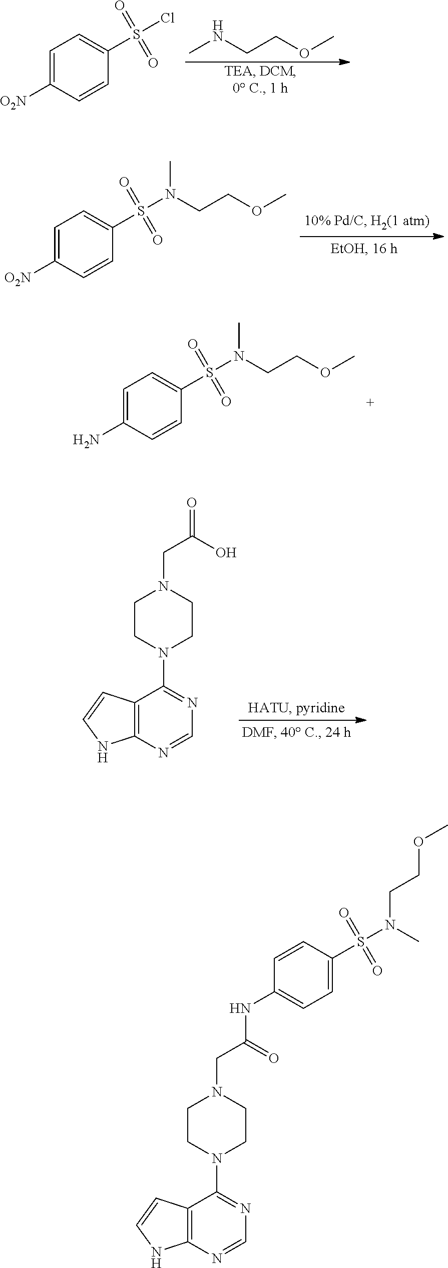

### Step A. Synthesis of N-(2-methoxyethyl)-N-methyl-4-nitrobenzenesulfonamide

To a solution of 4-nitrobenzenesulfonyl chloride (2.00 g, 9.05 mmol, 1.00 equiv) in DCM (30 mL) were added TEA (1.83 g, 18.10 mmol, 2.00 equiv) and 2-methoxy-N-methylethan-1-amine (1.21 g, 13.58 mmol, 1.50 equiv) at 0° C. The resulting mixture was stirred at 0° C. for 1 h. The residue was purified by silica gel column chromatography (PE/EtOAc═O˜30%) to afford N-(2-methoxyethyl)-N-methyl-4-nitrobenzenesulfonamide (2.40 g, 96.8%) as a yellow solid.

### Step B. Synthesis of 4-amino-N-(2-methoxyethyl)-N-methylbenzenesulfonamide

To a solution of N-(2-methoxyethyl)-N-methyl-4-nitrobenzenesulfonamide (500 mg, 1.82 mmol, 1.00 equiv) in EtOH (15 mL) was added Pd/C (100 mg) at room temperature. The resulting mixture was stirred at room temperature for 1 h under H₂ (1 atm). The mixture was filtered and dried to afford 4-amino-N-(2-methoxyethyl)-N-methylbenzenesulfonamide (430 mg, 96.6%) as a yellow solid, which was used directly in the next step without further purification. [M+H]⁺=245.

### Step C. Synthesis of 2-(4-(7H-pyrrolo[2,3-d]pyrimidin-4-yl)piperazin-1-yl)-N-(4-(N-(2-methoxyethyl)-N-methylsulfamoyl)phenyl)acetamide (Compound 10)

To a stirred solution of 2-(4-(7H-pyrrolo[2,3-d]pyrimidin-4-yl)piperazin-1-yl)acetic acid (100 mg, 0.38 mmol, 1.00 equiv) and HATU (216.60 mg, 0.57 mmol, 1.50 equiv) in DMF (15 mL) were added pyridine (120.10 mg, 1.52 mmol, 4.00 equiv) and 4-amino-N-(2-methoxyethyl)-N-methylbenzenesulfonamide (92.72 mg, 0.38 mmol, 1.00 equiv) at room temperature. The resulting mixture was stirred at 40° C. for 1 h. The DMF was removed, extracted with EA, dried over Na₂SO₄, filtered. And the resulted solution was purified using Prep-HPLC with the following conditions: Column: RP-PREP-1 XBridge C18 Column, 90*150 mm, 5 μm; Mobile Phase A: Water (0.1% NH₄HCO₃), Mobile Phase B: ACN; Flow rate: 15 mL/min; Gradient: 20% B to 40% B in 10 min; Wavelength: 214 nm. The title compound (35 mg, 18.8%) was obtained as a white solid. [M+H]=488, H NMR (400 MHz, DMSO-d₆): δ 2.50-2.69 (7H, m), 3.10 (2H, t, J=5.6 Hz), 3.23-3.25 (5H, m), 3.44 (2H, t, J=5.6 Hz), 3.94 (4H, t, J=4.4 Hz), 6.62 (1H, d, J=1.6 Hz), 7.18 (1H, t, J=2.8 Hz), 7.73 (2H, d, J=8.8 Hz), 7.90 (2H, d, J=8.4 Hz), 8.14 (1H, s), 10.22 (1H, s), 11.69 (1H, s).

### Example 11. Synthesis of 2-(4-(7H-pyrrolo[2,3-d]pyrimidin-4-yl)piperazin-1-yl)-N-(4-(N-butylsulfamoyl)phenyl)acetamide (Compound 11)

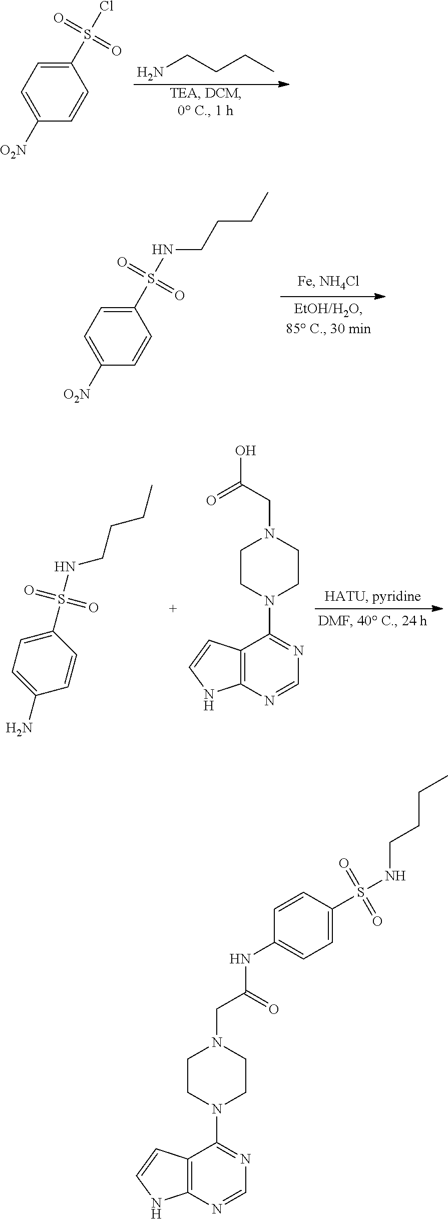

### Step A. Synthesis of N-butyl-4-nitrobenzenesulfonamide

To a solution of 4-nitrobenzenesulfonyl chloride (2.00 g, 9.05 mmol, 1.00 equiv) in DCM (30 mL) were added TEA (1.83 g, 18.10 mmol, 2.00 equiv) and butan-1-amine (990.98 mg, 13.58 mmol, 1.50 equiv) at 0° C. The resulting mixture was stirred at 0° C. for 1 h. The residue was purified by silica gel column chromatography (PE/EtOAc═O˜30%) to afford N-butyl-4-nitrobenzenesulfonamide (900 mg, 38.6%) as a yellow solid.

### Step B. Synthesis of 4-amino-N-butylbenzenesulfonamide

To a solution of N-butyl-4-nitrobenzenesulfonamide (900 mg, 3.49 mmol, 1.00 equiv) in EtOH (30.00 mL) and H₂O (10.00 mL) were added Fe powder (977 mg, 17.45 mmol, 5.00 equiv) and NH₄Cl (1.88 g, 34.90 mmol, 10.00 equiv) at room temperature. The resulting mixture was stirred at 80° C. for 30 min. The mixture was cooled to room temperature, filtered, the solvent was removed in vacuo, then extracted with EA, dried over Na₂SO₄, filtered, and dried to afford 4-amino-N-butylbenzenesulfonamide (780.00 mg, 97.7%) as a yellow solid, which was used directly in the next step without further purification. [M+H]⁺=229.

### Step C. Synthesis of 2-(4-(7H-pyrrolo[2,3-d]pyrimidin-4-yl)piperazin-1-yl)-N-(4-(N-butylsulfamoyl)phenyl)acetamide (Compound 11)

To a stirred solution of 2-(4-(7H-pyrrolo[2,3-d]pyrimidin-4-yl)piperazin-1-yl)acetic acid (100 mg, 0.38 mmol, 1.00 equiv) and HATU (216.60 mg, 0.57 mmol, 1.50 equiv) in DMF (15 mL) were added pyridine (120.10 mg, 1.52 mmol, 4.00 equiv) and 4-amino-N-butylbenzenesulfonamide (86.60 mg, 0.38 mmol, 1.00 equiv) at room temperature. The resulting mixture was stirred at 40° C. for 1 h. The DMF was removed, extracted with EA, dried over Na₂SO₄, filtered. And the resulted solution was purified using Prep-HPLC with the following conditions: Column: RP-PREP-1 XBridge C18 Column, 90*150 mm, 5 μm; Mobile Phase A: Water(0.1% NH4HCO3), Mobile Phase B: ACN; Flow rate: 15 mL/min; Gradient: 30% B to 42% B in 12 min; Wavelength: 214 nm; The title compound (35 mg, 19.4%) was obtained as a white solid. [M+H]=472. ¹H NMR (400 MHz, DMSO-d₆): δ 0.79 (3H, t, J=7.2 Hz), 1.20-1.25 (2H, m), 1.30-1.35 (2H, m), 2.65-2.72 (6H, m), 3.25 (2H, s), 3.93 (4H, d, J=5.4 Hz), 6.62 (1H, t, J=1.6 Hz), 7.18 (1H, t, J=2.4 Hz), 7.42 (1H, t, J=5.6 Hz), 7.73 (2H, d, J=8.8 Hz), 7.86 (2H, d, J=8.8 Hz), 8.17 (1H, s), 10.16 (1H, s), 11.69 (1H, s).

### Example 14. Synthesis of 2-(4-(7H-pyrrolo[2,3-d]pyrimidin-4-yl)piperazin-1-yl)-N-(4-(N-propylsulfamoyl)phenyl)acetamide (Compound 12)

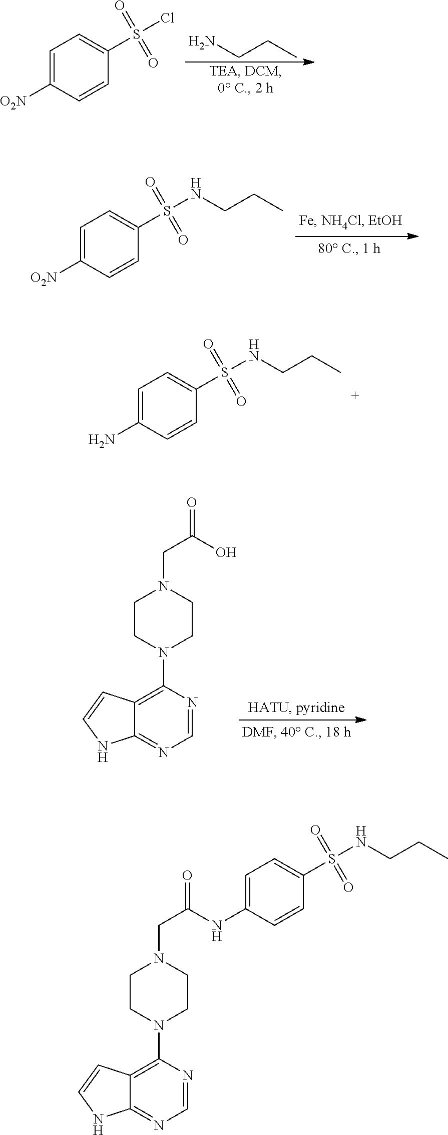

### Step A. Synthesis of 4-nitro-N-propylbenzenesulfonamide

To a solution of 4-nitrobenzenesulfonyl chloride (2.20 g, 10.00 mmol, 1.00 equiv) in DCM (50 mL) were added TEA (3.03 g, 30.00 mmol, 3.00 equiv) and propan-1-aminee (0.88 g, 15.00 mmol, 1.50 equiv) at 0° C. The resulting mixture was stirred at 0° C. for 2 h. The residue was purified by silica gel column chromatography (PE/EtOAc═O˜30%) to afford 4-nitro-N-propylbenzenesulfonamide (2.20 g, 90.5%) as a yellow solid.

### Step B. Synthesis of 4-amino-N-propylbenzenesulfonamide

To a solution of 4-nitro-N-propylbenzenesulfonamide (2.20 g, 9.00 mmol, 1.00 equiv) in EtOH (30.00 mL) and H₂O (10.00 mL) were added Fe (2.52 g, 45.00 mmol, 5.00 equiv) and NH₄Cl (4.78 g, 90.00 mmol, 10.00 equiv) at room temperature. The resulting mixture was stirred at 80° C. for 1 h. The mixture was cooled to room temperature, filtered, the solvent was removed in vacuo, then extracted with EA, dried over Na₂SO₄, filtered and dried to afford 4-amino-N-propylbenzenesulfonamide (1.70 g, 89.5%) as a white solid, which was used directly in the next step without further purification. [M+H]⁺=215.

### Step C. Synthesis of 2-(4-(7H-pyrrolo[2,3-d]pyrimidin-4-yl)piperazin-1-yl)-N-(4-(N-propylsulfamoyl)phenyl)acetamide (Compound 12)

To a stirred solution of 2-(4-(7H-pyrrolo[2,3-d]pyrimidin-4-yl)piperazin-1-yl)acetic acid (154 mg, 0.59 mmol, 1.00 equiv) and HATU (672.64 mg, 1.77 mmol, 3.00 equiv) in DMF (15 mL) were added pyridine (186.45 mg, 2.36 mmol, 4.00 equiv) and 4-amino-N-propylbenzenesulfonamide (126.26 mg, 0.59 mmol, 1.00 equiv) at room temperature. The resulting mixture was stirred at 40° C. for 18 h. The DMF was removed, extracted with EA, dried over Na₂SO₄, filtered. And the resulted solution was purified using Prep-HPLC with the following conditions: Column: RP-PREP-1 XBridge C18 Column, 19*150 mm, 5 μm; Mobile Phase A: Water (10 mmol/L NH₄HCO₃), Mobile Phase B: ACN; Flow rate: 15 mL/min; Gradient: 25% B to 45% B in 11 min; Wavelength: 214 nm. The title compound (38.00 mg, 14.1%) was obtained as a white solid. [M+H]⁺=458, H NMR (400 MHz, DMSO-d₆): δ 0.79 (3H, t, J=7.6 Hz), 1.33-1.39 (2H, m), 2.65-2.69 (6H, m), 3.25 (2H, s), 3.93-3.95 (4H, m), 6.62 (1H, t, J=1.6 Hz), 7.18 (1H, t, J=2.4 Hz), 7.44 (1H, t, J=6.0 Hz), 7.73 (2H, d, J=8.8 Hz), 7.85 (2H, d, J=8.8 Hz), 8.15 (1H, s), 10.16 (1H, s), 11.69 (1H, s).

### Example 13. Synthesis of 2-(4-(7H-pyrrolo[2,3-d]pyrimidin-4-yl)piperazin-1-yl)-N-(4-(N-ethylsulfamoyl)phenyl)acetamide (Compound 13)

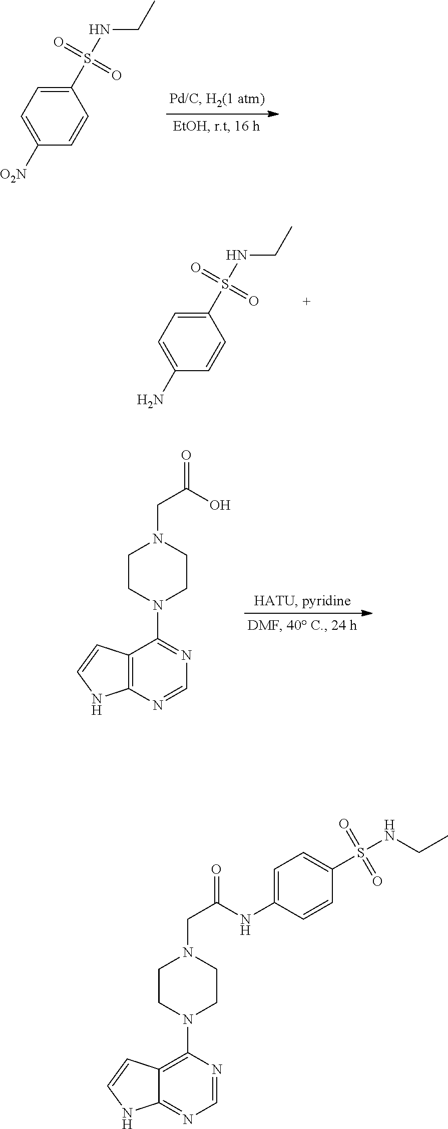 [SMILES: CCNS(=O)(=O)c1ccc([N+](=O)[O-])cc1 ; CCNS(=O)(=O)c1ccc(N)cc1 ; O=C(O)CN1CCN(c2ncnc3[nH]ccc23)CC1 ; CCNS(=O)(=O)c1ccc(NC(=O)CN2CCN(c3ncnc4[nH]ccc34)CC2)cc1]

### Step A. Synthesis of 4-amino-N-ethylbenzenesulfonamide

To a solution of N-ethyl-4-nitrobenzenesulfonamide (200 mg, 0.87 mmol, 1.00 equiv) in EtOH (10 mL) was added Pd/C (50 mg) at room temperature. The resulting mixture was stirred at room temperature for 16 h under H₂ (1 atm). The mixture was filtered and dried to afford 4-amino-N-ethylbenzenesulfonamide (170 mg, 97.7%) as a yellow solid, which was used directly in the next step without further purification. [M+H]⁺=201.

### Step B. Synthesis of 2-(4-(7H-pyrrolo[2,3-d]pyrimidin-4-yl)piperazin-1-yl)-N-(4-(N-ethylsulfamoyl)phenyl)acetamide (Compound 13)

To a stirred solution of 2-(4-(7H-pyrrolo[2,3-d]pyrimidin-4-yl)piperazin-1-yl)acetic acid (102 mg, 0.39 mmol, 1.00 equiv) and HATU (442 mg, 1.16 mmol, 3.00 equiv) in DMF (6 mL) were added pyridine (120.10 mg, 1.52 mmol, 4.00 equiv) and 4-amino-N-ethylbenzenesulfonamide (92.72 mg, 0.38 mmol, 1.00 equiv) at room temperature. The resulting mixture was stirred at 40° C. for 24 h. The DMF was removed, extracted with EA, dried over Na₂SO₄, filtered, and concentrated. The resulting product was purified using Prep-HPLC with the following conditions: RP-PREP-1 XBridge C18 Column, 19*150 mm, 5 μm; Mobile Phase A: Water (10 mmol/L NH₄HCO₃), Mobile Phase B: ACN; Flow rate: 15 mL/min; Gradient: 20% B to 40% B in 10 min; Wavelength: 214 nm. The title compound (31 mg, 18.0%) was obtained as a white solid. [M+H]=444, ¹H NMR (400 MHz, DMSO-d₆): δ 0.96 (3H, t, J=7.6 Hz), 2.65-2.67 (4H, m), 2.72-2.79 (2H, m), 3.25 (2H, s), 3.93-3.95 (4H, m), 6.62 (1H, t, J=1.6 Hz), 7.18 (1H, t, J=2.4 Hz), 7.42 (1H, t, J=5.6 Hz), 7.73 (2H, d, J=8.8 Hz), 7.86 (2H, d, J=8.8 Hz), 8.15 (1H, s), 10.17 (1H, s), 11.70 (1H, s).

### Example 14. Synthesis of 2-(4-(7H-pyrrolo[2,3-d]pyrimidin-4-yl)piperazin-1-yl)-N-methyl-N-(4-(N-methylsulfamoyl)phenyl)acetamide (Compound 14)

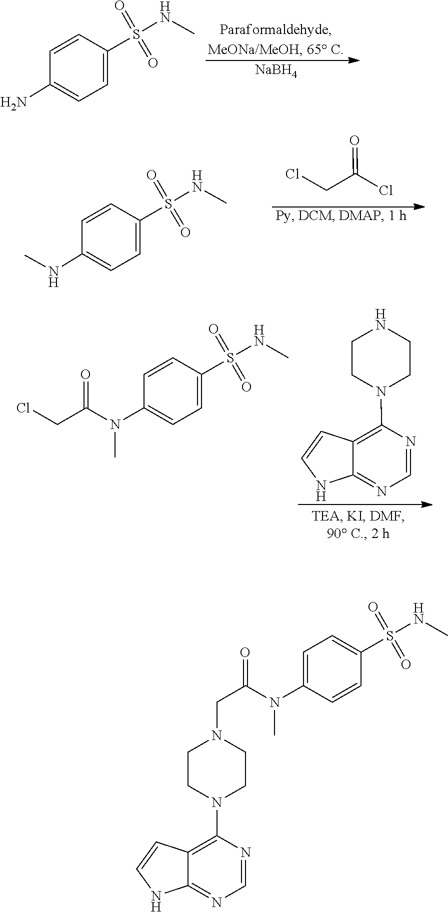

### Step A. Synthesis of N-methyl-4-(methylamino)benzenesulfonamide

To a solution of 4-amino-N-methylbenzenesulfonamide (0.20 g, 1.08 mmol, 1.00 equiv) and paraformaldehyde (0.29 g, 3.23 mmol, 3.00 equiv) in MeOH (10 mL) was added 30% MeONa/MeOH (0.58 g, 3.23 mmol, 3.00 equiv). The mixture was stirred at 65° C. for 16 h. Then the mixture was added NaBH₄ (0.12 g, 3.23 mmol, 3.00 equiv) slowly at R.T and then warmed to 65° C. for 2 h. LCMS showed the desired product was found. The mixture was concentrated in vacuo and the residue was added water (15 mL), extracted with EtOAc (3×10 mL). The combined organic layers were dried over Na₂SO₄, filtered and concentrated in vacuo to afford N-methyl-4-(methylamino)benzenesulfonamide (208 mg, 96%) as a yellow oil. [M+H]⁺=201.

### Step B. Synthesis of 2-chloro-N-methyl-N-(4-(N-methylsulfamoyl)phenyl)acetamide

To a stirred solution of N-methyl-4-(methylamino)benzenesulfonamide (150.00 mg, 0.75 mmol, 1.00 equiv) in DCM (10 mL) was added pyridine (119.00 mg, 1.50 mmol, 2.00 equiv), DMAP (10.00 mg, 0.08 mmol, 0.10 equiv) and then added dropwise a solution of 2-chloroacetyl chloride (85.00 mg, 0.75 mmol, 1.00 equiv) in DCM (1 mL) at room temperature. The resulting mixture was stirred at room temperature for 1 h. LCMS showed the reaction was finished. The mixture was diluted with H₂O and extracted with DCM. The combined organic layers were dried over Na₂SO₄, filtered and concentrated to afford crude 2-chloro-N-methyl-N-(4-(N-methylsulfamoyl)phenyl)acetamide (210 mg, 100%) as a yellow solid. [M+H]⁺=277.

### Step C. Synthesis of 2-(4-(7H-pyrrolo[2,3-d]pyrimidin-4-yl)piperazin-1-yl)-N-methyl-N-(4-(N-methylsulfamoyl)phenyl)acetamide (Compound 14)

To a solution of 2-chloro-N-methyl-N-(4-(N-methylsulfamoyl)phenyl)acetamide (100.00 mg, 0.36 mmol, 1.00 equiv) in EtOH/DMF (8 mL/5 mL) was added 4-(piperazin-1-yl)-7H-pyrrolo[2,3-d]pyrimidine (73.00 mg, 0.36 mmol, 1.00 equiv), TEA (182.00 mg, 1.80 mmol, 5.00 equiv) and KI (30.00 mg, 0.18 mmol, 0.50 equiv) at room temperature. The resulting mixture was stirred at 80° C. for 2 h. LCMS showed the desired product was found. The mixture was concentrated in vacuo. The residue was purified using Prep-HPLC with the following conditions: Column: RP-PREP-5 Xbridge C18 Column 5 m 19*150 mm; Mobile Phase A: Water (0.1% NH₄HCO₃), Mobile Phase B: ACN; Flow rate: 15 mL/min; Gradient: 10% B to 40% B in 11 min; Wavelength: 214 nm. The title compound (55 mg, 34%) was obtained as a white solid. [M+H]⁺=444. ¹H NMR (400 MHz, DMSO-d₆): δ 2.42-2.45 (7H, m), 3.15 (2H, s), 3.27 (3H, s), 3.71-3.73 (4H, m), 6.56 (1H, d, J=1.6 Hz), 7.16 (1H, t, J=2.8 Hz), 7.49 (1H, s), 7.60 (2H, d, J=8.4 Hz), 7.80 (2H, d, J=8.4 Hz), 8.11 (1H, s), 11.67 (1H, s).

### Example 17. Synthesis of 2-(4-(7H-pyrrolo[2,3-d]pyrimidin-4-yl)piperazin-1-yl)-N-(4-(N,N-diethylsulfamoyl)phenyl)acetamide (Compound 15

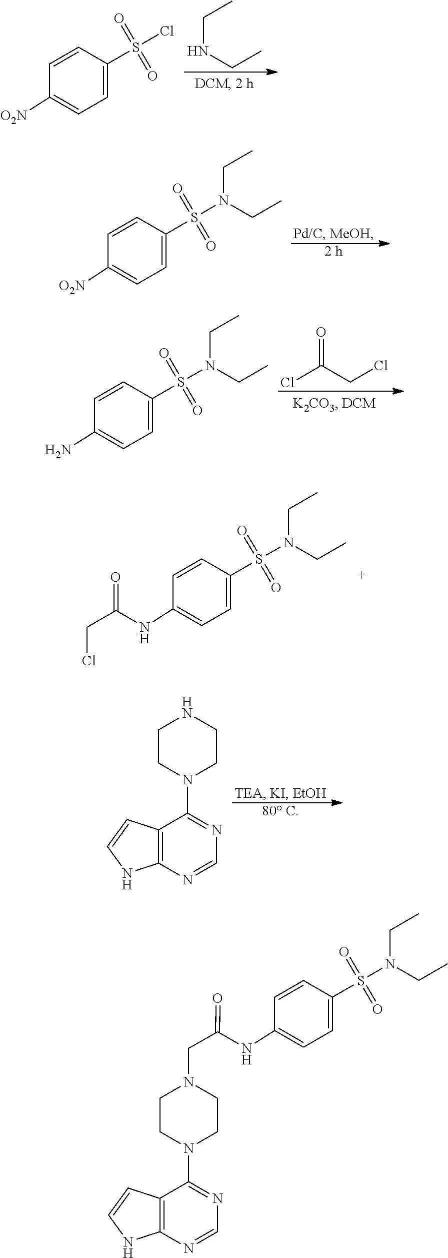

### Step A. Synthesis of N,N-diethyl-4-nitrobenzenesulfonamide

To a solution of dimethylamine in THF (15 mL, 2M) was added 4-nitrobenzenesulfonyl chloride (400 mg, 1.81 mmol, 1.00 equiv) at room temperature. The resulting mixture was stirred at room temperature for 2 h. The mixture was concentrated, diluted with NaHCO₃ solution, and extracted with EtOAc. The combined organic layers were dried over Na₂SO₄, filtered and concentrated to afford N,N-diethyl-4-nitrobenzenesulfonamide (450 mg, 97%) as a yellow solid. [M+H]⁺=259.

### Step B. Synthesis of 4-amino-N,N-diethylbenzenesulfonamide

To a stirred solution of N,N-diethyl-4-nitrobenzenesulfonamide (450 mg, 1.74 mmol, 1.00 equiv) in MeOH (15 mL) was added 10% Pd/C (100 mg) at room temperature under H₂ (1 atm). The resulting mixture was stirred at rt for 2 h. The mixture was filtered and concentrated to afford 4-amino-N,N-diethylbenzenesulfonamide (352 mg, 88%) a sa yellow solid. [M+H]⁺=229.

### Step C. Synthesis of 2-chloro-N-(4-(N,N-diethylsulfamoyl)phenyl)acetamide

To a stirring solution of 4-amino-N,N-diethylbenzenesulfonamide (350 mg, 1.54 mmol, 1.00 equiv) in DCM (10 mL) was added K₂CO₃ (425 mg, 3.08 mmol, 2.00 equiv), followed by 2-chloroacetyl chloride (173 mg, 1.54 mmol, 1.00 equiv) at room temperature. The resulting mixture was stirred at room temperature for 2 h. The mixture was diluted with H₂O and extracted with DCM. The combined organic layers were dried over Na₂SO₄, filtered and concentrated to afford 2-chloro-N-(4-(N,N-diethylsulfamoyl)phenyl)acetamide (325 mg, 70%) as a yellow solid. [M+H]⁺=305.

### Step D. Synthesis of 2-(4-(7H-pyrrolo[2,3-d]pyrimidin-4-yl)piperazin-1-yl)-N-(4-(N,N-diethylsulfamoyl)phenyl)acetamide (Compound 15)

To a solution of 2-chloro-N-(4-(N,N-diethylsulfamoyl)phenyl)acetamide (325 mg, 1.07 mmol, 1.00 equiv) in EtOH (10 mL) was added 4-(piperazin-1-yl)-7H-pyrrolo[2,3-d]pyrimidine (217 mg, 1.07 mmol, 1.00 equiv), TEA (216 mg, 2.14 mmol, 2.00 equiv) and KI (189 mg, 1.07 mmol, 1.0 equiv) at room temperature. The resulting mixture was stirred at 80° C. for 4 h. The resulted solution was evaporated and the residue purified by Prep-HPLC with the following conditions: Column: RP-PREP-5 Xbridge C18 Column, 19*150 mm, 5 μm; Mobile Phase A: Water (0.1% NH₄HCO₃), Mobile Phase B: ACN; Flow rate: 15 mL/min; Gradient: 35% B to 65% B in 10 min; Wavelength: 214 nm. The title compound (200 mg, 40%) was obtained as a white solid. [M+H]⁺=472. ¹H NMR (400 MHz, CDCl₃): δ 1.03 (t, J=6.8 Hz, 6H), 2.64-2.66 (m, 4H), 3.13 (q, J=7.2 Hz, 4H), 3.25 (2H, s), 3.92-3.95 (m, 4H), 6.62 (q, J=2.0 Hz, 1H), 7.18 (t, J=2.8 Hz, 1H), 7.74 (d, J=8.8 Hz, 2H), 7.87 (d, J=8.8 Hz, 2H), 8.14 (s, 1H), 10.19 (s, 1H), 11.69 (s, 1H).

### Example 16. Synthesis of 1-(2-(4-(7H-pyrrolo[2,3-d]pyrimidin-4-yl)piperazin-1-yl)acetyl)-N-phenylpiperidine-4-carboxamide (Compound 16)

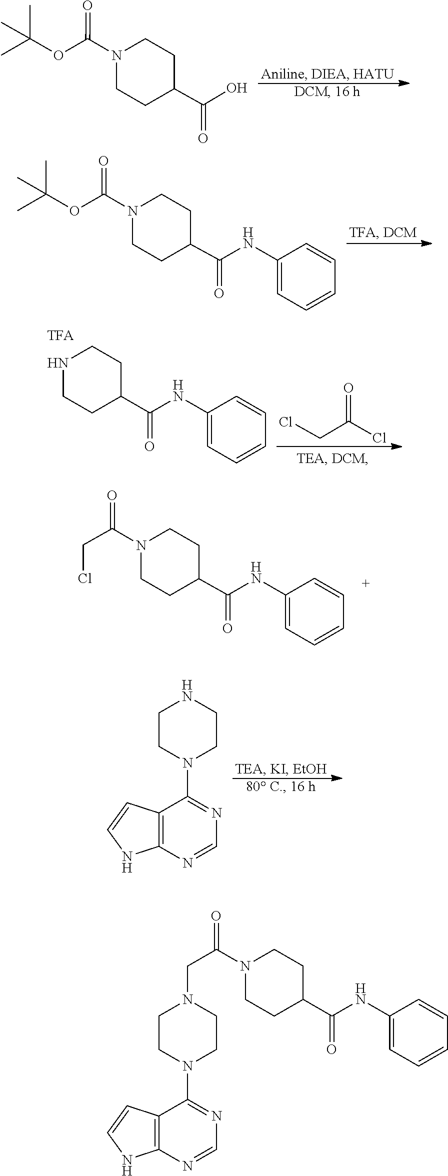

### Step A. Synthesis of tert-butyl 4-(phenylcarbamoyl)piperidine-1-carboxylate

To a stirred mixture of 1-(tert-butoxycarbonyl)piperidine-4-carboxylic acid (2.00 g, 8.73 mmol, 1.00 equiv) and HATU (3.98 g, 10.47 mmol, 1.20 equiv) in DCM (30 mL) was added DIEA (2.25 g, 17.46 mmol, 2.00 equiv) and aniline (812 mg, 8.73 mmol, 1.00 equiv). The resulting mixture was stirred at room temperature for 16 h. After concentrating in vacuo, the residue was purified by flash chromatography (EA/PE=25%) to afford tert-butyl 4-(phenylcarbamoyl)piperidine-1-carboxylate (1.20 g, 50%) as a white solid. [M+H]⁺=305.

### Step B. Synthesis of N-phenylpiperidine-4-carboxamide (TFA salt

To a solution of tert-butyl 4-(phenylcarbamoyl)piperidine-1-carboxylate (1.20 g, 3.90 mmol, 1.00 equiv) in DCM (12 mL) was added TFA (3 mL). The mixture was stirred at room temperature for 2 h. The mixture was concentrated in vacuo to afford N-phenylpiperidine-4-carboxamide (TFA salt; 1.00 g, 85%) as a yellow oil. [M+H]⁺=205.

### Step C. Synthesis of 1-(2-chloroacetyl)-N-phenylpiperidine-4-carboxamide

To a solution of N-phenylpiperidine-4-carboxamide (TFA salt; 1.00 g, 3.90 mmol, 1.00 equiv) in DCM (20 mL) was added TEA (799 mg, 7.89 mmol, 2.00 equiv) and 2-chloroacetyl chloride (529 mg, 4.68 mmol, 1.20 equiv). The mixture was stirred at room temperature for 2 h. After concentrated in vacuo, the residue was purified flash chromatography (EA/PE=30%) to afford 1-(2-chloroacetyl)-N-phenylpiperidine-4-carboxamide (600 mg, 55%) as a white solid. [M+H]⁺=281.

### Step D. Synthesis of 1-(2-(4-(7H-pyrrolo[2,3-d]pyrimidin-4-yl)piperazin-1-yl)acetyl)-N-phenylpiperidine-4-carboxamide (Compound 16)

To a solution of 1-(2-chloroacetyl)-N-phenylpiperidine-4-carboxamide (200 mg, 0.71 mmol, 1.00 equiv) in EtOH (10 mL) was added 4-(piperazin-1-yl)-7H-pyrrolo[2,3-d]pyrimidine (218 mg, 0.78 mmol, 1.10 equiv), TEA (287 mg, 2.84 mmol, 4.00 equiv) and KI (5 mg). The mixture was stirred at 80° C. for 16 h. After concentrated in vacuo, the crude was purified by Prep-HPLC with the following conditions (Column: XBridge C18 SN. Column, 19*150 mm, 5 μm; Mobile Phase A: Water (0.1% NH4HCO3), Mobile Phase B: ACN; Flow rate: 15 mL/min; Gradient: 30% B to 55% B in 8 min; Wavelength: 214 nm) to afford the title compound (100 mg, 32%) as a white solid. [M+H]⁺=448. ¹HNMR (400 MHz, DMSO-d₆): δ 1.44-1.45 (m, 1H), 1.47-1.48 (m, 1H), 1.66-1.68 (m, 2H), 2.54-2.67 (m, 6H), 3.04-3.16 (m, 2H), 3.44-3.48 (m, 1H), 3.86-3.87 (m, 4H), 4.12-4.17 (m, 1H), 4.39-4.42 (m, 1H), 6.61-6.62 (m, 1H), 7.02 (t, J=3.2 Hz, 1H), 7.18 (t, J=2.8 Hz, 1H), 7.26-7.29 (m, 2H), 7.59 (d, J=8.0 Hz, 2H), 8.14 (s, 1H), 9.90 (s, 1H), 11.68 (s, 1H).

### Example 17. Synthesis of 2-(4-(7H-pyrrolo[2,3-d]pyrimidin-4-yl)piperazin-1-yl)-N-(4-(N-isopropylsulfamoyl)phenyl)propenamide (Compound 17)

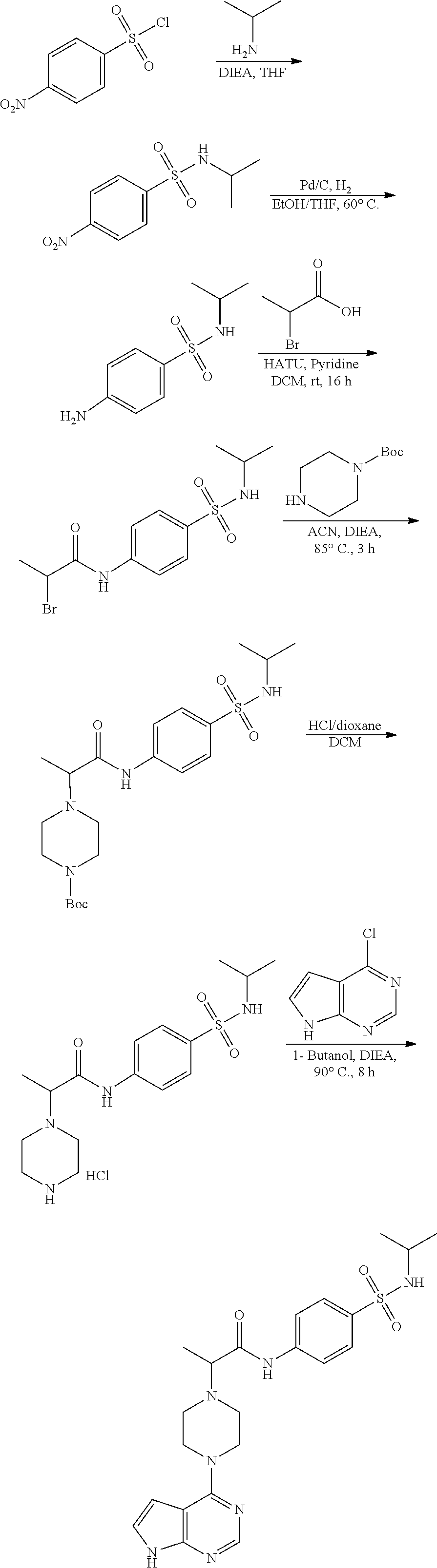

### Step A. Synthesis of N-isopropyl-4-nitrobenzenesulfonamide

To a stirred solution of 4-nitrobenzenesulfonyl chloride (3.00 g, 14.2 mmol, 1.00 equiv) in THF (30 mL) was slowly added propan-2-amine (1.26 g, 21.3 mmol, 1.50 equiv) and DIEA (2.33 g, 28.4 mmol, 2.00 equiv) at 0° C. The resulting mixture was stirred at room temperature for 16 h. The mixture was concentrated, diluted with water and extracted with EtOAc. The organic layer was dried over Na₂SO₄ and concentrated under reduced pressure to give N-isopropyl-4-nitrobenzenesulfonamide (1.70 g, 49%) as off-white solid. [M+H]⁺=245.1.

### Step B. Synthesis of 4-amino-N-isopropylbenzenesulfonamide

To a stirred solution of N-isopropyl-4-nitrobenzenesulfonamide (1.70 g, 6.96 mmol, 1.00 equiv) in EtOH/THF (25 mL/5 mL) was added 10% Pd/C (190 mg) at room temperature with H₂ (1 atm). The resulting mixture was stirred at 60° C. for 3 h. The mixture was filtered to afford 4-amino-N-isopropylbnzezenesulfonamide (1.30 g, 87.2%) as off-white solid. [M+H]⁺=215.1.

### Step C. Synthesis of 2-bromo-N-(4-(N-isopropylsulfamoyl)phenyl)propanamide

To a solution of 4-amino-N-isopropylbenzenesulfonamide (280 mg, 1.31 mmol, 1.00 equiv) and 2-bromopropanoic acid (298 mg, 1.96 mmol, 1.50 equiv) in DMF (10 mL) was added HATU (597 mg, 1.57 mmol, 1.20 equiv) and pyridine (310 mg, 3.93 mmol, 3.00 equiv). The resulting mixture was stirred at room temperature for 16 h. The mixture was extracted with EtOAc and washed with water. The organic layer was dried over Na₂SO₄ and concentrated. The mixture was purified by silica gel chromatography to afford 2-bromo-N-(4-(N-isopropylsulfamoyl)phenyl)propanamide (424 mg, 92%) as white solid. [M+H]⁺=349.0.

### Step D. Synthesis of tert-butyl 4-(1-((4-(N-isopropylsulfamoyl)phenyl)amino)-1-oxopropan-2-yl)piperazine-1-carboxylate

To a solution of 2-bromo-N-(4-(N-isopropylsulfamoyl)phenyl)propanamide (424 mg, 1.21 mmol, 1.00 equiv) and tert-butyl piperazine-1-carboxylate (270 mg, 1.45 mmol, 1.20 equiv) in ACN (15 mL) was added DIEA (315 mg, 2.42 mmol, 2.00 equiv). The mixture was stirred at 85° C. for 2 h. The mixture was concentrated. The residue was purified by silica gel chromatography to afford tert-butyl 4-(1-oxo-1-((4-(piperidin-1-ylsulfonyl)phenyl)amino)propan-2-yl) piperazine-1-carboxylate (498 mg, 90%) as an off-white solid. [M+H]+=455.2.

### Step E. Synthesis of N-(4-(N-isopropylsulfamoyl)phenyl)-2-(piperazin-1-yl)propanamide (HCl salt)

To a solution of tert-butyl 4-(1-oxo-1-((4-(piperidin-1-ylsulfonyl)phenyl)amino)propan-2-yl)piperazine-1-carboxylate (120 mg, 0.26 mmol, 1.00 equiv) in 2 mL DCM was added 1.5 mL HCl (4M in dioxane). The mixture was stirred at room temperature for 2 h. The mixture was concentrated to afford N-(4-(N-isopropylsulfamoyl)phenyl)-2-(piperazin-1-yl)propanamide (HCl salt; 110 mg, >100%) as an off-white solid. [M+H]⁺=355.2.

### Step F. Synthesis of 2-(4-(7H-pyrrolo[2,3-d]pyrimidin-4-yl)piperazin-1-yl)-N-(4-(N-isopropylsulfamoyl)phenyl)propenamide (Compound 17)

To a solution of N-(4-(N-isopropylsulfamoyl)phenyl)-2-(piperazin-1-yl)propanamide (HCl salt) (80 mg, 0.19 mmol, 1.00 equiv) and 4-chloro-7H-pyrrolo[2,3-d]pyrimidine (121 mg, 0.21 mmol, 1.10 equiv) in 1-butanol (10 mL) was added DIEA (74 mg, 0.57 mmol, 3.00 equiv). The mixture was stirred at 90° C. for 8 h. The mixture was concentrated. The residue was purified by using Prep-HPLC with the following conditions: Column: XBridge Prep C18 Column, 19*150 mm, 5 μm; Mobile Phase A: Water (0.10% NH₄HCO₃), Mobile Phase B: ACN; Flow rate: 15 mL/min; Gradient: 15% B to 70% B in 10 min, Wavelength: 214 nm. The title compound (15.00 mg, 11%) was obtained as a white solid.

### Example 18. Synthesis of (R)-2-(4-(7H-pyrrolo[2,3-d]pyrimidin-4-yl)piperazin-1-yl)-N-(4-(N-isopropylsulfamoyl)phenyl)propanamide (Compound 18)

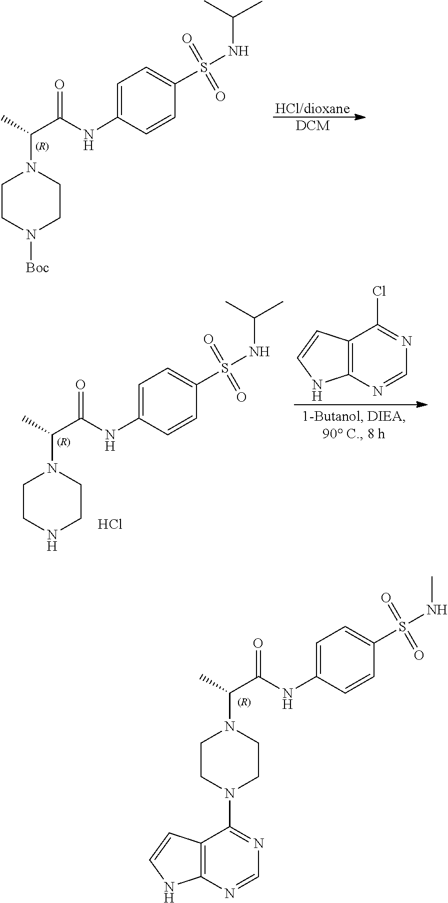

### Step A. Isolation of tert-butyl (R)-4-(1-((4-(N-isopropylsulfamoyl)phenyl)amino)-1-oxopropan-2-yl)piperazine-1-carboxylate by chiral separation

Tert-butyl 4-(1-((4-(N-isopropylsulfamoyl)phenyl)amino)-1-oxopropan-2-yl)piperazine-1-carboxylate (480 mg) was separated by chiral HPLC to afford two enantiomers. The R-enantiomer was obtained in a 200 mg quantity.

### Step B. Synthesis of (R)—N-(4-(N-isopropylsulfamoyl)phenyl)-2-(piperazin-1-yl)propanamide (HCl salt)

To a solution of tert-butyl (R)-4-(1-((4-(N-isopropylsulfamoyl)phenyl)amino)-1-oxopropan-2-yl)piperazine-1-carboxylate (Step A; 120 mg, 0.26 mmol, 1.00 equiv) in 2 mL DCM was added 1.5 mL HCl. The mixture was stirred at room temperature for 2 h. The mixture was concentrated to afford (R)—N-(4-(N-isopropylsulfamoyl)phenyl)-2-(piperazin-1-yl)propenamide (HCl salt; 110 mg, >100%) as an off-white solid. [M+H]⁺=355.2.

### Step C. Synthesis of (R)-2-(4-(7H-pyrrolo[2,3-d]pyrimidin-4-yl)piperazin-1-yl)-N-(4-(N-isopropylsulfamoyl)phenyl)propanamide (Compound 18)

To a solution of (R)-2-(piperazin-1-yl)-N-(4-(piperidin-1-ylsulfonyl)phenyl)propanamide (HCl salt) (80 mg, 0.19 mmol, 1.00 equiv) and 4-chloro-7H-pyrrolo[2,3-d]pyrimidine (121 mg, 0.21 mmol, 1.10 equiv) in 1-butanol (10 mL) was added DIEA (74 mg, 0.57 mmol, 3.00 equiv). The mixture was stirred at 90° C. for 8 h. The mixture was concentrated. The residue was purified by Prep-HPLC with the following conditions: Column: XBridge Prep C18 Column, 19*150 mm, 5 μm; Mobile Phase A: Water (0.1% NH₄ HCO₃), Mobile Phase B: ACN; Flow rate: 15 mL/min; Gradient: 15% B to 70% B in 10 min, Wavelength: 214 nm. (R)-2-(4-(7H-pyrrolo[2,3-d]pyrimidin-4-yl)piperazin-1-yl)-N-(4-(N-isopropylsulfamoyl)phenyl)propanamide (15 mg, 11%) was obtained as a white solid. [M+H]=472.2. ¹H NMR (400 MHz, DMSO-d₆): δ 0.93 (d, J=6.8 Hz, 6H), 1.23 (d, J=7.2 Hz, 3H), 2.60-2.75 (m, 4H), 3.14-3.25 (m, 1H), 3.35-3.42 (m, 1H), 3.89-3.94 (m, 4H), 6.61 (s, 1H), 7.17 (s, 1H), 7.43 (d, J=8.0 Hz, 1H), 7.74 (d, J=8.8 Hz, 2H), 7.85 (d, J=8.4 Hz, 2H), 8.13 (s, 1H), 10.21 (s, 1H), 11.68 (s, 1H).

### Example 19. Synthesis of (S)-2-(4-(7H-pyrrolo[2,3-d]pyrimidin-4-yl)piperazin-1-yl)-N-(4-(N-isopropylsulfamoyl)phenyl)propenamide (Compound 19)

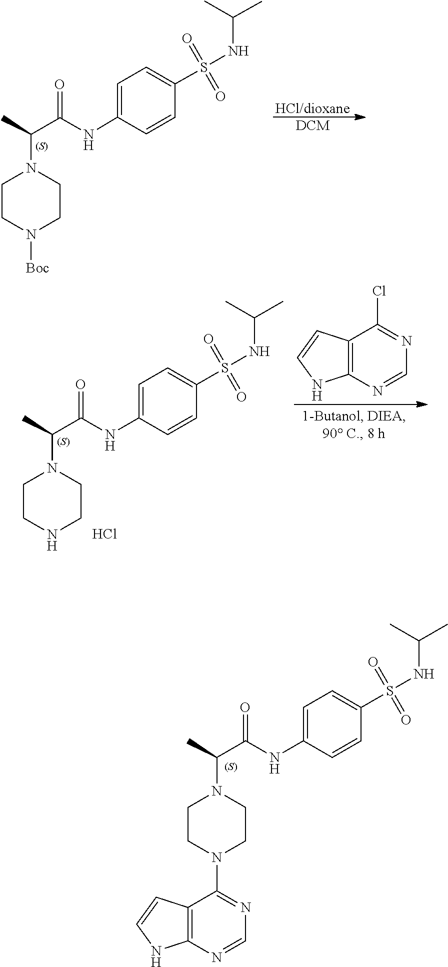

### Step A. Synthesis of (S)—N-(4-(N-isopropylsulfamoyl)phenyl)-2-(piperazin-1-yl)propanamide (HCl salt)

To a solution of tert-butyl-(S)-4-(1-((4-(N-isopropylsulfamoyl)phenyl)amino)-1-oxopropan-2-yl)piperazine-1-carboxylate (Example 13, Step A; 120 mg, 0.26 mmol, 1.00 equiv) in 2 mL DCM was added 1.5 mL HCl. The mixture was stirred at room temperature for 2 h. The mixture was concentrated to afford (S)—N-(4-(N-isopropylsulfamoyl)phenyl)-2-(piperazin-1-yl)propanamide (HCl salt; 70 mg, >100%) as a white solid. [M+H]+=355.2.

### Step B. Synthesis of (S)-2-(4-(7H-pyrrolo[2,3-d]pyrimidin-4-yl)piperazin-1-yl)-N-(4-(N-isopropylsulfamoyl)phenyl)propenamide (Compound 19)

To a solution of (S)—N-(4-(N-isopropylsulfamoyl)phenyl)-2-(piperazin-1-yl)propanamide (HCl salt) (70 mg, 0.20 mmol, 1.00 equiv) and 4-chloro-7H-pyrrolo[2,3-d]pyrimidine (31 mg, 0.20 mmol, 1.00 equiv) in 1-butanol (10 mL) was added DIEA (78 mg, 0.60 mmol, 3.00 equiv). The mixture was stirred at 90° C. for 8 h. The mixture was concentrated. The residue was purified by Prep-HPLC with the following conditions: Column: XBridge Prep C18 Column, 19*150 mm, 5 μm; Mobile Phase A: Water (0.1% NH₄ HCO₃), Mobile Phase B: ACN; Flow rate: 15 mL/min; Gradient: 15% B to 70% B in 10 min, Wavelength: 214 nm. The title compound (18 mg, 19%) was obtained as a white solid. [M+H]⁺=472.2. ¹H NMR (400 MHz, DMSO-d₆): δ 0.93 (d, J=6.4 Hz, 6H), 1.23 (d, J=6.8 Hz, 3H), 2.59-2.74 (m, 4H), 3.14-3.26 (m, 1H), 3.34-3.42 (m, 1H), 3.90-3.94 (m, 4H), 6.05 (s, 1H), 7.17 (s, 1H), 7.73 (d, J=8.8 Hz, 2H), 7.85 (d, J=8.8 Hz, 2H), 8.13 (s, 1H), 10.21 (s, 1H), 11.66 (s, 1H).

### Example 20. Synthesis of 2-(4-(7H-pyrrolo[2,3-d]pyrimidin-4-yl)piperazin-1-yl)-N-(4-(piperidin-1-ylsulfonyl)phenyl)propenamide (Compound 20)

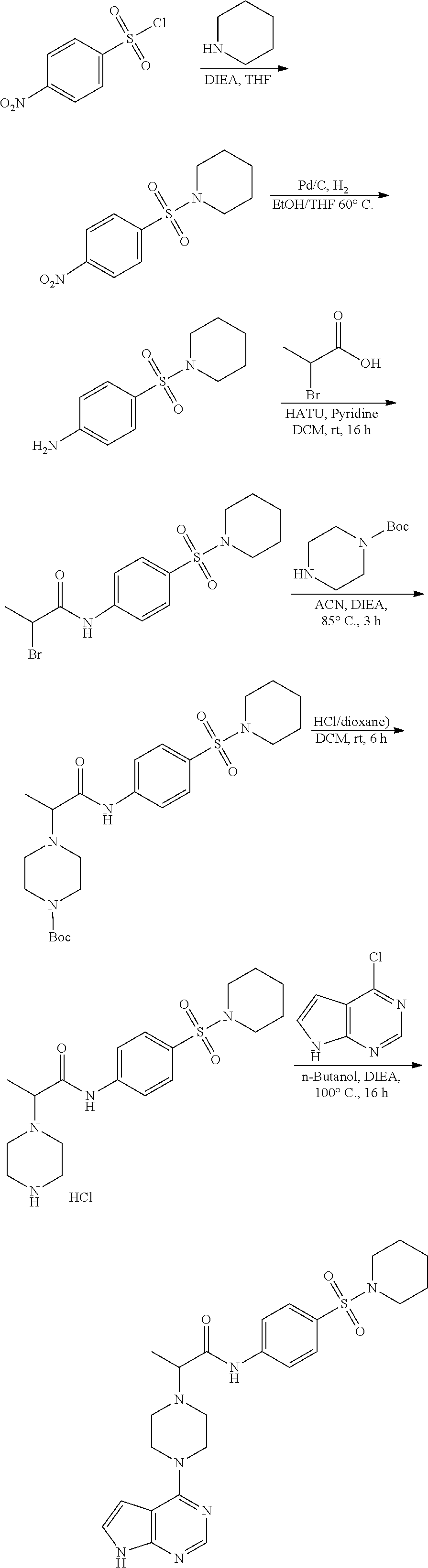

### Step A. Synthesis of 1-((4-nitrophenyl)sulfonyl)piperidine

To a stirred solution of 4-nitrobenzenesulfonyl chloride (2.00 g, 9.02 mmol, 1.00 equiv) in THF (30 mL) was slowly added piperidine (1.15 g, 13.54 mmol, 1.50 equiv) and DIEA (2.33 g, 18.04 mmol, 2.00 equiv) at 0° C. The resulting mixture was stirred at room temperature for 16 h. The mixture was concentrated, diluted with water and extracted with EtOAc. The organic layer was dried over Na₂SO₄ and concentrated under reduced pressure to give 1-((4-nitrophenyl)sulfonyl)piperidine (1.97 g, 80.9%) as an off-white solid. [M+H]⁺=271.1.

### Step B. Synthesis of 4-(piperidin-1-ylsulfonyl)aniline

To a stirred solution of 1-((4-nitrophenyl)sulfonyl)piperidine (1.97 g, 7.30 mmol, 1.00 equiv) in EtOH/THF (25 mL/5 mL) was added 10% Pd/C (197 mg) at room temperature under H₂ (1 atm). The resulting mixture was stirred at 60° C. for 3 h. The mixture was filtered and concentrated in vacuo to afford 4-(piperidin-1-ylsulfonyl)aniline (1.07 g, 61%) as an off-white solid. [M+H]⁺=241.1.

### Step C. Synthesis of 2-bromo-N-(4-(piperidin-1-ylsulfonyl)phenyl)propanamide

To a solution of 4-(piperidin-1-ylsulfonyl)aniline (400 mg, 1.67 mmol, 1.00 equiv) and 2-bromopropanoic acid (306 mg, 2.0 mmol, 1.20 equiv) in DMF (10 mL) was added HATU (760 mg, 2.00 mmol, 1.20 equiv) and pyridine (196 mg, 5.01 mmol, 3.00 equiv). The resulting mixture was stirred at room temperature for 16 h. The mixture was extracted with EtOAc and washed with water. The organic layer was dried over Na₂SO₄, filtered and concentrated in vacuo. The mixture was purified by silica gel chromatography to give 2-bromo-N-(4-(piperidin-1-ylsulfonyl)phenyl)propanamide (500 mg, 80%) as a white solid. [M+H]⁺=375.0.

### Step D. Synthesis of tert-butyl 4-(1-oxo-1-((4-(piperidin-1-ylsulfonyl)phenyl)amino) propan-2-yl)piperazine-1-carboxylate

To a solution of 2-bromo-N-(4-(piperidin-1-ylsulfonyl)phenyl)propanamide (460 mg, 1.23 mmol, 1.00 equiv) and tert-butyl piperazine-1-carboxylate (274 mg, 1.47 mmol, 1.20 equiv) in ACN(15 mL) was added DIEA (316 mg, 2.45 mmol, 2.00 equiv). The mixture was stirred at 85° C. for 2 h. The LCMS showed the reaction was finished. The mixture was concentrated and the residue was purified by silica gel chromatography to afford tert-butyl 4-(1-oxo-1-((4-(piperidin-1-ylsulfonyl)phenyl)amino)propan-2-yl)piperazine-1-carboxylate (533 mg, 90%) as an off-white solid. [M+H]⁺=481.2.

### Step E. Synthesis of 2-(piperazin-1-yl)-N-(4-(piperidin-1-ylsulfonyl)phenyl) propanamide

To a solution of tert-butyl 4-(1-oxo-1-((4-(piperidin-1-ylsulfonyl)phenyl)amino)propan-2-yl)piperazine-1-carboxylate (100 mg, 0.21 mmol, 1.00 equiv) in 4 mL DCM was added 1.5 mL HCl (4M in dioxane). The mixture was stirred at room temperature for 6 h. The mixture was concentrated to get 2-(piperazin-1-yl)-N-(4-(piperidin-1-ylsulfonyl)phenyl)propanamide (HCl salt; 80 mg, 91.6%) as a white solid. [M+H]⁺=381.1.

### Step F. Synthesis of 2-(4-(7H-pyrrolo[2,3-d]pyrimidin-4-yl)piperazin-1-yl)-N-(4-(piperidin-1-ylsulfonyl)phenyl)propenamide (Compound 20)

To a solution of 2-(piperazin-1-yl)-N-(4-(piperidin-1-ylsulfonyl)phenyl)propanamide (80 mg, 0.19 mmol, 1.00 equiv) and 4-chloro-7H-pyrrolo[2,3-d]pyrimidine (121 mg, 0.21 mmol, 1.10 equiv) in 1-butanol (10 mL) was added DIEA (74 mg, 0.57 mmol, 3.00 equiv). The mixture was stirred at 100° C. for 16 h. The mixture was concentrated. The residue was purified by Prep-HPLC with the following conditions: Column: XBridge Prep C18 Column, 19*150 mm, 5 μm; Mobile Phase A: Water (0.1% NH₄HCO₃), Mobile Phase B: ACN; Flow rate: 15 mL/min; Gradient: 20% B to 75% B in 10 min, Wavelength: 214 nm. The title compound (89 mg, 93%) was obtained as a white solid.

### Example 21. Synthesis of (R)-2-(4-(7H-pyrrolo[2,3-d]pyrimidin-4-yl)piperazin-1-yl)-N-(4-(piperidin-1-ylsulfonyl)phenyl)propenamide (Compound 21)

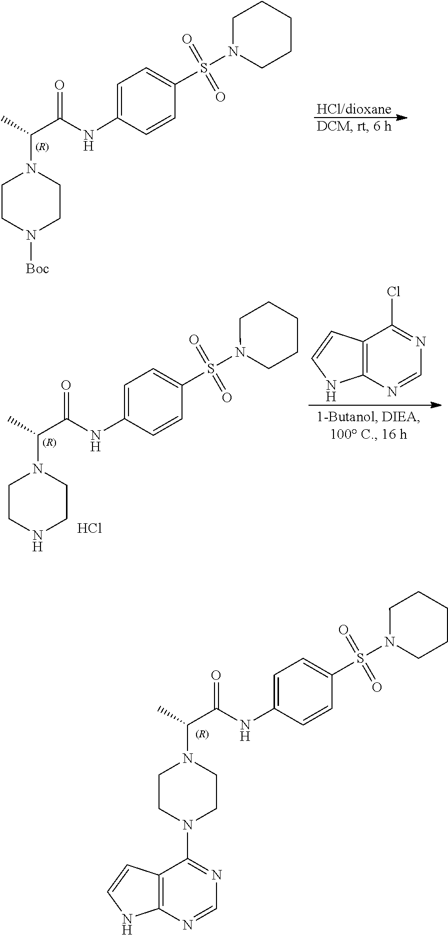

### Step A. Chiral separation of racemic tert-butyl 4-(1-oxo-1-((4-(piperidin-1-ylsulfonyl)phenyl)amino)propan-2-yl)piperazine-1-carboxylate

Racemic tert-butyl 4-(1-oxo-1-((4-(piperidin-1-ylsulfonyl)phenyl)amino)propan-2-yl)piperazine-1-carboxylate (530 mg) was separated by chiral HPLC to obtain two enantiomers: the (S)-enantiomer (234 mg) and the (R)-enantiomer (220 mg).

### Step B. Synthesis of (R)-2-(piperazin-1-yl)-N-(4-(piperidin-1-ylsulfonyl)phenyl) propanamide (HCl salt)

To a solution of tert-butyl (R)-4-(1-oxo-1-((4-(piperidin-1-ylsulfonyl)phenyl)amino)propan-2-yl)piperazine-1-carboxylate (100 mg, 0.21 mmol, 1.00 equiv) in 4 mL DCM was added 1.5 mL HCl (4M in dioxane). The mixture was stirred at room temperature for 6 h. The mixture was concentrated to afford (R)-2-(piperazin-1-yl)-N-(4-(piperidin-1-ylsulfonyl)phenyl)propanamide (HCl salt; 80 mg, 91.6%) as a white solid. [M+H]⁺=381.1.

### Step C. Synthesis of (R)-2-(4-(7H-pyrrolo[2,3-d]pyrimidin-4-yl)piperazin-1-yl)-N-(4-(piperidin-1-ylsulfonyl)phenyl)propenamide (Compound 21)

To a solution of (R)-2-(piperazin-1-yl)-N-(4-(piperidin-1-ylsulfonyl)phenyl)propanamide (HCl salt; 80 mg, 0.19 mmol, 1.00 equiv) and 4-chloro-7H-pyrrolo[2,3-d]pyrimidine (121 mg, 0.21 mmol, 1.10 equiv) in 1-butanol (10 mL) was added DIEA (74 mg, 0.57 mmol, 3.00 equiv). The mixture was stirred at 100° C. for 16 h. The mixture was concentrated. The residue was purified by Prep-HPLC with the following conditions: Column: XBridge Prep C18 Column, 19*150 mm, 5 μm; Mobile Phase A: Water (0.1% NH₄ HCO₃), Mobile Phase B: ACN; Flow rate: 15 mL/min; Gradient: 20% B to 75% B in 10 min, Wavelength: 214 nm. The title compound (89 mg, 93%) was obtained as a white solid. [M+H]⁺=498.2. ¹H NMR (400 MHz, DMSO-d₆): δ 1.24 (t, J=6.8 Hz, 3H), 1.34-1.35 (m, 2H), 1.52-1.53 (m, 4H), 2.60-2.73 (m, 4H), 2.85 (t, J=5.2 Hz, 4H), 3.37-3.42 (m, 1H), 3.92 (t, J=4.8 Hz, 4H), 6.61 (d, J=2.4 Hz, 1H), 7.18 (t, J=3.2 Hz, 1H), 7.67 (d, J=8.8 Hz, 2H), 7.92 (d, J=8.8 Hz, 2H), 8.14 (s, 1H), 10.28 (s, 1H), 11.68 (s, 1H).

### Example 22. Synthesis of (S)-2-(4-(7H-pyrrolo[2,3-d]pyrimidin-4-yl)piperazin-1-yl)-N-(4-(piperidin-1-ylsulfonyl)phenyl)propenamide (Compound 22)

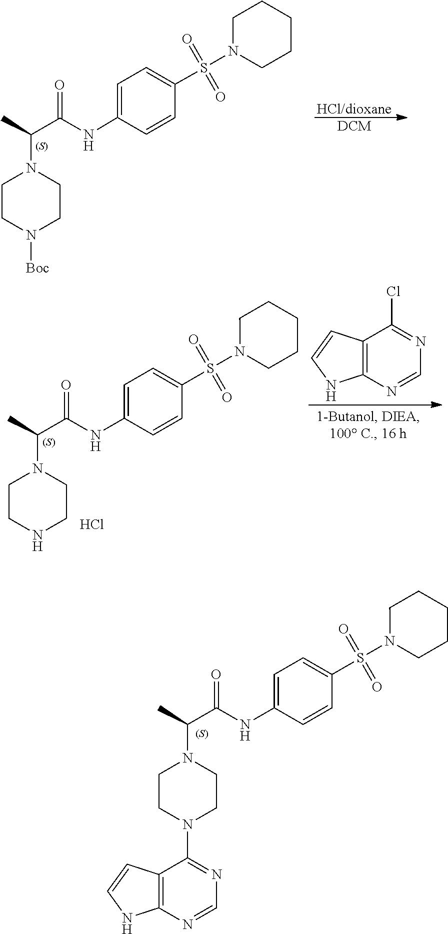

### Step A. Synthesis of (S)-2-(piperazin-1-yl)-N-(4-(piperidin-1-ylsulfonyl)phenyl)propanamide (HCl salt)

To a solution of tert-butyl (S)-4-(1-oxo-1-((4-(piperidin-1-ylsulfonyl)phenyl)amino)propan-2-yl)piperazine-1-carboxylate (Example 26, Step A; 100 mg, 0.21 mmol, 1.00 equiv) in 4 mL DCM was added 1.5 mL HCl (4M in dioxane). The mixture was stirred at room temperature for 6 h. The mixture was concentrated to afford (S)-2-(piperazin-1-yl)-N-(4-(piperidin-1-ylsulfonyl)phenyl)propanamide (HCl salt; 80 mg, 92%) as a white solid. [M+H]⁺=381.1.

### Step B. Synthesis of (S)-2-(4-(7H-pyrrolo[2,3-d]pyrimidin-4-yl)piperazin-1-yl)-N-(4-(piperidin-1-ylsulfonyl)phenyl)propenamide (Compound 22)

To a solution of (S)-2-(piperazin-1-yl)-N-(4-(piperidin-1-ylsulfonyl)phenyl)propanamide (HCl salt; 80 mg, 0.19 mmol, 1.00 equiv) and 4-chloro-7H-pyrrolo[2,3-d]pyrimidine (121 mg, 0.21 mmol, 1.10 equiv) in 1-butanol (10 mL) was added DIEA (74 mg, 0.57 mmol, 3.00 equiv). The mixture was stirred at 100° C. for 16 h. LCMS showed the reaction was complete. The mixture was concentrated and the residue purified by Prep-HPLC with the following conditions: Column XBridge Prep C18 Column, 19*150 mm, 5 μm; Mobile Phase A: Water (0.1% NH₄HCO₃), Mobile Phase B: ACN; Flow rate: 15 mL/min; Gradient: 20% B to 75% B in 10 min, Wavelength: 214 nm. The title compound (81 mg, 84%) was obtained as a white solid. [M+H]⁺=498.2. ¹H NMR (400 MHz, DMSO-d₆): δ 1.24 (t, J=6.8 Hz, 3H), 1.34-1.36 (m, 2H), 1.52-1.53 (m, 4H), 2.60-2.73 (m, 4H), 2.85 (t, J=5.2 Hz, 4H), 3.37-3.42 (m, 1H), 3.92 (t, J=4.4 Hz, 4H), 6.61 (d, J=2.0 Hz, 1H), 7.18 (t, J=3.2 Hz, 1H), 7.67 (d, J=8.8 Hz, 2H), 7.92 (d, J=8.4 Hz, 2H), 8.14 (s, 1H), 10.28 (s, 1H), 11.68 (s, 1H).

### Example 23. Synthesis of 1-[4-(3-methylpiperidine-1-carbonyl)piperidin-1-yl]-2-(4-[1H-pyrrolo[2,3-d]pyrimidin-4-yl]piperazin-1-yl)ethan-1-one (Compound 23)

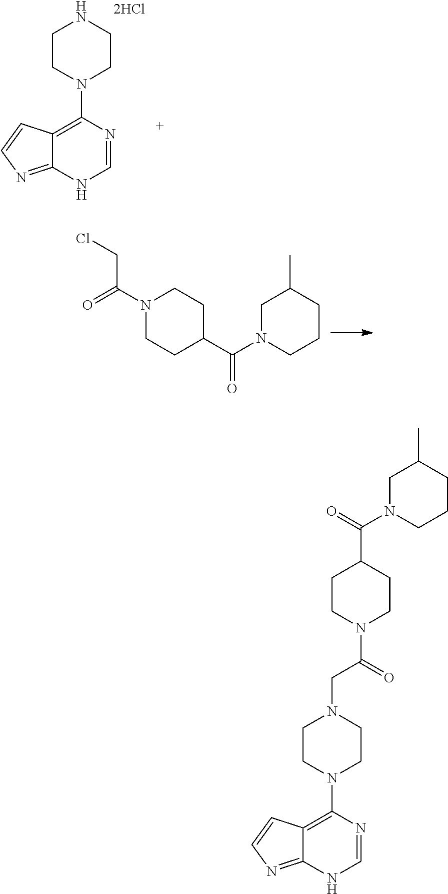

To 1-[1H-pyrrolo[2,3-d]pyrimidin-4-yl]piperazine dihydrochloride (67 mg, 0.244 mmol) in dry DMSO (approximately 0.5 ml) was added 2-chloro-1-[4-(3-methylpiperidine-1-carbonyl)piperidin-1-yl]ethan-1-one (71 mg, 0.248 mmol), diisopropylamine (122 mg, 1.206 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 30% to 80% of 0.1% NH3/methanol in 0.1% aqueous NH₃. The title product was obtained in 59% yield (65.2 mg).

### Example 24. Synthesis of N-methyl-N-(propan-2-yl)-1-[2-(4-{1H-pyrrolo[2,3-d]pyrimidin-4-yl}piperazin-1-yl)acetyl]piperidine-4-carboxamide (Compound 24)

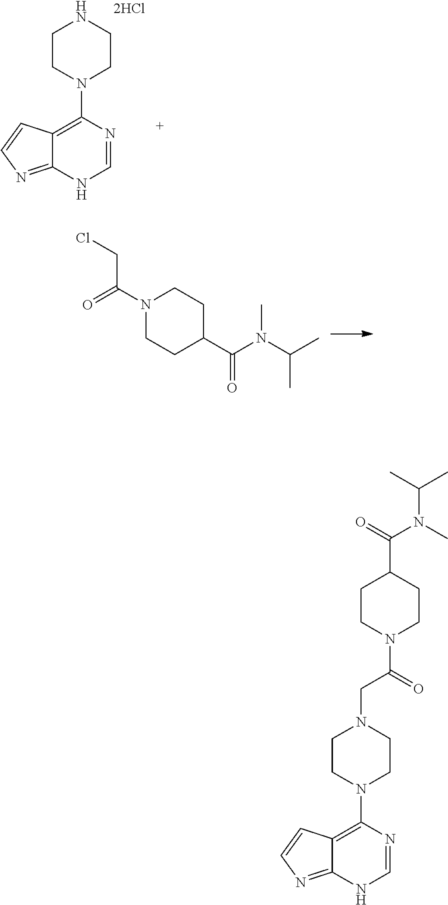

To 1-[1H-pyrrolo[2,3-d]pyrimidin-4-yl]piperazine dihydrochloride (70 mg, 0.254 mmol) in dry DMSO (approximately 0.5 ml) was added 1-(2-chloroacetyl)-N-methyl-N-(propan-2-yl)piperidine-4-carboxamide (74 mg, 0.284 mmol), diisopropylamine (136 mg, 1.345 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 20% to 70% of 0.1% NH₃/methanol in 0.1% aqueous NH₃. The title product was obtained in 42% yield (45.3 mg).

### Example 25. Synthesis of N-{4-[(pyrimidin-2-yl)sulfamoyl]phenyl}-2-(4-{1H-pyrrolo[2,3-d]pyrimidin-4-yl}piperazin-1-yl)acetamide (Compound 25)

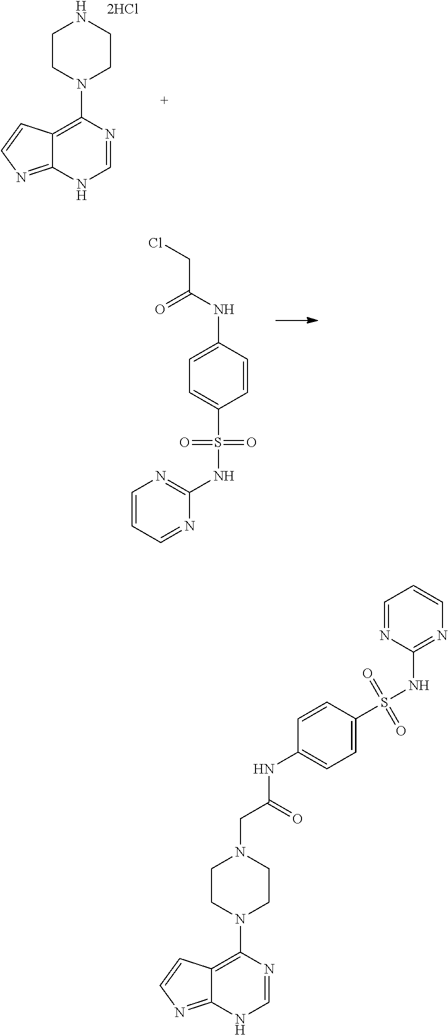 [SMILES: c1cc2c(N3CCNCC3)nc[nH]c-2n1 ; O=C(CCl)Nc1ccc(S(=O)(=O)Nc2ncccn2)cc1 ; O=C(CN1CCN(c2nc[nH]c3nccc2-3)CC1)Nc1ccc(S(=O)(=O)Nc2ncccn2)cc1]

To 1-[1H-pyrrolo[2,3-d]pyrimidin-4-yl]piperazine dihydrochloride (59 mg, 0.214 mmol) in dry DMSO (approximately 0.5 ml) was added 2-chloro-N-{4-[(pyrimidin-2-yl)sulfamoyl]phenyl}acetamide (70 mg, 0.215 mmol), diisopropylamine (106 mg, 1.048 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 30% to 80% of 0.1% NH3/methanol in 0.1% aqueous NH₃. The title product was obtained in 16% yield (16.8 mg).

### Example 26. Synthesis of 1-(4-benzylpiperidin-1-yl)-2-(4-{1H-pyrrolo[2,3-d]pyrimidin-4-yl}piperazin-1-yl)ethan-1-one (Compound 26)

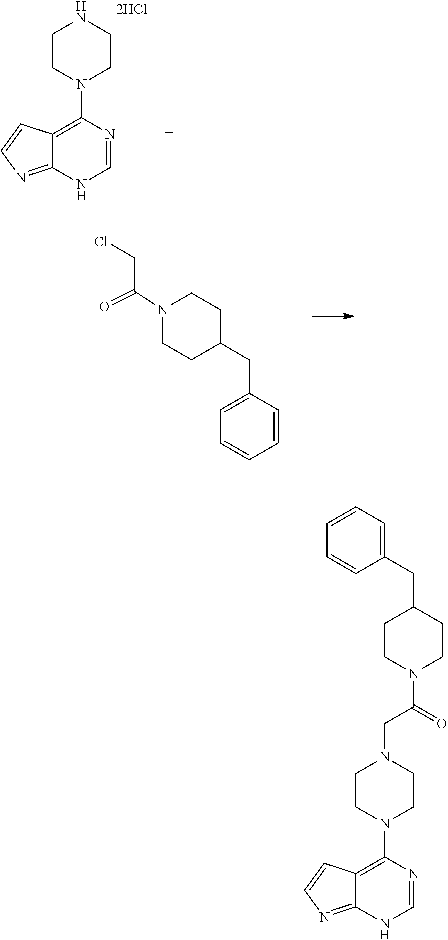 [SMILES: c1cc2c(N3CCNCC3)nc[nH]c-2n1 ; O=C(CCl)N1CCC(Cc2ccccc2)CC1 ; O=C(CN1CCN(c2nc[nH]c3nccc2-3)CC1)N1CCC(Cc2ccccc2)CC1]

To 1-[1H-pyrrolo[2,3-d]pyrimidin-4-yl]piperazine dihydrochloride (72 mg, 0.262 mmol) in dry DMSO (approximately 0.5 ml) was added 1-(4-benzylpiperidin-1-yl)-2-chloroethan-1-one (67 mg, 0.267 mmol), diisopropylamine (131 mg, 1.295 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 50% to 100% of 0.1% NH3/methanol in 0.10% aqueous NH₃. The title product was obtained in 49% yield (53.9 mg).

### Example 27. Synthesis of 2-(4-{1H-pyrrolo[2,3-d]pyrimidin-4-yl}piperazin-1-yl)-N-(3-sulfamoylphenyl)acetamide (Compound 27)

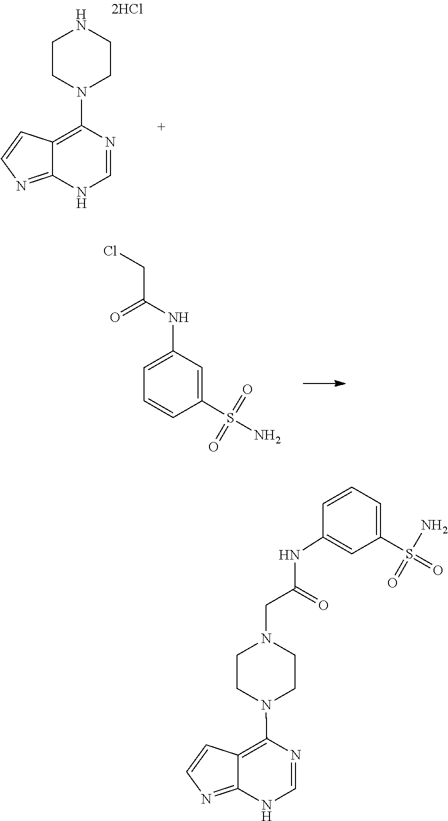 [SMILES: c1cc2c(N3CCNCC3)nc[nH]c-2n1 ; NS(=O)(=O)c1cccc(NC(=O)CCl)c1 ; NS(=O)(=O)c1cccc(NC(=O)CN2CCN(c3nc[nH]c4nccc3-4)CC2)c1]

To 1-[1H-pyrrolo[2,3-d]pyrimidin-4-yl]piperazine dihydrochloride (72 mg, 0.262 mmol) in dry DMSO (approximately 0.5 ml) was added 2-chloro-N-(3-sulfamoylphenyl)acetamide (69 mg, 0.278 mmol), diisopropylamine (138 mg, 1.365 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 20% to 70% of 0.1% NH₃/methanol in 0.1% aqueous NH₃. The title product was obtained in 33% yield (36 mg).

### Example 28. Synthesis of N-phenyl-4-[2-(4-{1H-pyrrolo[2,3-d]pyrimidin-4-yl}piperazin-1-yl)acetyl]piperazine-1-carboxamide (Compound 28)

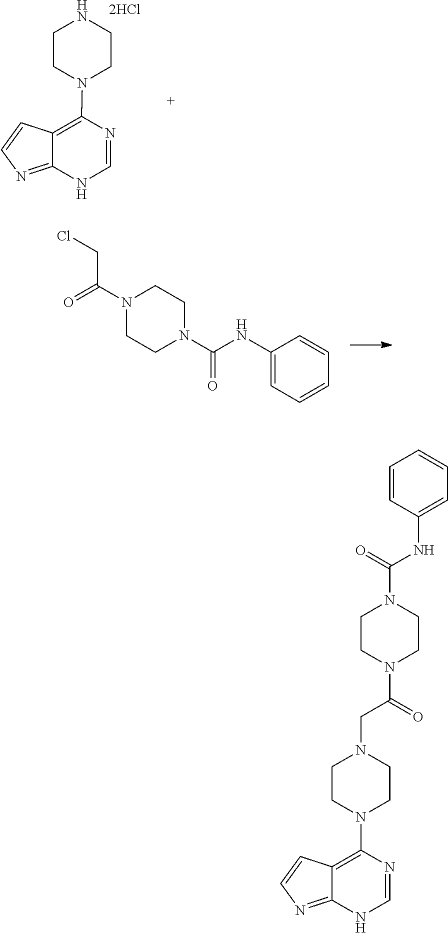 [SMILES: c1cc2c(N3CCNCC3)nc[nH]c-2n1 ; O=C(CCl)N1CCN(C(=O)Nc2ccccc2)CC1 ; O=C(CN1CCN(c2nc[nH]c3nccc2-3)CC1)N1CCN(C(=O)Nc2ccccc2)CC1]

To 1-[1H-pyrrolo[2,3-d]pyrimidin-4-yl]piperazine dihydrochloride (65 mg, 0.236 mmol) in dry DMSO (approximately 0.5 ml) was added 4-(2-chloroacetyl)-N-phenylpiperazine-1-carboxamide (71 mg, 0.253 mmol), diisopropylamine (133 mg, 1.315 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 40% to 90% of 0.1% NH₃/methanol in 0.1% aqueous NH₃. The title product was obtained in 54% yield (57.5 mg).

### Example 29. Synthesis of N-[3-(dimethylsulfamoyl)phenyl]-2-(4-{1H-pyrrolo[2,3-d]pyrimidin-4-yl}piperazin-1-yl)acetamide (Compound 29)

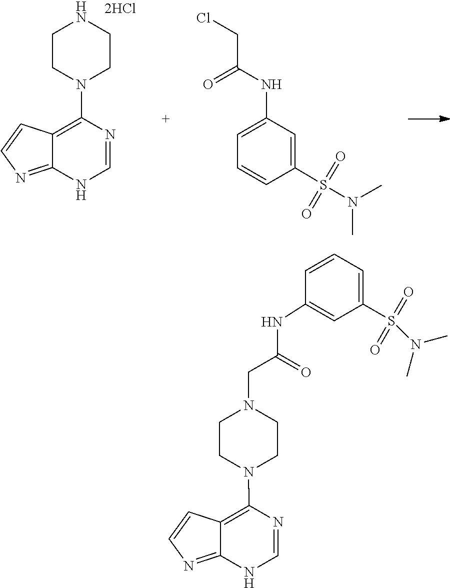

To 1-[1H-pyrrolo[2,3-d]pyrimidin-4-yl]piperazine dihydrochloride (66 mg, 0.24 mmol) in dry DMSO (approximately 0.5 ml) was added 2-chloro-N-[3-(dimethylsulfamoyl)phenyl]acetamide (69 mg, 0.25 mmol), diisopropylamine (121 mg, 1.197 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 30% to 80% of 0.1% NH3/methanol in 0.1% aqueous NH3. The title product was obtained in 41% yield (43.5 mg).

### Example 30. Synthesis of N-[3-(morpholine-4-sulfonyl)phenyl]-2-(4-{1H-pyrrolo[2,3-d]pyrimidin-4-yl}piperazin-1-yl)acetamide (Compound 30)

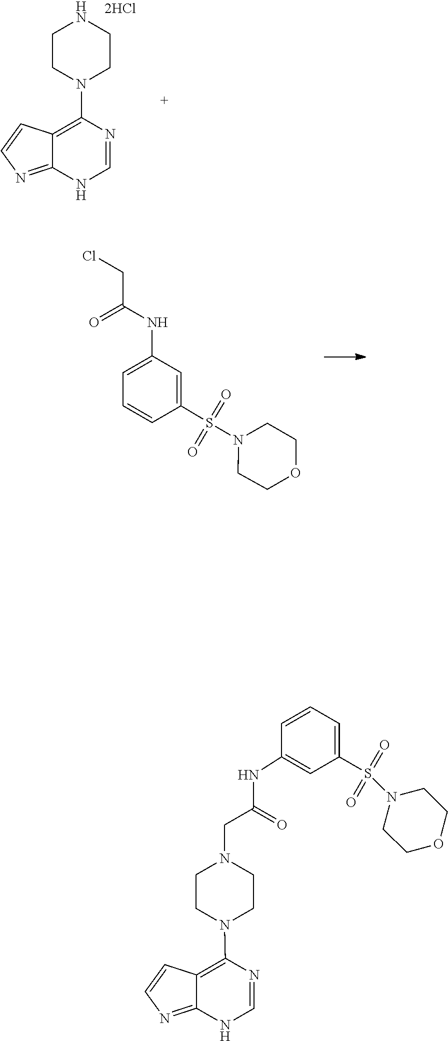 [SMILES: c1cc2c(N3CCNCC3)nc[nH]c-2n1 ; O=C(CCl)Nc1cccc(S(=O)(=O)N2CCOCC2)c1 ; O=C(CN1CCN(c2nc[nH]c3nccc2-3)CC1)Nc1cccc(S(=O)(=O)N2CCOCC2)c1]

To 1-[1H-pyrrolo[2,3-d]pyrimidin-4-yl]piperazine dihydrochloride (59 mg, 0.214 mmol) in dry DMSO (approximately 0.5 ml) was added 2-chloro-N-[3-(morpholine-4-sulfonyl)phenyl]acetamide (62 mg, 0.195 mmol), diisopropylamine (109 mg, 1.108 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 30% to 80% of 0.1% NH₃/methanol in 0.1% aqueous NH₃. The title product was obtained in 36% yield (37.5 mg).

### Example 31. Synthesis of N-[4-(piperidine-1-sulfonyl)phenyl]-2-(4-{1H-pyrrolo[2,3-d]pyrimidin-4-yl}piperazin-1-yl)acetamide (Compound 31)

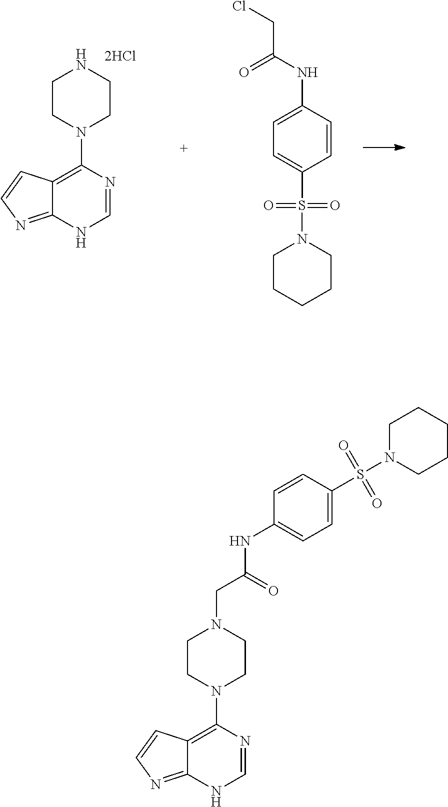 [SMILES: c1cc2c(N3CCNCC3)nc[nH]c-2n1 ; O=C(CCl)Nc1ccc(S(=O)(=O)N2CCCCC2)cc1 ; O=C(CN1CCN(c2nc[nH]c3nccc2-3)CC1)Nc1ccc(S(=O)(=O)N2CCCCC2)cc1]

To 1-[1H-pyrrolo[2,3-d]pyrimidin-4-yl]piperazine dihydrochloride (54 mg, 0.196 mmol) in dry DMSO (approximately 0.5 ml) was added 2-chloro-N-[4-(piperidine-1-sulfonyl)phenyl]acetamide (65 mg, 0.206 mmol), diisopropylamine (102 mg, 1.01 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 50% to 100% of 0.1% NH₃/methanol in 0.1% aqueous NH₃. The title product was obtained in 52% yield (49.8 mg).

### Example 32. Synthesis of N-[3-(pyrrolidine-1-sulfonyl)phenyl]-2-(4-{1H-pyrrolo[2,3-d]pyrimidin-4-yl}piperazin-1-yl)acetamide (Compound 32)

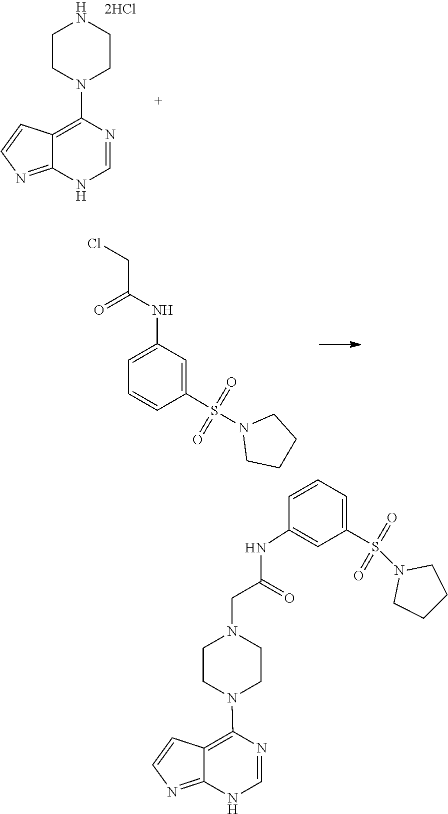

To 1-[1H-pyrrolo[2,3-d]pyrimidin-4-yl]piperazine dihydrochloride (68 mg, 0.247 mmol) in dry DMSO (approximately 0.5 ml) was added 2-chloro-N-[3-(pyrrolidine-1-sulfonyl)phenyl]acetamide (78 mg, 0.258 mmol), diisopropylamine (129 mg, 1.276 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 40% to 90% of 0.1% NH₃/methanol in 0.1% aqueous NH₃. The title product was obtained in 38% yield (44.4 mg).

### Example 33. Synthesis of N-[4-(2-phenyldiazen-1-yl)phenyl]-2-(4-{1H-pyrrolo[2,3-d]pyrimidin-4-yl}piperazin-1-yl)acetamide (Compound 33)

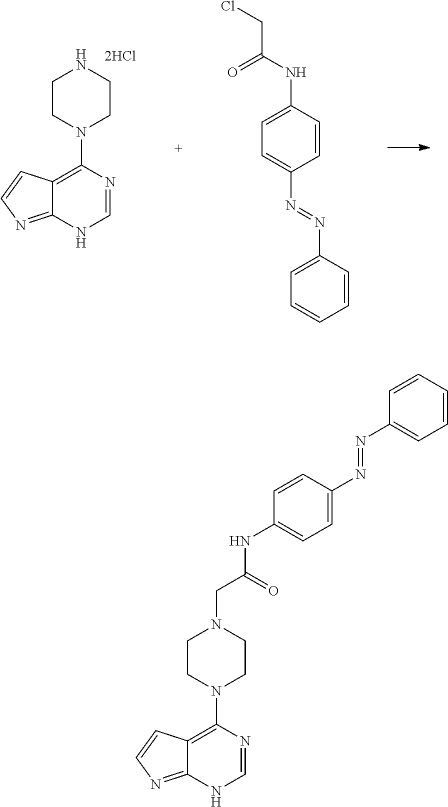 [SMILES: c1cc2c(N3CCNCC3)nc[nH]c-2n1 ; O=C(CCl)Nc1ccc(/N=N/c2ccccc2)cc1 ; O=C(CN1CCN(c2nc[nH]c3nccc2-3)CC1)Nc1ccc(/N=N/c2ccccc2)cc1]

To 1-[1H-pyrrolo[2,3-d]pyrimidin-4-yl]piperazine dihydrochloride (61 mg, 0.222 mmol) in dry DMSO (approximately 0.5 ml) was added 2-chloro-N-[4-(2-phenyldiazen-1-yl)phenyl]acetamide (67 mg, 0.245 mmol), diisopropylamine (112 mg, 1.108 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 60% to 100% of 0.1% NH₃/methanol in 0.1% aqueous NH₃. The title product was obtained in 68% yield (66 mg).

### Example 34. Synthesis of N-[4-chloro-3-(dimethylsulfamoyl)phenyl]-2-(4-{1H-pyrrolo[2,3-d]pyrimidin-4-yl}piperazin-1-yl)acetamide (Compound 34)

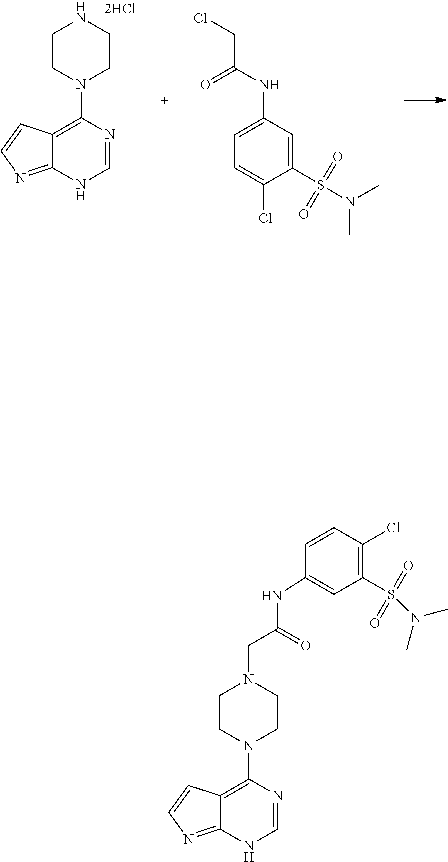 [SMILES: c1cc2c(N3CCNCC3)nc[nH]c-2n1 ; CN(C)S(=O)(=O)c1cc(NC(=O)CCl)ccc1Cl ; CN(C)S(=O)(=O)c1cc(NC(=O)CN2CCN(c3nc[nH]c4nccc3-4)CC2)ccc1Cl]

To 1-[1H-pyrrolo[2,3-d]pyrimidin-4-yl]piperazine dihydrochloride (58 mg, 0.211 mmol) in dry DMSO (approximately 0.5 ml) was added 2-chloro-N-[4-chloro-3-(dimethylsulfamoyl)phenyl]acetamide (65 mg, 0.21 mmol), diisopropylamine (109 mg, 1.078 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 40% to 90% of 0.1% NH₃/methanol in 0.1% aqueous NH₃. The title product was obtained in 25% yield (25.1 mg).

### Example 35. Synthesis of N-[3-(dimethylsulfamoyl)-4-methylphenyl]-2-(4-{1H-pyrrolo[2,3-d]pyrimidin-4-yl}piperazin-1-yl)acetamide (Compound 35)

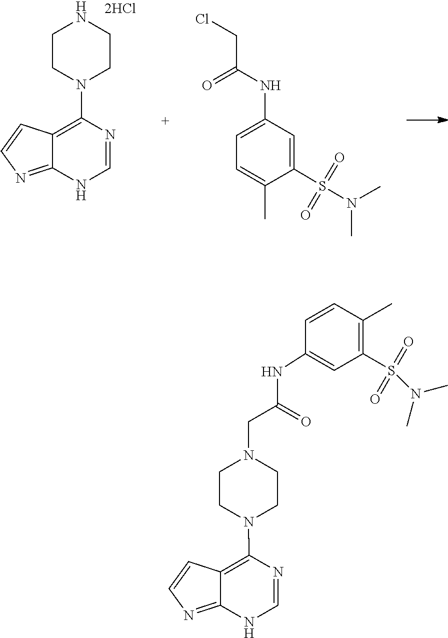 [SMILES: c1cc2c(N3CCNCC3)nc[nH]c-2n1 ; Cc1ccc(NC(=O)CCl)cc1S(=O)(=O)N(C)C ; Cc1ccc(NC(=O)CN2CCN(c3nc[nH]c4nccc3-4)CC2)cc1S(=O)(=O)N(C)C]

To 1-[1H-pyrrolo[2,3-d]pyrimidin-4-yl]piperazine dihydrochloride (60 mg, 0.218 mmol) in dry DMSO (approximately 0.5 ml) was added 2-chloro-N-[3-(dimethylsulfamoyl)-4-methylphenyl]acetamide (68 mg, 0.234 mmol), diisopropylamine (117 mg, 1.157 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 40% to 90% of 0.1% NH₃/methanol in 0.1% aqueous NH₃. The title product was obtained in 54% yield (53.8 mg).

### Example 36. Synthesis of N-[3-(dimethylsulfamoyl)phenyl]-2-(4-{1H-pyrrolo[2,3-d]pyrimidin-4-yl}piperazin-1-yl)propenamide (Compound 36)

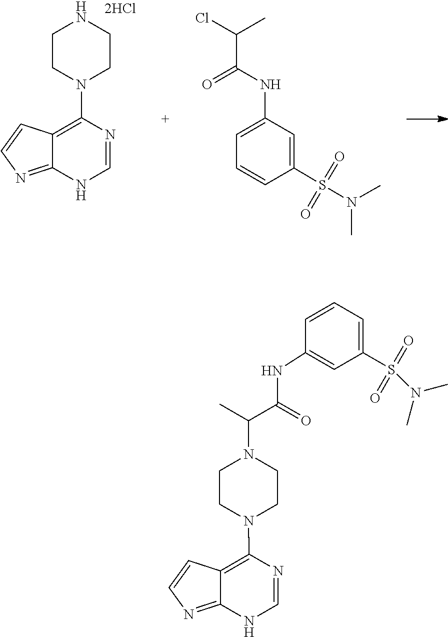 [SMILES: c1cc2c(N3CCNCC3)nc[nH]c-2n1 ; CC(Cl)C(=O)Nc1cccc(S(=O)(=O)N(C)C)c1 ; CC(C(=O)Nc1cccc(S(=O)(=O)N(C)C)c1)N1CCN(c2nc[nH]c3nccc2-3)CC1]

To 1-[1H-pyrrolo[2,3-d]pyrimidin-4-yl]piperazine dihydrochloride (66 mg, 0.24 mmol) in dry DMSO (approximately 0.5 ml) was added 2-chloro-N-[3-(dimethylsulfamoyl)phenyl]propanamide (74 mg, 0.255 mmol), diisopropylamine (128 mg, 1.266 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 40% to 90% of 0.1% NH₃/methanol in 0.1% aqueous NH₃. The title product was obtained in 53% yield (57.7 mg).

### Example 37. Synthesis of N-{4-[methyl(propan-2-yl)sulfamoyl]phenyl}-2-(4-{1H-pyrrolo[2,3-d]pyrimidin-4-yl}piperazin-1-yl)acetamide (Compound 37)

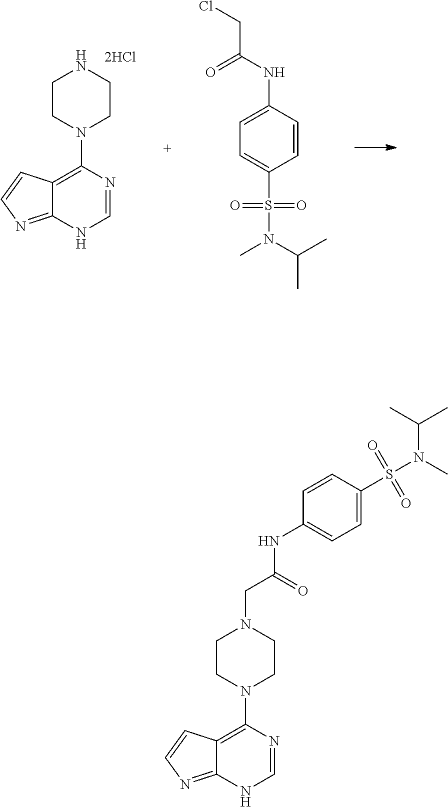 [SMILES: c1cc2c(N3CCNCC3)nc[nH]c-2n1 ; CC(C)N(C)S(=O)(=O)c1ccc(NC(=O)CCl)cc1 ; CC(C)N(C)S(=O)(=O)c1ccc(NC(=O)CN2CCN(c3nc[nH]c4nccc3-4)CC2)cc1]

To 1-[1H-pyrrolo[2,3-d]pyrimidin-4-yl]piperazine dihydrochloride (54 mg, 0.196 mmol) in dry DMSO (approximately 0.5 ml) was added 2-chloro-N-{4-[methyl(propan-2-yl)sulfamoyl]phenyl}acetamide (64 mg, 0.21 mmol), diisopropylamine (105 mg, 1.04 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 50% to 100% of 0.1% NH₃/methanol in 0.1% aqueous NH₃. The title product was obtained in 53% yield (49.2 mg).

### Example 38. Synthesis of 3-(4-{1H-pyrrolo[2,3-d]pyrimidin-4-yl}piperazin-1-yl)-N-(3-sulfamoylphenyl)propenamide (Compound 38)

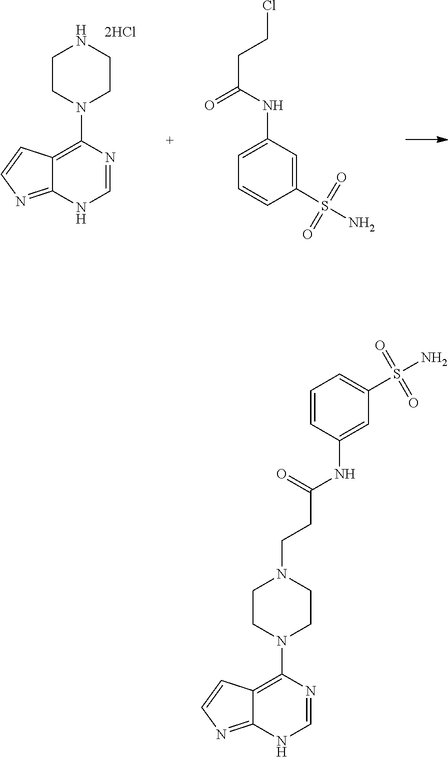 [SMILES: c1cc2c(N3CCNCC3)nc[nH]c-2n1 ; NS(=O)(=O)c1cccc(NC(=O)CCCl)c1 ; NS(=O)(=O)c1cccc(NC(=O)CCN2CCN(c3nc[nH]c4nccc3-4)CC2)c1]

To 1-[1H-pyrrolo[2,3-d]pyrimidin-4-yl]piperazine dihydrochloride (64 mg, 0.233 mmol) in dry DMSO (approximately 0.5 ml) was added 3-chloro-N-(3-sulfamoylphenyl)propanamide (70 mg, 0.267 mmol), diisopropylamine (126 mg, 1.246 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 30% to 80% of 0.1% NH₃/methanol in 0.1% aqueous NH₃. The title product was obtained in 55% yield (54.7 mg).

### Example 39. Synthesis of N-[3-(pyrrolidine-1-sulfonyl)phenyl]-3-(4-{1H-pyrrolo[2,3-d]pyrimidin-4-yl}piperazin-1-yl)propenamide (Compound 39)

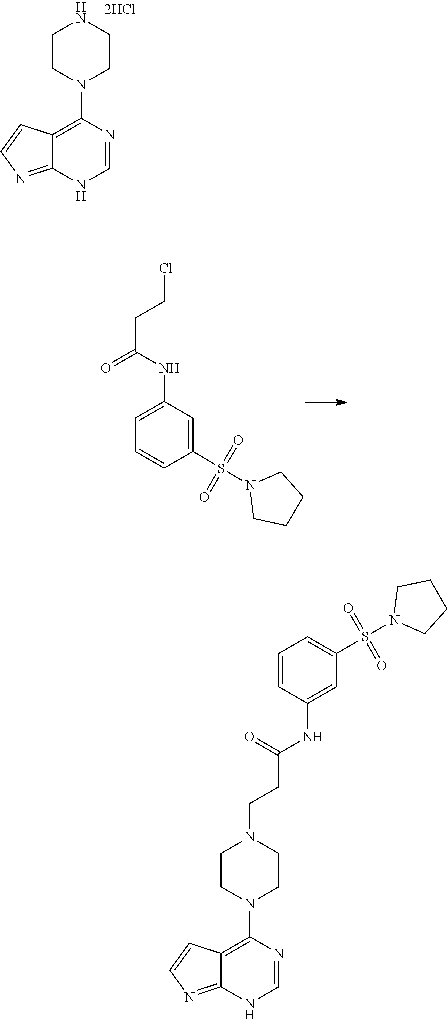 [SMILES: c1cc2c(N3CCNCC3)nc[nH]c-2n1 ; O=C(CCCl)Nc1cccc(S(=O)(=O)N2CCCC2)c1 ; O=C(CCN1CCN(c2nc[nH]c3nccc2-3)CC1)Nc1cccc(S(=O)(=O)N2CCCC2)c1]

To 1-[1H-pyrrolo[2,3-d]pyrimidin-4-yl]piperazine dihydrochloride (55 mg, 0.20 mmol) in dry DMSO (approximately 0.5 ml) was added 3-chloro-N-(3-sulfamoylphenyl)propanamide (64 mg, 0.202 mmol), diisopropylamine (104 mg, 1.028 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 50% to 100% of 0.1% NH3/methanol in 0.10% aqueous NH₃. The title product was obtained in 57% yield (54.9 mg).

### Example 40. Synthesis of N-[4-(dimethylsulfamoyl)phenyl]-3-(4-{1H-pyrrolo[2,3-d]pyrimidin-4-yl}piperazin-1-yl)propenamide (Compound 40)

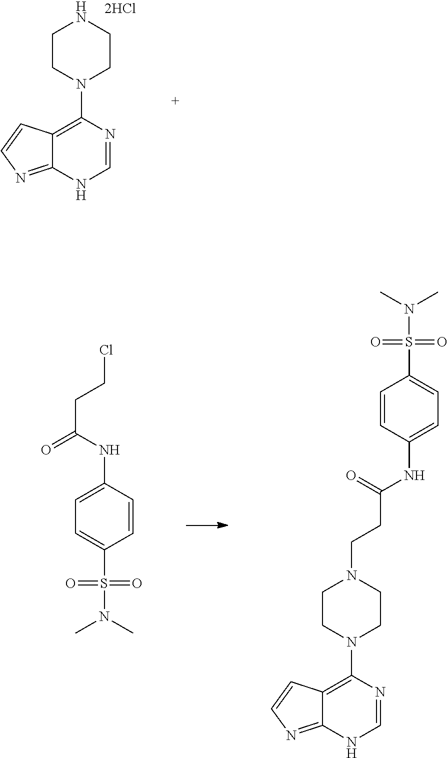 [SMILES: c1cc2c(N3CCNCC3)nc[nH]c-2n1 ; CN(C)S(=O)(=O)c1ccc(NC(=O)CCCl)cc1 ; CN(C)S(=O)(=O)c1ccc(NC(=O)CCN2CCN(c3nc[nH]c4nccc3-4)CC2)cc1]

To 1-[1H-pyrrolo[2,3-d]pyrimidin-4-yl]piperazine dihydrochloride (62 mg, 0.225 mmol) in dry DMSO (approximately 0.5 ml) was added 3-chloro-N-[4-(dimethylsulfamoyl)phenyl]propanamide (73 mg, 0.252 mmol), diisopropylamine (122 mg, 1.206 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 40% to 90% of 0.1% NH₃/methanol in 0.1% aqueous NH₃. The title product was obtained in 53% yield (54.3 mg).

### Example 41. Synthesis of N-{[2-(dimethylsulfamoyl)phenyl]methyl}-2-(4-{1H-pyrrolo[2,3-d]pyrimidin-4-yl}piperazin-1-yl)acetamide (Compound 41)

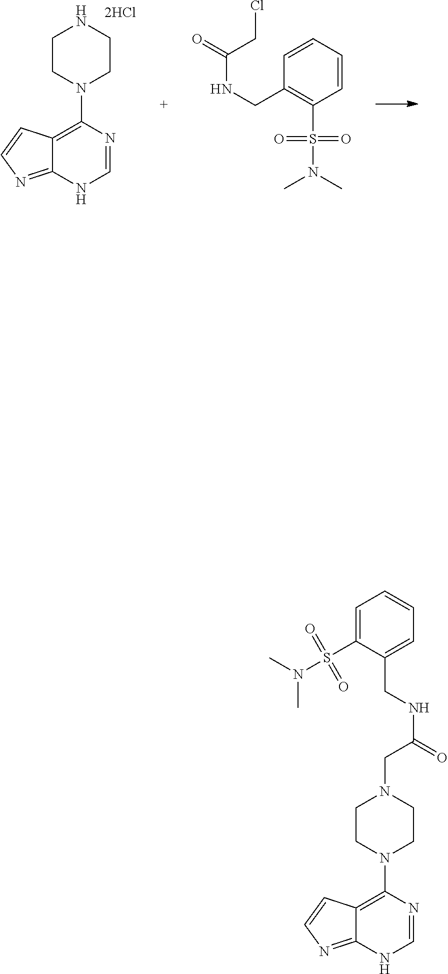 [SMILES: c1cc2c(N3CCNCC3)nc[nH]c-2n1 ; CN(C)S(=O)(=O)c1ccccc1CNC(=O)CCl ; CN(C)S(=O)(=O)c1ccccc1CNC(=O)CN1CCN(c2nc[nH]c3nccc2-3)CC1]

To 1-[1H-pyrrolo[2,3-d]pyrimidin-4-yl]piperazine dihydrochloride (66 mg, 0.24 mmol) in dry DMSO (approximately 0.5 ml) was added 2-chloro-N-{[2-(dimethylsulfamoyl)phenyl]methyl}acetamide (76 mg, 0.262 mmol), diisopropylamine (129 mg, 1.276 mmol), and sodium iodide (0.2 eq.). The reaction mixture was sealed and heated for 16 hours at 100° C. The reaction mixture was then cooled, filtered, and purified by HPLC on an XBridge BEH C18 5 mm 130Å column (Waters) using a gradient from 30% to 80% of 0.1% NH₃/methanol in 0.1% aqueous NH₃. The title product was obtained in 49% yield (54.3 mg).

### Example 42. JAK Enzymatic Assays

All enzyme assays were conducted according to Eurofins Discovery's KinaseProfiler™ and IC₅₀Profiler™ services. Test compound stocks (10 mM in DMSO) were incubated with the reaction conditions described below at 9 different concentrations (half-log dilution series) to enable the generation and fitting of IC₅₀ curves.

Human JAK-1 (Uniprot ID: P23458) was incubated with 20 mM Tris/HCl pH 7.5, 0.2 mM EDTA, 500 uM GEEPLYWSFPAKKK (SEQ ID NO.: 1) peptide substrate, 10 mM magnesium acetate and [gamma-33P]-ATP (specific activity and concentration as required). The reaction was initiated by the addition of the Mg/ATP mix. After incubation for 40 minutes at room temperature, the reaction was stopped by the addition of phosphoric acid to a concentration of 0.5%. An aliquot of the reaction was then spotted onto a filter and washed four times for 4 minutes in 0.425% phosphoric acid and once with methanol prior to drying and scintillation counting. A known inhibitor, Staurosporine, was used as a positive control for the assay.

Human JAK-2 (Uniprot ID: 060674) was incubated with 8 mM MOPS pH 7.0, 0.2 mM EDTA, 100 uM KTFCGTPEYLAPEVRREPRILSEEEQEMFRDFDYIADWC (SEQ ID NO: 2) peptide substrate, 10 mM magnesium acetate and [gamma-33P]-ATP (specific activity and concentration as required). The reaction was initiated by the addition of the Mg/ATP mix. After incubation for 40 minutes at room temperature, the reaction was stopped by the addition of phosphoric acid to a concentration of 0.5%. An aliquot of the reaction was then spotted onto a filter and washed four times for 4 minutes in 0.425% phosphoric acid and once with methanol prior to drying and scintillation counting.

Human JAK-3 (Uniprot ID: P52333) was incubated with 8 mM MOPS pH 7.0, 0.2 mM EDTA, 500 uM GGEEEEYFELVKKKK (SEQ ID NO: 3) peptide substrate, 10 mM magnesium acetate and [gamma-33P]-ATP (specific activity and concentration as required). The reaction was initiated by the addition of the Mg/ATP mix. After incubation for 40 minutes at room temperature, the reaction was stopped by the addition of phosphoric acid to a concentration of 0.5%. An aliquot of the reaction was then spotted onto a filter and washed four times for 4 minutes in 0.425% phosphoric acid and once with methanol prior to drying and scintillation counting.

Human TYK-2 (Uniprot ID: P29597) was incubated with 8 mM MOPS pH 7.0, 0.2 mM EDTA, 250 μM GGMEDIYFEFMGGKKK (SEQ ID NO: 4) peptide substrate, 10 mM magnesium acetate and [γ-33P]-ATP (specific activity and concentration as required). The reaction was initiated by the addition of the Mg/ATP mix. After incubation for 40 minutes at room temperature, the reaction was stopped by the addition of phosphoric acid to a concentration of 0.5%. An aliquot of the reaction was then spotted onto a filter and washed four times for 4 minutes in 0.425% phosphoric acid and once with methanol prior to drying and scintillation counting.

Data was then imported into an internal application where each data point was expressed as % inhibition based on uninhibited and no enzyme controls. Using this protocol, IC₅₀ results were generated for example compounds of the disclosure. Inhibition values for the JAK enzymes (IC₅₀ values) for each compound were stratified into four groups. The group numbers are provided in Table 1, along with the selectivity IC₅₀ ratios for JAK1 versus JAK2.

TABLE 1
JAK inhibitory activity and selectivity

| Compound | | | | | JAK1/JAK2 |
|---|---|---|---|---|---|
| # | JAK1 | JAK2 | JAK3 | TYK2 | selectivity |
| 1 | 3 | 4 | 4 | 4 | >20 |
| 2 | 1 | 4 | 4 | 4 | 25 |
| 3 | 2 | 4 | 4 | 4 | 26 |
| 4 | 2 | 4 | 4 | 4 | 24 |
| 5 | 2 | 4 | 4 | 4 | >38 |
| 6 | 1 | 4 | 4 | 4 | 62 |
| 7 | 1 | 3 | 4 | 4 | 13 |
| 8 | 1 | 3 | 3 | 4 | 7 |
| 9 | 1 | 3 | 3 | 3 | 12 |
| 10 | 2 | 4 | 4 | 4 | 15 |
| 11 | 1 | 4 | 3 | 4 | 9 |
| 12 | 1 | 3 | 3 | 4 | 4 |
| 13 | 1 | 4 | 4 | 4 | 13 |
| 14 | 2 | 4 | 4 | 4 | 17 |
| 15 | 2 | 4 | N/A | N/A | 9 |
| 16 | 2 | 4 | N/A | N/A | 19 |
| 17 | 2 | 4 | 4 | 4 | 7 |
| 18 | 2 | 1 | 3 | 4 | 4 |
| 19 | 1 | 4 | 4 | N/A | 8 |
| 20 | 3 | 4 | 4 | 3 | 2 |
| 23 | 4 | 4 | N/A | N/A | — |
| 24 | 4 | 4 | N/A | N/A | — |
| 25 | 1 | 1 | N/A | N/A | 1 |
| 26 | 2 | 4 | N/A | N/A | >22 |
| 27 | 1 | 4 | N/A | N/A | 14 |
| 28 | 2 | 4 | N/A | N/A | 19 |
| 29 | 3 | 4 | N/A | N/A | 11 |
| 30 | 4 | 4 | N/A | N/A | 6 |
| 31 | 1 | 4 | N/A | N/A | 4 |
| 32 | 2 | 4 | N/A | N/A | 23 |
| 33 | 1 | 4 | N/A | N/A | >100 |
| 34 | 2 | 4 | N/A | N/A | 23 |
| 35 | 3 | 4 | N/A | N/A | 11 |
| 36 | 4 | 4 | N/A | N/A | 9 |
| 37 | 2 | 4 | N/A | N/A | 15 |
| 38 | 3 | 4 | N/A | N/A | 6 |
| 39 | 3 | 4 | N/A | N/A | 1 |
| 40 | 2 | 4 | N/A | N/A | 11 |
| 41 | 3 | 4 | N/A | N/A | 10 |

Affinity (IC₅₀) categories: 1: <250 nM; 2: 250 to 500 nM: 3: 500 to 1000 nM; 4: >1000 nM

With reference to Table 1, good affinity was observed for many of the compounds (IC₅₀<250 nM) along with many instances of greater than 5 or greater than 10-fold selectivity for JAK1 versus JAK2. As discussed herein above, the very high homology for human versus canine subtypes (approaching 100% at active site) is believed to translate directly to the corresponding canine activity, with the human assay serving as a convenient and readily available surrogate.

### Example 43. Canine: In a Vitro T-Cell Proliferation Assay

T-cell activation plays a key role in a variety of inflammatory and autoimmune disorders as well as asthma, allergies and pruritus Since T-cell activation can, in part, can be triggered by cytokines that signal through the JAK-STAT pathway, a JAK inhibitor could be effective against such diseases involving aberrant T-cell activation.

### Methods

Canine whole blood is collected in sodium heparin tubes from 29 beagle dogs and 23 mixed breed dogs. Whole blood (20 μL) is plated in 96-well plates (Costar 3598) with 180 μL of medium (RPMI 1640, Gibco #21870-076, with 1% heat inactivated fetal bovine serum, Gibco #10082-39, 292 μg/ml L-glutamine, Gibco #250030-081, 100 u/ml penicillin and 100 μg streptomycin per ml, Gibco #15140-122) containing vehicle control or test compound (0.001 to 10 M), concanavalin A (ConA; 1 μg/ml, Sigma C75275), and canine interleukin-2 (IL-2; 50 ng/ml, R&D Systems 1815-CL/CF). Wells containing whole blood, medium with vehicle control and no ConA or IL-2 are used as background controls. Plates are incubated at 37 C for 48 hours. Tritiated thymidine. 0.4 μCi/well (Perkin Elmer. NetO27A-005MC), is added for 20 additional hours. Plates are frozen and then thawed, washed and filtered using a Brandel MLR-96 cell harvester and prewet filter mats (Wallac 1205-401, Perkin Elmer). Filters are dried at 60° C. for one hour (Precision 16EG convection oven) and placed into filter sample bags (Wallac 1205-411, Perkin Elmer) with 10 mL of scintillant (Wallac 1205-440, Perkin Elmer). Sealed filters are counted on a LKB Wallac 1205 Betaplate liquid scintillation counter. Data are collected via Gterm Betaplate progranm v1.1 (Wallac copyright 1989-1990) and transformed into percent inhibition. Data are graphically displayed as percent inhibition using GraphPad Prism 4.0, and IC₅₀ curves fitted using a point-to-point analysis.

### Example 44. Cell Proliferation Inhibitory Assay

### Feline Cell Lines

The MYA-I and FETJ are feline T-lymphoblast cell lines obtained from ATCC (Manassas, VA). These cells are cultured in RPMI 1640 complete media supplemented with 10% FBS at 37° C. in a humidified incubator with 5% CO).

### Ex Vivo Canine Lymphoma Nodal Tissue

Malignant lymph nodes are excised by a veterinarian, placed into transport media (Advanced RPMI 1640 complete medium supplemented with 10% Fetal Bovine Serum (FBS), 100U/mL penicillin, 100 ug/mL streptomycin and 0.25 ug/mL Amphotercin B (Invitrogen/Gibco®). Nodes are processed within 24 hours of removal by mincing into tiny pieces and passing through a tissue sieve. Cell suspensions are spun at 200×g, supernatant was removed, and the cell pellet is resuspended in NH₄Cl solution for 10 minutes at room temperature. The cell suspension is pelleted by centrifugation; the NH₄Cl was removed and the pellet washed once with Hanks Balanced Salt Solution (HBSS), followed by re-suspension in Proliferation Medium (Advanced RPMI complete, 1% FBS, 50 nM 2-Mercaptoethanol, 100 U/mL penicillin, 100 ug/mL streptomycin and 0.25 ug/mL Amphotercin B). The cell suspension is then passed through a 100 μm nylon cell strainer (BD-Falcon) and counted using a hemacytometer. Cells are cultured in either Proliferation Medium alone, Proliferation Medium supplemented with 0.005% Pansorbin® (Heat inactivated, formalin-fixed Staphylococcus Aureus cells (SAC), Calbiochem), and 10 ng/mL canine IL-2 (R&D Systems), or Proliferation Medium supplemented with 125 ng/mL concavalin A (Sigma) and 125 ng/mL lipopolysacharride (LPS; Calbiochern).

Cells cultured in medium described above are plated in 96-well Costar plates (Corning) at a density of 1×10³ cells/well (feline cell lines) or 2×10⁵ cells/well (lymph node cells) and exposed to various concentrations of test compounds for up to 5 days at 37° C. in a humidified incubator with 5% CO₂. Effects on proliferation are determined using the CellTiter 96® Aqueous Non-Radioactive Cell Proliferation Assay (Promega) according to manufacturer's instructions. In general, proliferation is indirectly measured using a soluble tetrazolium salt (MTS) and an electron coupling agent. MTS bioreduction into a formazan product soluble in tissue culture medium is monitored by absorbance at 490 nM on a Spectramax plate reader using Softmax Pro 4.6 software (Molecular Devices). Data are graphically displayed as percent DMSO control using GraphPad Prism 4.00, and IC₅₀ curves are fitted using a non-linear regression model with a sigmoidal dose response.

### Example 45. Reducing Flea-Associate Pruritus and Dermatitis Assay

Flea-associated pruritus and dermatitis are common skin conditions in dogs. Pruritus is one of the most severe clinical signs associated with flea associated dermatitis, and continued scratching, face rubbing, and foot chewing can lead to a variety of changes to the skin such as erythema, edema, alopecia, lichenification, and hyper pigmentation. Flea-associated pruritus and dermatitis can be induced experimentally. In these models, inflammatory cells and cytokines have been shown to mediate immune reactions to allergens. Therefore, a JAK inhibitor that inhibits signal transduction of pruritogenic and proinflammatory cytokine receptors could be effective in inhibiting, reducing or minimizing flea-associated pruritus and dermatitis.

### Study Design

Twenty-eight male and female dogs of mixed breeds ranging in weight from 5-35 kg and greater than one year of age are infested with approximately 100 unfed adult cat fleas (Ctenocephalides felis) 14 days prior to the start of dosing and reinfested with 30 fleas per dog every 4 days throughout the study. Seven days before dosing, twenty-four dogs are randomized into three different treatment groups, placebo, 0.5 mg/kg or 0.25 mg/kg of the test compound, based on visual analog scale (VAS) scores for skin lesions. Treatments are given orally twice a day for 28 days, and pruritic behavior as well as erythema and skin lesions is assessed during the study. Pruritic behavior is recorded by placing dogs into pens with video recording capability and recording their activity over 4 hours. Pruritic activity is quantitated by determining how many seconds the dogs spent scratching. Skin lesions are recorded by image capture of abdominal, inguinal region and the severity ranked according to a visual analog scale (VAS).

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A compound having a structure according to Formula I: 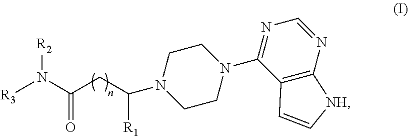
or a pharmaceutically acceptable salt or solvate thereof, wherein:
R₁ is H or optionally substituted C₁-C₃ alkyl;
n=0 or 1;
R₂ is H or CH₃;
R₃ is C₁-C₄ alkyl, phenyl, 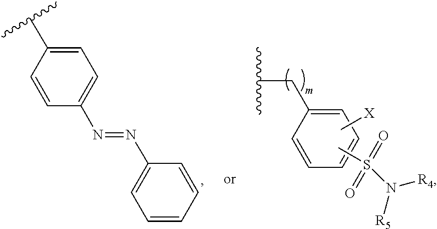
wherein:
m is 0 or 1;
X is H, F, Cl, OCH₃, or CH₃,
R₄ is H or CH₃,
R₅ is H, C₁-C₄ alkyl, C₃-C₅ cycloalkyl, CH₂CH₂OCH₃, or 2-pyrimidinyl; or
R₄ and R₅, together with the included N atom, form a 4-, 5- or 6-membered ring,
wherein said 6-membered ring optionally includes one additional heteroatom selected from N and O; or
R₂ and R₃, together with the included N atom, form a 6-membered ring, optionally substituted with C₁-C₄ alkyl, C₇ to C₁₂ aralkyl, or —C(O)—NR₆R₇,
wherein:
R₆ is H or C₁-C₄ alkyl;
R₇ is H, C₁-C₄ alkyl, phenyl; or
R₆ and R₇, together with the included N atom, form a 6-membered ring which may optionally be substituted with C₁-C₄alkyl, and wherein said 6-membered ring optionally includes one additional N atom.

2. The compound of claim 1, wherein:
R₂ is H; and
R₃ is:

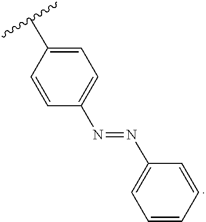 [SMILES: *c1ccc(/N=N/c2ccccc2)cc1]

3. The compound of claim 2, wherein n is 1 and R₁ is H.

4. The compound of claim 1, wherein R₃ is:

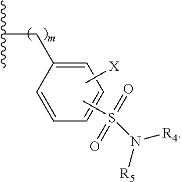

5. The compound of claim 4, wherein m is 0.

6. The compound of claim 5, wherein R₄ and R₅ are each H.

7. The compound of claim 6, selected from the group consisting of

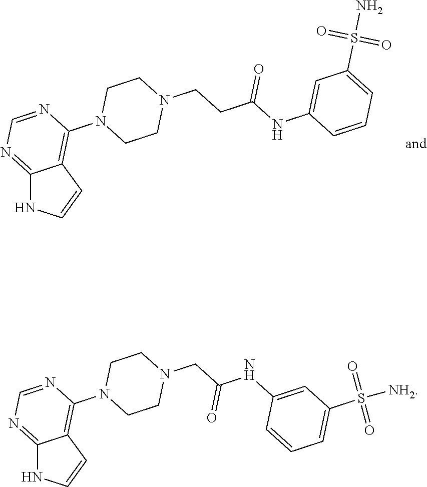 [SMILES: NS(=O)(=O)c1cccc(NC(=O)CCN2CCN(c3ncnc4[nH]ccc34)CC2)c1 ; NS(=O)(=O)c1cccc(NC(=O)CN2CCN(c3ncnc4[nH]ccc34)CC2)c1]

8. The compound of claim 4, wherein m is 1.

9. The compound of claim 8, wherein R₄ and R₅ are each CH₃.

10. The compound of claim 9, which has a structure

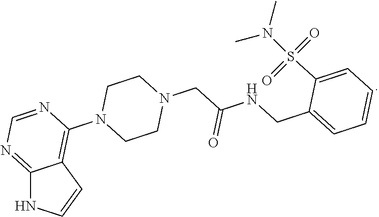 [SMILES: CN(C)S(=O)(=O)c1ccccc1CNC(=O)CN1CCN(c2ncnc3[nH]ccc23)CC1]

11. The compound of claim 4, wherein:
R₁ is H;
R₂ is H;
R₄ is H; and
R₅ is selected from the group consisting of C₁-C₄ alkyl, C₃-C₅ cycloalkyl, CH₂CH₂OCH₃, and 2-pyrimidinyl.

12. The compound of claim 4, wherein:
R₁ is CH₃;
R₂ is H;
R₄ is H; and
R₅ is selected from the group consisting of C₁-C₄ alkyl, C₃-C₅ cycloalkyl, CH₂CH₂OCH₃, and 2-pyrimidinyl.

13. The compound of claim 11, selected from the group consisting of

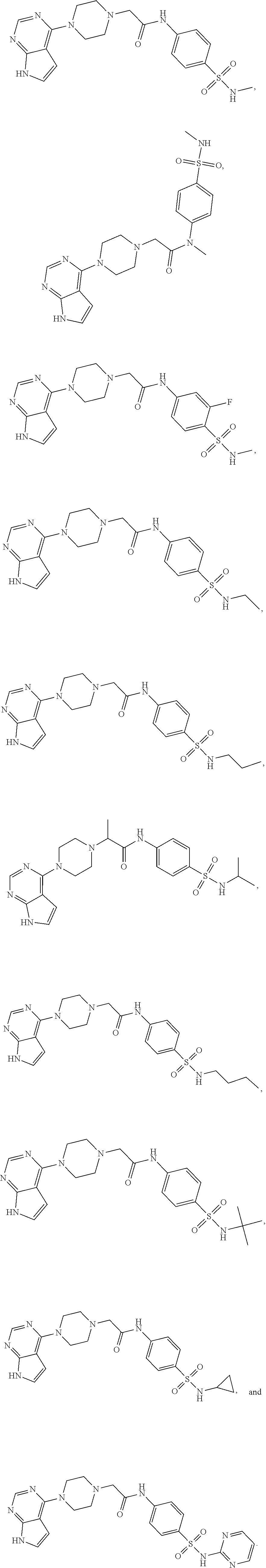

14. The compound of claim 4, wherein:
R₁ is H or CH₃;
R₂ is H; and
R₄ and R₅ are each independently selected from the group consisting of C₁-C₄ alkyl, C₃-C₅ cycloalkyl, CH₂CH₂OCH₃, and 2-pyrimidinyl.

15. The compound of claim 14, selected from the group consisting of

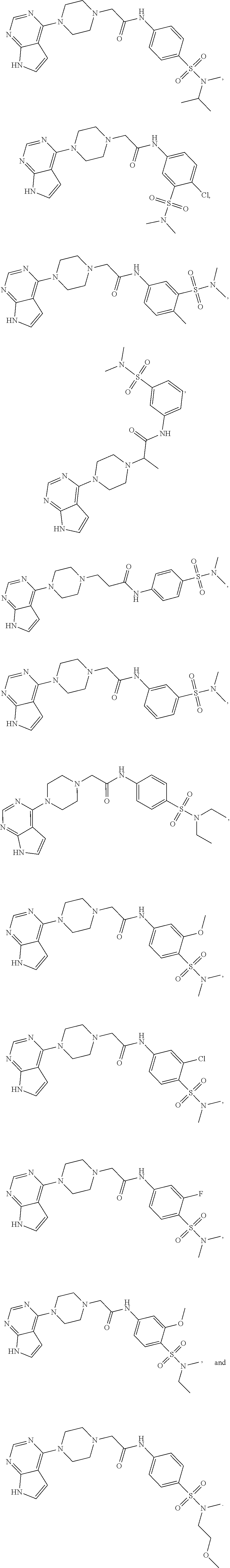

16. The compound of claim 4, wherein:
R₁ is H or CH₃;
R₂ is H; and
R₄ and R₅ together with the included N atom, form a 4-, 5- or 6-membered ring, wherein said 6-membered ring optionally includes one additional heteroatom selected from N and O.

17. The compound of claim 16, selected from the group consisting of

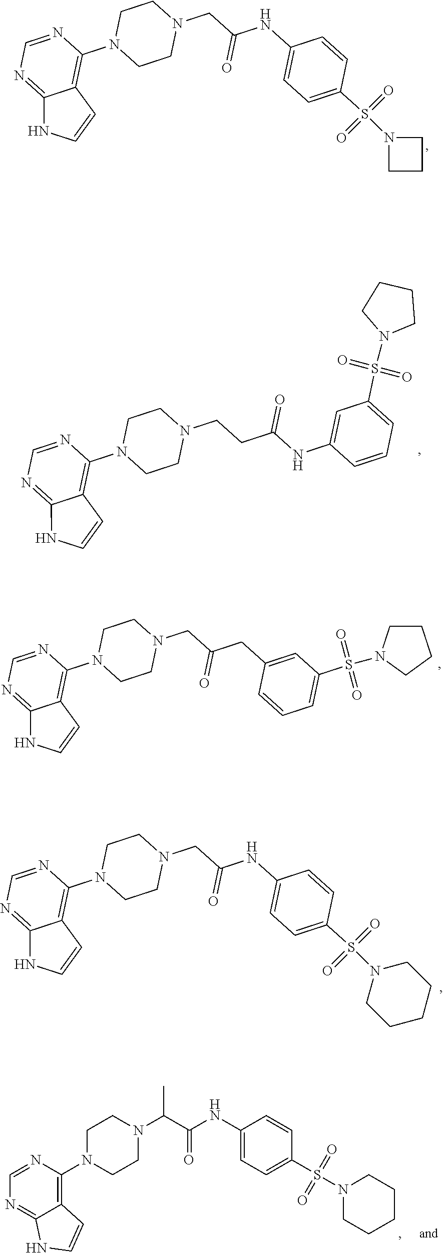 [SMILES: O=C(CN1CCN(c2ncnc3[nH]ccc23)CC1)Nc1ccc(S(=O)(=O)N2CCC2)cc1 ; O=C(CCN1CCN(c2ncnc3[nH]ccc23)CC1)Nc1cccc(S(=O)(=O)N2CCCC2)c1 ; O=C(Cc1cccc(S(=O)(=O)N2CCCC2)c1)CN1CCN(c2ncnc3[nH]ccc23)CC1 ; O=C(CN1CCN(c2ncnc3[nH]ccc23)CC1)Nc1ccc(S(=O)(=O)N2CCCCC2)cc1 ; CC(C(=O)Nc1ccc(S(=O)(=O)N2CCCCC2)cc1)N1CCN(c2ncnc3[nH]ccc23)CC1]

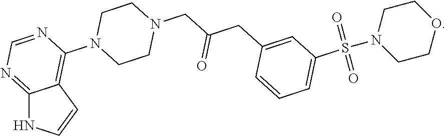 [SMILES: O=C(Cc1cccc(S(=O)(=O)N2CCOCC2)c1)CN1CCN(c2ncnc3[nH]ccc23)CC1]

18. The compound of claim 1, which is

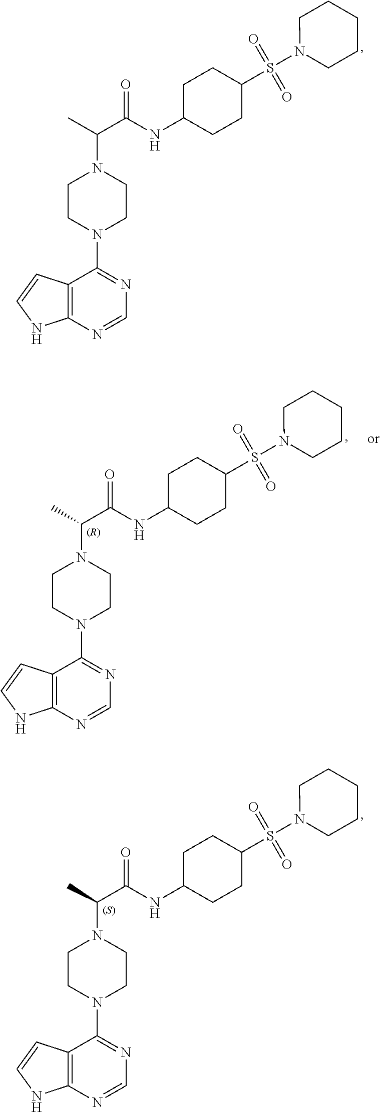

19. The compound of claim 1, which is

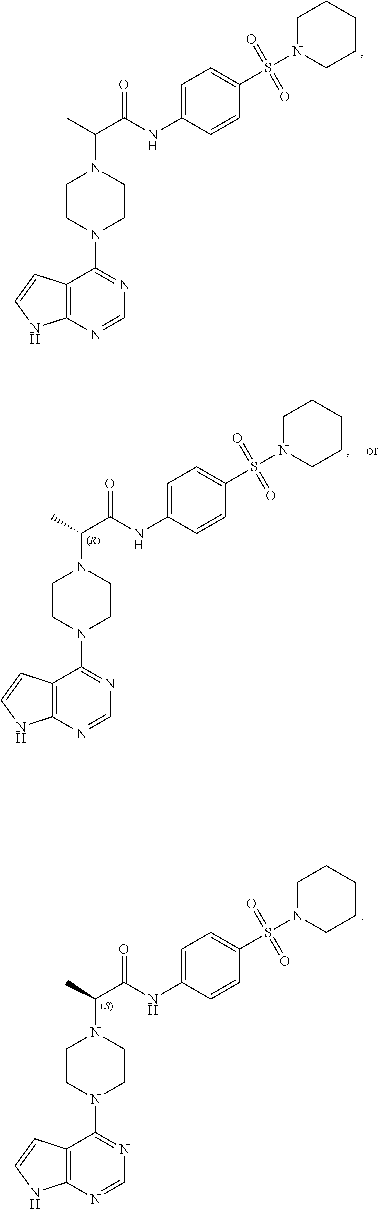

20. The compound of claim 1, wherein R₂ and R₃, together with the included N atom, form a 6-membered ring, optionally substituted with C₁-C₄ alkyl, C₇ to C₁₂ aralkyl, or —C(O)—NR₆R₇.

21. The compound of claim 20, having a structure according to Formula II: 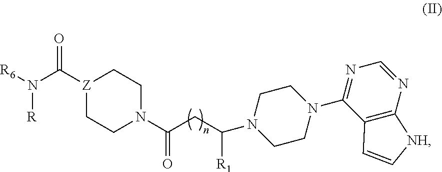
wherein Z is C or N.

22. The compound of claim 21, having a structure selected from the group consisting of

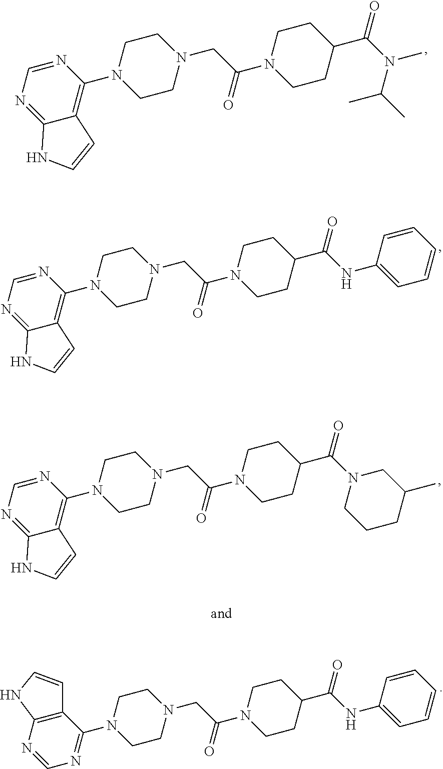 [SMILES: CC(C)N(C)C(=O)C1CCN(C(=O)CN2CCN(c3ncnc4[nH]ccc34)CC2)CC1 ; O=C(Nc1ccccc1)C1CCN(C(=O)CN2CCN(c3ncnc4[nH]ccc34)CC2)CC1 ; CC1CCCN(C(=O)C2CCN(C(=O)CN3CCN(c4ncnc5[nH]ccc45)CC3)CC2)C1 ; O=C(Nc1ccccc1)C1CCN(C(=O)CN2CCN(c3ncnc4[nH]ccc34)CC2)CC1]

23. The compound of claim 20, having a structure

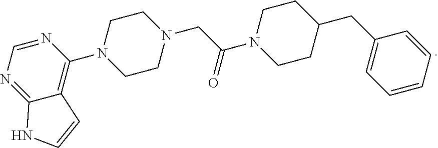 [SMILES: O=C(CN1CCN(c2ncnc3[nH]ccc23)CC1)N1CCC(Cc2ccccc2)CC1]

24. A method for treating allergic reactions, allergic dermatitis, atopic dermatitis, eczema, or pruritus in a mammal comprising administering to a mammal in need thereof a therapeutically effective amount of a compound of claim 1.
